(19) United States
(12) Patent Application Publication
(10) Pub. No.: US 2024/0349961 A1
(43) Pub. Date: Oct. 24, 2024

(54) WATERPROOF WASTE BAG FOR AUTONOMOUS VACUUM SYSTEMS

(71) Applicant: Matic Robots, Inc., Mountain View, CA (US)

(72) Inventors: Karthik Chandrashekaraiah, San Francisco, CA (US); Anshuman Kumar, San Francisco, CA (US); Yu Yang Liu, Sunnyvale, CA (US); Shu Yan Liu, Sunnyvale, CA (US); Mehul Nariyawala, Los Altos, CA (US); Navneet Dalal, Atherton, CA (US)

(21) Appl. No.: 18/641,789

(22) Filed: Apr. 22, 2024

Related U.S. Application Data
(60) Provisional application No. 63/497,680, filed on Apr. 21, 2023.

(51) Int. Cl. A47L 7/00; A47L 9/12; A47L 9/14; A47L 9/28; A61L 9/12; A61L 11/00; C02F 1/50; G01F 23/292

(52) U.S. Cl. CPC A47L 7/0028; A47L 7/0042; A47L 9/12; A47L 9/1454; A47L 9/2805; A61L 9/12; A61L 11/00; C02F 1/50; G01F 23/2921; C02F 2303/04

(57) ABSTRACT

An autonomous vacuum implements a semi-waterproof waste bag capable of collecting dry waste from vacuum debris and liquid waste from mopping. The autonomous vacuum leverages a dual-roller cleaning head, one for dry cleaning and another for wet cleaning. The autonomous vacuum utilizes vacuum force to ingest both dry waste and liquid waste from the cleaning head into the semi-waterproof waste bag. The semi-waterproof waste bag includes a non-permeable portion formed of a waterproof material and a permeable portion formed of an air-permeable material. The semi-waterproof waste bag may further include a water-absorbent material, e.g., water-absorbing spherical beads.

### CROSS REFERENCE TO RELATED APPLICATIONS

This present application claims the benefit of and priority to U.S. Provisional Application No. 63/497,680 filed on Apr. 21, 2023, which is incorporated by reference in its entirety.

### TECHNICAL FIELD

This disclosure relates to autonomous cleaning systems. More particularly, this disclosure relates to a waste bag implemented in an autonomous vacuum.

### BACKGROUND

Traditional autonomous cleaning systems typically are single functional, i.e., focused separately on vacuuming or on mopping. Systems that attempt to combine the two functionalities, nonetheless, decouple components directed at each functionality, such that one subset of components focuses on the vacuuming, while another subset focuses on the mopping. The former systems might be efficient in their specific mode but are incapable of tackling messes of the other type. The latter systems typically sacrifice one cleaning function to promote the other. Either way, these systems are typically clunky incorporating two distinct sets of components. There remains a need for an efficient combinative system.

### BRIEF DESCRIPTION OF THE DRAWINGS

FIG. 1 is a block diagram of an autonomous vacuum, according to one or more embodiments.

FIG. 2 illustrates a spatial arrangement of components of the autonomous vacuum, according to one or more embodiments

FIG. 3 is a block diagram of a sensor system of the autonomous vacuum, according to one or more embodiments.

FIG. 4 is a block diagram of a controller of the autonomous vacuum, according to one or more embodiments.

FIG. 5 is a cross-sectional view of an airflow pathway for the autonomous vacuum, according to one or more embodiments.

FIG. 6 is a perspective view of a semi-waterproof waste bag for the autonomous vacuum, according to one or more embodiments.

FIG. 7 is a cross-sectional view of the semi-waterproof waste bag for the autonomous vacuum, according to one or more embodiments.

FIG. 8 is a cutaway perspective view of the semi-waterproof waste bag and the bag tab, according to one or more embodiments.

FIG. 9A is a perspective view of the waste bag removed from a bin cavity of the autonomous vacuum, according to one or more embodiments.

FIG. 9B is a perspective view of the waste bag installed into the bin cavity of the autonomous vacuum, according to one or more embodiments.

FIG. 9C is a perspective view of the bin cavity including a bag-bin seal implemented around a cavity opening, according to one or more embodiments.

FIG. 10A is a perspective view of the bag-bin seal, according to one or more embodiments.

FIG. 10B is a planar view of the bag-bin seal, according to one or more embodiments.

FIG. 10C is a cross-sectional view of the bag-bin seal, according to one or more embodiments.

FIG. 11A is a cross-sectional view of the bag tab of the waste bag interacting with the bag-bin seal of the bin cavity, according to one or more embodiments.

FIG. 11B is a cross-sectional view of the bag tab and the bag-bin seal when the waste bag is installed into the bin cavity, according to one or more embodiments.

FIG. 12A is a first close-up cross-sectional view of a top portion of the bag tab and the bag-bin seal, according to one or more embodiments.

FIG. 12B is a second close-up cross-sectional view of the top portion of the bag tab and the bag-bin seal, according to one or more embodiments.

FIG. 12C is a close-up cross-sectional view of a bottom portion of the bag tab and the bag-bin seal, according to one or more embodiments.

FIG. 13A is a first close-up cross-sectional view of the bag tab and the bag-bin seal, according to one or more embodiments.

FIG. 13B is a second close-up cross-sectional view of the bag tab and the bag-bin seal, according to one or more embodiments.

FIG. 13C is a third close-up cross-sectional view of the bag tab and the bag-bin seal, according to one or more embodiments.

FIG. 14A is an exploded view of the waste bag with a gasket coupled to the bag tab, according to one or more embodiments.

FIG. 14B is a constructed view of the waste bag with the gasket coupled to the bag tab, according to one or more embodiments.

FIG. 15 is a cross-sectional view of the waste bag with a plastic film for mitigation of condensation in the bin cavity, according to one or more embodiments.

FIG. 16A is an exploded view of the waste bag with the plastic film, according to one or more embodiments.

FIG. 16B is a perspective view of the waste bag with the plastic film showing spot welding locations, according to one or more embodiments.

FIG. 17 is a perspective view of the autonomous vacuum performing vacuum removal of humid air in the bin cavity, according to one or more embodiments.

FIG. 18 is a cross-sectional view of the autonomous vacuum with opened lid for venting of humid air in the bin cavity, according to one or more embodiments.

FIG. 19A is an exploded view of a lid of the autonomous vacuum with an elastic membrane, according to one or more embodiments.

FIG. 19B is a perspective view of the lid, according to one or more embodiments.

FIG. 20A is a cross-sectional view of the lid with the elastic membrane interacting with the bin cavity of the autonomous vacuum during idle time for venting humid air in the bin cavity, according to one or more embodiments.

FIG. 20B is a cross-sectional view of the lid with the elastic membrane interacting with the bin cavity of the autonomous vacuum during operation time, according to one or more embodiments.

FIG. 21 is a cross-sectional view of the waste bag choking the vacuum stack inlet in the bin cavity, according to one or more embodiments.

FIG. 22A is a perspective view of a lid with an anti-choking protrusion, according to one or more embodiments.

FIG. 22B is a cross-sectional view of the lid with the anti-choking protrusion to mitigate choking of the vacuum stack inlet by the waste bag, according to one or more embodiments.

FIG. 23A is a cross-sectional view of a modified waste bag with joined sections to mitigate choking of the vacuum stack inlet by the waste bag, according to one or more embodiments.

FIG. 23B is a perspective view of the modified waste bag with joined sections to mitigate choking of the vacuum stack inlet by the waste bag, according to one or more embodiments.

FIG. 24A is a cross-sectional view of the waste bag with water-absorbing beads, according to one or more embodiments.

FIG. 24B is a cross-sectional view of the waste bag with the water-absorbing beads in an expanded state upon water absorption, according to one or more embodiments.

FIG. 25A is a front perspective view of the waste bag, according to one or more embodiments.

FIG. 25B is a rear perspective view of the waste bag of FIG. 25A, according to one or more embodiments.

FIG. 25C is a front cutaway view of the waste bag of FIG. 25A, according to one or more embodiments.

FIG. 26 is a cross-sectional view of a solvent pathway in the autonomous vacuum, according to one or more embodiments.

FIG. 27 is a cross-sectional view of an optical sensor system for liquid waste fullness detection, according to one or more embodiments.

FIG. 28A is a cross-sectional view of a second optical sensor system for liquid waste fullness detection, according to one or more embodiments.

FIG. 28B is a cross-sectional view of the second optical sensor system for liquid waste fullness detection, according to one or more embodiments.

FIG. 29 is a cross-sectional view of a capacitive sensor system for liquid waste fullness detection, according to one or more embodiments.

FIG. 30 is a cross-sectional view of the airflow pathway in the autonomous vacuum with pressure port sensors, according to one or more embodiments.

FIG. 31 is a flowchart of dry waste fullness detection, according to one or more embodiments.

FIG. 32 is a flowchart of liquid waste fullness detection, according to one or more embodiments.

The figures depict various embodiments of the present invention for purposes of illustration only. One skilled in the art will readily recognize from the following discussion that alternative embodiments of the structures and methods illustrated herein may be employed without departing from the principles of the invention described herein.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### DETAILED DESCRIPTION

The Figures (FIGS.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### Configuration Overview

An autonomous vacuum with a semi-waterproof waste bag is capable of both vacuuming and mopping. The autonomous vacuum leverages a dual-roller cleaning head, one for dry cleaning and another for wet cleaning. The autonomous vacuum utilizes vacuum force to ingest both dry waste and liquid waste from the cleaning head into the semi-waterproof waste bag. The semi-waterproof waste bag generally includes a non-permeable portion formed of a waterproof material and a permeable portion formed of an air-permeable material. The semi-waterproof waste bag collects both the dry waste and the liquid waste. The semi-waterproof waste bag may further include a water-absorbent material, e.g., water-absorbing spherical beads.

When installed, the vacuum airflow is drawn through the permeable portion of the waste bag. The air is filtered through the permeable portion, such that contaminants are trapped in the permeable portion, while the filtered air continues to the vacuum stack inlet and exhausted out of the autonomous vacuum. To maintain high cleaning efficiency, the semi-waterproof waste bag forms an air-tight seal between the waste bag and the bin cavity of the autonomous vacuum which holds the waste bag. The autonomous vacuum may further include one or more condensation mitigation solutions, one or more anti-choking designs (e.g., to avoid the waste bag choking the vacuum stack inlet), or some combination thereof.

The autonomous vacuum leverages sensors and cleaning logic to detect fullness of the waste bag. The autonomous vacuum may include pressure sensors placed at different positions along the vacuum airflow pathway to measure strength of the vacuum force. This can inform when the dry contaminants have saturated the permeable portion, decreasing the air permeability. Below a certain threshold, the autonomous vacuum deems the waste bag to be dry-full. The autonomous vacuum may further include an optical sensor system for measuring liquid waste level in the waste bag. The optical sensor system may use a combination of light emitters and light receivers to track the liquid level in the waste bag. When the liquid level reaches a fill line, the autonomous vacuum can determine the waste bag to be wet-full. As the bag fills, the autonomous vacuum leverages fullness detection logic to identify when to prompt the user to change the waste bag.

### Autonomous Vacuum Architecture

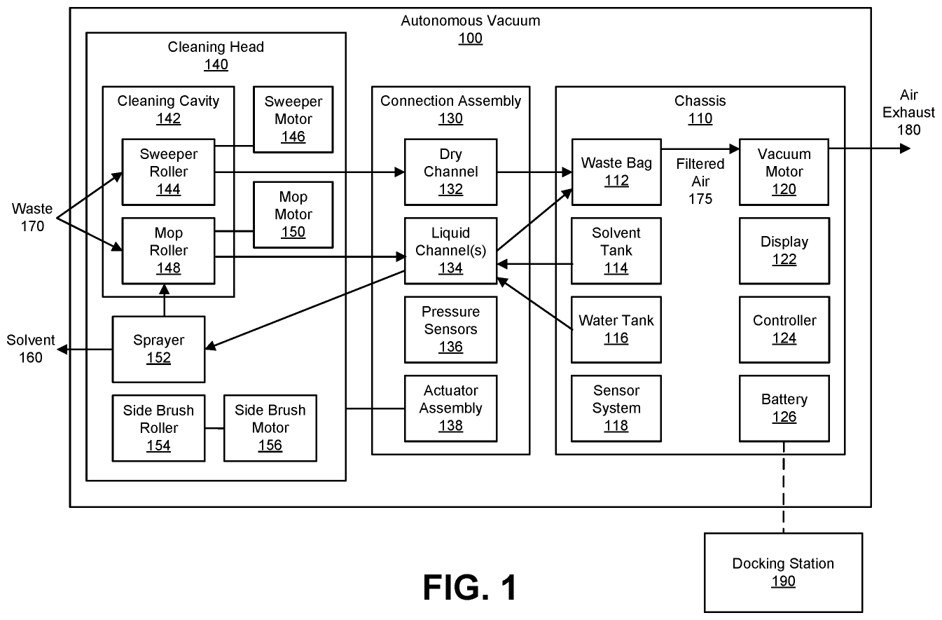

Figure (FIG. 1 is a block diagram of an autonomous vacuum 100, according to one or more embodiments. The autonomous vacuum 100 in this example may include a chassis 110, a connection assembly 130, and a cleaning head 140. The components of the autonomous vacuum 100 allow the autonomous vacuum 100 to intelligently clean as the autonomous vacuum 100 traverses an area within a physical environment.

As an overview, the chassis 110 is a rigid body that serves as a base frame for the autonomous vacuum. The chassis 110 comprises a plurality of motorized wheels for driving the autonomous vacuum 100. The chassis 110 hosts a suite of other components for navigating the autonomous vacuum 100, communicating with external devices, providing notifications, among other operations. The connection assembly 130 serves as a connection point between the cleaning head 140 and the chassis 110. The connection assembly 130 comprises at least a plurality of channels used to direct solvent, water, waste, or some combination thereof between the cleaning head 140 and the chassis 110. The connection assembly 130 also comprises an actuator assembly 138 that controls movement of the cleaning head 140. The cleaning head 140 comprises the one or more brush rollers used to perform cleaning operations. In some embodiments, the architecture of the autonomous vacuum 100 includes more components for autonomous cleaning purposes. Some examples include a mop roller, a solvent spray system, a waste container, and multiple solvent containers for different types of cleaning solvents. It is noted that the autonomous vacuum 100 may include functions that include cleaning functions that include, for example, vacuuming, sweeping, dusting, mopping, and/or deep cleaning.

The chassis 110 is a rigid body as a base frame for the autonomous vacuum 100. In one or more embodiments, the chassis 110 comprises at least a waste bag 112, a solvent tank 114, a water tank 116, a sensor system 118, a vacuum motor 120, a display 122, a controller 124, and a battery 126. In other embodiments, the chassis 110 may comprise additional, fewer, or different components than those listed herein. For example, one embodiment of the chassis 110 omits the display 122. Or another embodiment includes an additional output device, such as a speaker. Still another embodiment may combine the solvent tank 114 and the water tank 116 into a single tank.

The waste bag 112 collects the waste that is accumulated from performing cleaning routines. The waste bag 112 may be configured to collect solid and/or liquid waste. In one or more embodiments, there may be two separate waste bags, one for solid waste and one for liquid waste. The waste bag 112 may be removably secured within the chassis 110. As the waste bag 112 is filled, the autonomous vacuum 100 may alert the user to empty the waste bag 112 and/or replace the waste bag 112. In other embodiments, the waste bag 112 can remain in the chassis 110 when emptied. In such embodiments, the chassis 110 may further comprise a drainage channel connected to the waste bag to drain the collected waste. The waste bag 112 may further comprise an absorbent material that soaks up liquid, e.g., to prevent the liquid from sloshing out of the bag during operation of the autonomous vacuum 100.

The solvent tank 114 comprises solvent used for cleaning. The solvent tank 114 comprises at least a chamber and one or more valves for dispensing from the chamber. The solvent is a chemical formulation used for cleaning. Example solvents include dish detergent, soap, bleach, other organic and/or nonorganic solvents. In some embodiments, the solvent tank 114 comprises dry solvent that is mixed with water from the water tank 116 to create a cleaning solution. The solvent tank may be removable, allowing a user to refill the solvent tank 114 when the solvent tank 114 is empty.

The water tank 116 stores water used for cleaning. The water tank 116 comprises at least a chamber and one or more valves for dispensing from the chamber. The water tank 116 may be removable, allowing a user to refill the water tank 116 when the water tank 116 is empty. In one or more embodiments, the water tank 116 comprises a valve located on the bottom of the water tank 116, when the water tank 116 is secured in the chassis 110. The weight of the water applies a downward force due to gravity, a spring mechanism, or some combination thereof, which keeps the valve closed. To open the valve, some protrusion on the chassis 110 applies a counteracting upward force that opens the valve, e.g., by pushing the valve towards an interior of the chamber revealing an outlet permitting water to escape the water tank 116.

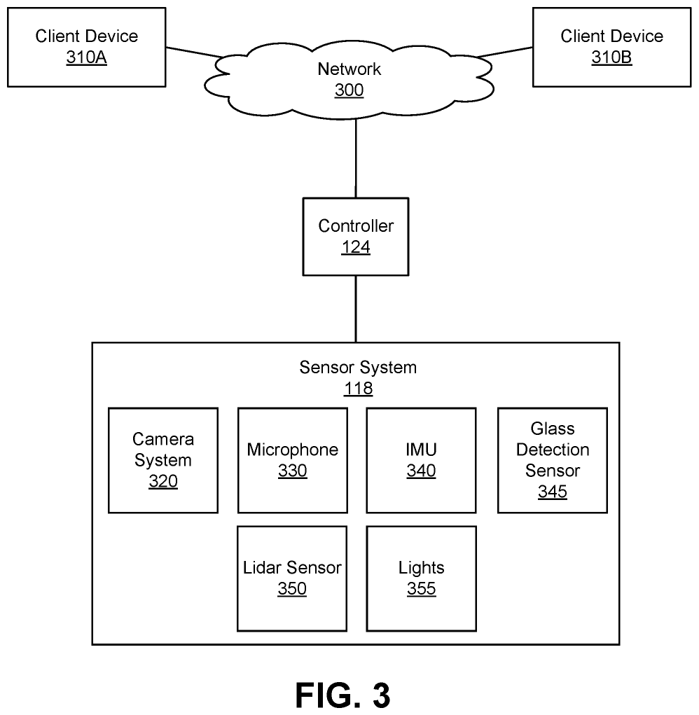

The sensor system 118 comprises a suite of sensors for guiding operation of the autonomous vacuum 100. Each sensor captures sensor data that can be fed to the controller 124 to guide operation of the autonomous vacuum 100. The sensor system 118 is further described in FIG. 3.

The vacuum motor 120 generates a vacuum force that aids ingestion of waste by the cleaning head 140. In one or more embodiments, to generate the vacuum force, the vacuum motor 120 may comprise one or more fans that rotate to rapidly move air. The airflow generated by vacuum force flows through the cleaning head 140, through the connection assembly 130, and the waste bag 112.

The display 122 is an electronic display that can display visual content. The display 122 may be positioned on a topside of the autonomous vacuum 100. The display may be configured to notify a user regarding operation of the autonomous vacuum 100. For example, notifications may describe an operation being performed by the autonomous vacuum 100, an error message, the health of the autonomous vacuum 100, etc. The display 122 may be an output device that includes a driver and/or screen to drive presentation of (e.g., provides for display) and/or present visual information. The display 122 may include an application programming interface (API) that allows users to interact with and control the autonomous vacuum. In some embodiments, the display may additionally or alternatively include physical interface buttons along with a touch sensitive interface. The display 122 receives data from the sensor system 118 and may display the data via the API. The data may include renderings of a view (actual image or virtual) of a physical environment, a route of the autonomous vacuum 100 in the environment, obstacles in the environment, and messes encountered in the environment. The data may also include alerts, analytics, and statistics about cleaning performance of the autonomous vacuum 100 and messes and obstacles detected in the environment.

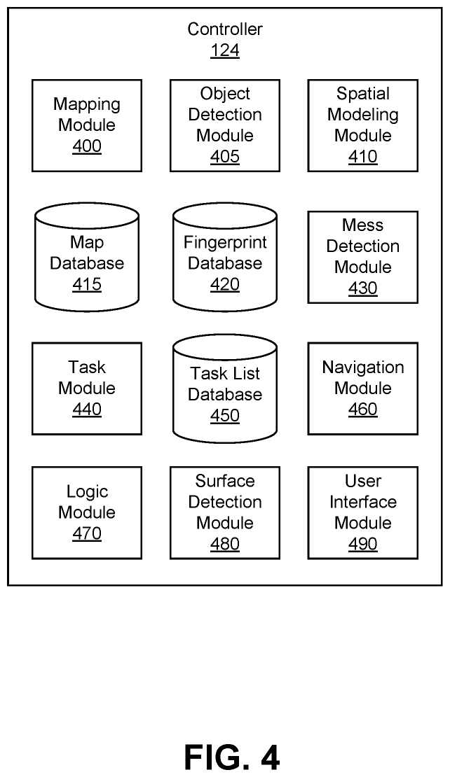

The controller 124 is a general computing device that controls operation of the autonomous vacuum 100. As a general computing device, the controller 124 may comprise at least one or more processors and computer-readable storage media for storing instructions executable by the processors. Operations of the controller 124 include navigating the autonomous vacuum 100, simultaneous localization and mapping of the autonomous vacuum 100, controlling operation of the cleaning head 140, generating notifications to provide to the user via one or more output devices (e.g., the display 122, a speaker, or a notification transmittable to the user's client device, etc.), running quality checks on the various components of the autonomous vacuum 100, controlling docking at the docking station 190, etc. Details of the controller 124 are described in FIG. 4.

The controller 124 may control movement of the autonomous vacuum 100. In particular, the controller connects to one or more motors connected to one or more wheels that may be used to move the autonomous vacuum 100 based on sensor data captured by the sensor system 118 (e.g., indicating a location of a mess to travel to). The controller may cause the motors to rotate the wheels forward/backward or turn to move the autonomous vacuum 100 in the environment. Based on surface type detection by the sensor system 118, the controller 124 may modify or alter navigation of the autonomous vacuum 100.

The controller of the actuator assembly 138 may also control cleaning operations. Cleaning operations may include a combination of rotation of the brush rollers, positioning or orienting the cleaning head 140 via the actuator assembly 138, controlling dispersion of solvent, activation of the vacuum motor 120, monitoring the sensor system 118, other functions of the autonomous vacuum, etc.

In controlling rotation of the brush rollers, the controller 124 may connect to one or more motors (e.g., the sweeper motor 146, the mop motor 150, and the side brush motor 156) positioned at the ends of the brush rollers. The controller 124 can toggle rotation of the brush rollers between rotating forward or backward or not rotating using the motors. In some embodiments, the brush rollers may be connected to an enclosure of the cleaning head 140 via rotation assemblies each comprising one or more of direct drive, geared or belted drive assemblies that connect to the motors to control rotation of the brush rollers. The controller 124 may rotate the brush rollers based on a direction needed to clean a mess or move a component of the autonomous vacuum 100. In some embodiments, the sensor system 118 determines an amount of pressure needed to clean a mess (e.g., more pressure for a stain than for a spill), and the controller 124 may alter the rotation of the brush rollers to match the determined pressure. The controller 124 may, in some instances, be coupled to a load cell at each brush roller used to detect pressure being applied by the brush roller. In another instance, the sensor system 118 may be able to determine an amount of current required to spin each brush roller at a set number of rotations per minute (RPM), which may be used to determine a pressure being exerted by the brush roller. The sensor system 118 may also determine whether the autonomous vacuum 100 is able to meet an expected movement (e.g., if a brush roller is jammed) and adjust the rotation via the controller if not. Thus, the sensor system 118 may optimize a load being applied by each brush roller in a feedback control loop to improve cleaning efficacy and mobility in the environment. The controller 124 may additionally control dispersion of solvent during the cleaning operation by controlling a combination of the sprayer 152, the liquid channels 134, the solvent tank 114, the water tank 116, and turning on/off the vacuum motor 120.

The battery 126 stores electric charge for powering the autonomous vacuum 100. The battery 126 may be rechargeable when the autonomous vacuum 100 is docked at the docking station 190. The battery 126 may implement a battery optimization scheme to efficiently distribute power across the various components. The autonomous vacuum 100 is powered with an internal battery 126. The battery 126 stores and supplies electrical power for the autonomous vacuum 100. In some embodiments, the battery 126 consists of multiple smaller batteries that charge specific components of the autonomous vacuum 100.

The autonomous vacuum 100 may dock at a docking station 190 to charge the battery 126. The docking station 190 may be connected to an external power source to provide power to the battery 126. External power sources may include a household power source and one or more solar panels. The docking station 190 also may include processing, memory, and communication computing components that may be used to communicate with the autonomous vacuum 100 and/or a cloud computing infrastructure (e.g., via wired or wireless communication). These computing components may be used for firmware updates and/or communicating maintenance status.

The docking station 190 also may include other components, such as a cleaning station for the autonomous vacuum 100. In some embodiments, the cleaning station includes a solvent tray that the autonomous vacuum 100 may spray solvent into and roll the roller 144 or the side brush roller 154 in for cleaning. In other embodiments, the autonomous vacuum may eject the waste bag 112 into a container located at the docking station 190 for a user to remove.

The connection assembly 130 is a rigid body that connects the cleaning head 140 to the chassis 110. A four-bar linkage may join the cleaning head 140 to the connection assembly 130. In one or more embodiments, the connection assembly 130 comprises at least a dry channel 132, one or more liquid channels 134, one or more pressure sensors 136, and an actuator assembly. Channel refers generally to either a dry channel or a liquid channel. In other embodiments, the connection assembly 130 may comprise additional, fewer, or different components than those listed herein. For example, one or more sensors of the sensor system 118 may be disposed on the connection assembly 130.

The dry channel 132 is a conduit for dry waste from the cleaning head 140 to the waste bag 112. The dry channel 132 is substantially large in diameter to permit movement of most household waste.

The one or more liquid channels 134 are conduits for liquids between the cleaning head 140 and the chassis 110. There is at least one liquid channel 134 (a liquid waste channel) that carries liquid waste from the cleaning head 140 to the waste bag 112. In one or more embodiments, the liquid channel 134 carrying liquid waste may be smaller in diameter than the dry channel 132. In such embodiments, the autonomous vacuum 100 sweeps (collecting dry waste) before mopping (collecting liquid waste). There is at least one other liquid channel 134 (a liquid solution channel) that carries water, solvent, and/or cleaning solution (combination of water and solvent) from the chassis 110 to the cleaning head 140 for dispersal to the cleaning environment.

The one or more pressure sensors 136 measure pressure in one or more of the channels. The pressure sensors 136 may be located at various positions along the connection assembly 130. The pressure sensors 136 provide the pressure measurements to the controller 124 for processing.

The actuator assembly 138 controls movement and position of the cleaning head 140, relative to the chassis 110. The actuator assembly 138 comprises at least one or more actuators configured to generate linear and/or rotational movement of the cleaning head 140. Linear movement may include vertical height of the cleaning head 140. Rotational movement may include pitching the cleaning head 140 to varying angles, e.g., to switch between sweeping mope and mopping mode, or to adjust cleaning by the cleaning head 140 based on detected feedback signals. The actuator assembly 138 may comprise a series of joints that aid in providing the movement to the cleaning head 140.

The cleaning head 140 performs one or more cleaning operations to clean an environment. The cleaning head 140 is also a rigid body that forms a cleaning cavity 142, where a sweeper roller 144 and a mop roller 148 are disposed. The cleaning head 140 further comprises a sweeper motor 146, a mop motor 150, a sprayer 152, a side brush roller 154, and a side brush motor 156. Collectively, the sweeper roller 144, the mop roller 148, and the side brush roller 154 are referred to as the brush rollers. Likewise, the brush motors include the sweeper motor 146, the mop motor 150, and the side brush motor 156. In some embodiments, each brush roller may be composed of different materials and operate at different times and/or speeds, depending on a cleaning task being executed by the autonomous vacuum 100. In other embodiments, the cleaning head 140 comprises additional, fewer, or different components than those listed herein. In some embodiments, the autonomous vacuum 100 may include two or more sweeper rollers 144 controlled by two or more sweeper motors 146. In some embodiments, the cleaning head 140 may be referred to as a roller housing.

The sweeper roller 144 sweeps dry waste into the autonomous vacuum 100. The sweeper roller 144 generally comprises one or more brushes attached to a cylindrical core. The sweeper roller 144 rotates to collect and clean messes. The sweeper roller 144 may be used to handle large particle messes, such as food spills or small plastic items like bottle caps. When the sweeper roller 144 is activated by the sweeper motor 146, the brushes act in concert to sweep dry waste towards a dry inlet connected to the dry channel 132. The brushes may be composed of a compliant material to sweep the dry waste. In some embodiments, the sweeper roller 144 may be composed of multiple materials for collecting a variety of waste, including, for example, synthetic bristle material, microfiber, wool, or felt.

The mop roller 148 mops the cleaning environment and ingests liquid waste into the autonomous vacuum 100. The mop roller 148 generally comprises fabric bristles attached to a cylindrical core. With the aid of a cleaning solution, the fabric bristles work to scrub away dirt, grease, or other contaminants that may have stuck to the cleaning surface. The mop motor 150 provides rotational force to the mop roller 148. In some embodiments, the mop roller 148 may be composed of multiple materials for collecting a variety of waste, including, for example, synthetic bristle material, microfiber, wool, or felt.

In normal sweeping mode, as the air flows from the dry channel 132 and the dry inlet towards the vacuum motor 120, the sweeper roller 144 rotates to move dry waste from the cleaning environment towards the inlet, in order to deposit the dry waste in the waste bag 112. In normal mopping mode, the cleaning head 140 sprays the cleaning solution (solvent or solvent mixed with water) onto the cleaning environment or on top of the mop roller itself. The mop roller 148 contacts the sprayed surface to scrub the surface with the fabric bristles. The vacuum force sucks up or ingests the liquid waste to deposit the liquid waste into the waste bag 112.

The side brush roller 154 sweeps dirt near a side of the cleaning head 140. The side brush roller 154 may rotate along an axis that is orthogonal or perpendicular to the ground. The side brush that is controlled by a side brush motor 156. The side brush roller 154 may be shaped like a disk or a radial arrangement of bristles that can push dirt into the path of the sweeper roller 144. In some embodiments, the side brush roller 154 is composed of different materials than the sweeper roller 144 to handle different types of waste and mess. In one or more embodiments, the side brush roller 154 may be concealed to minimize a profile of the cleaning head 140 when the side brush roller 154 is not in operation.

The sprayer 152 sprays liquid into the cleaning environment. The sprayer 152 is connected to the liquid solution channel that is connected to the solvent tank 114 and/or the water tank 116. A pump on the chassis 110 can dispense solvent and/or water from the solvent tank 114 and/or the water tank 116. The liquid travels to the sprayer 152, which then has a nozzle for spraying the liquid into the cleaning environment. The sprayer 152 may include a plurality of nozzles, e.g., two disposed on either side of the cleaning head.

The cleaning head 140 ingests waste 155 as the autonomous vacuum 100 cleans using the roller 144 and the side brush roller 154 and sends the waste 155 to the waste bag 112. The waste bag 112 collects and filters waste 155 from the air to send filtered air 165 out of the autonomous vacuum 100 through the vacuum motor 120 as air exhaust 170. The autonomous vacuum 100 may also use solvent 160 combined with pressure from the cleaning head 140 to clean a variety of surface types. The autonomous vacuum may dispense solvent 160 from the solvent tank 114 onto an area to remove dirt, such as dust, stains, and solid waste and/or clean up liquid waste. The autonomous vacuum 100 may also dispense solvent 160 into a separate solvent tray, which may be part of a charging station (e.g., docking station 190), described below, clean the roller 144 and the side brush roller 154.

Mess types are the form of mess in the environment, such as smudges, stains, and spills. It also includes the type of phase the mess embodies, such as liquid, solid, semi-solid, or a combination of liquid and solid. Some examples of waste include bits of paper, popcorn, leaves, and particulate dust. A mess typically has a size/form factor that is relatively small compared to obstacles that are larger. For example, spilled dry cereal may be a mess but the bowl it came in would be an obstacle. Spilled liquid may be a mess, but the glass that held it may be an obstacle. However, if the glass broke into smaller pieces, the glass would then be a mess rather than an obstacle. Further, if the sensor system 118 determines that the autonomous vacuum 100 cannot properly clean up the glass, the glass may again be considered an obstacle, and the sensor system 118 may send a notification to a user indicating that there is a mess that needs user cleaning. The mess may be visually defined in some embodiments, e.g., visual characteristics. In other embodiments it may be defined by particle size or make up. When defined by size, in some embodiments, a mess and an obstacle may coincide. For example, a small interlocking brick piece may be the size of both a mess and an obstacle. The sensor system 118 is further described in relation to FIG. 3.

The actuator assembly 138 includes one or more actuators (henceforth referred to as an actuator for simplicity), one or more controllers and/or processors (henceforth referred to as a controller for simplicity) that operate in conjunction with the sensor system 118 to control movement of the cleaning head 140. In particular, the sensor system 118 collects and uses sensor data to determine an optimal height for the cleaning head 140 given a surface type, surface height, and mess type. Surface types may be the floorings used in the environment and may include surfaces of varying characteristics (e.g., texture, material, absorbency), for example, carpet, wood, tile, rug, laminate, marble, and vinyl.

The actuator assembly 138 automatically adjusts the height of the cleaning head 140 given the surface type, surface height, and mess type. In particular, the actuator controls vertical movement and rotation tilt of the cleaning head 140. The actuator may vertically actuate the cleaning head 140 based on instructions from the sensor system 118. For example, the actuator may adjust the cleaning head 140 to a higher height if the sensor system 118 detects thick carpet in the environment than if the processor detects thin carpet. Further, the actuator may adjust the cleaning head 140 to a higher height for a solid waste spill than a liquid waste spill. In some embodiments, the actuator may set the height of the cleaning head 140 to push larger messes out of the path of the autonomous vacuum 100. For example, if the autonomous vacuum 100 is blocked by a pile of books, the sensor system 165 may detect the obstruction (i.e., the pile of books) and the actuator may move the cleanings head 105 to the height of the lowest book, and the autonomous vacuum 100 may move the books out of the way to continue cleaning an area. Furthermore, the autonomous vacuum 100 may detect the height of obstructions and/or obstacles, and if an obstruction or obstacle is over a threshold size, the autonomous vacuum 100 may use the collected visual data to determine whether to climb or circumvent the obstruction or obstacle by adjusting the cleaning head height using the actuator assembly 138.

In other embodiments, any of the components of the autonomous vacuum can be variable distributed among the chassis 110, the connection assembly 130, and the cleaning head 140.

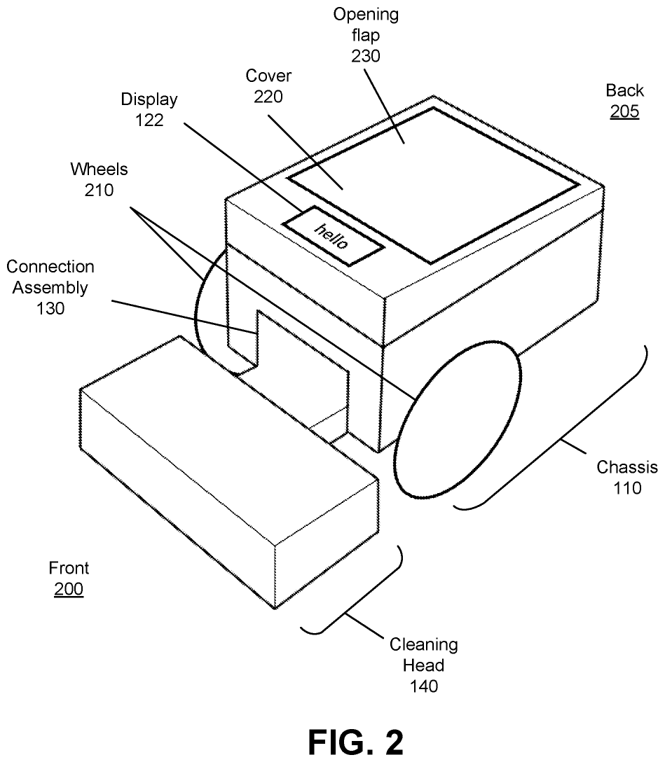

FIG. 2 illustrates a spatial arrangement of components of the autonomous vacuum 100, according to one or more embodiments. The autonomous vacuum 100 includes the cleaning head 140 (as described in relation to FIG. 1) at the front 200 and the chassis 110 at the back 205. The cleaning head 140 may be connected to the chassis 110 via the connection assembly 130 (e.g., a four-bar linkage system). The connection assembly 130 may be connected to one or more actuators of the actuator assembly 138 such that the actuators can control movement of the cleaning head 140 with the four-bar linkage system. Movement includes vertical translation of the cleaning head 140, i.e., towards or away from a ground plane, and rotation of the cleaning head 140, e.g., about a rotational axis established by a pivot point with the four-bar linkage system.

The chassis 110 includes the frame, a plurality of wheels 210, a cover 220, and opening flap 230, and a display 122. In particular, the cover 220 is an enclosed hollow structure that covers containers internal to the base that contain solvent and waste (e.g., in the waste bag 112). The opening flap 230 that may be opened or closed by a user such that the user can access the containers (e.g., to add more solvent, remove the waste bag 112, or put in a new waste bag 112). The cover may also house a subset of the sensors of the sensor system 118 and the actuator assembly 138, which may be configured at a front of the cover 220 to connect to the cleaning head 140. The display 122 is embedded in the cover 220 of the autonomous vacuum 100 and may include physical interface buttons and a touch sensitive interface.

### Sensor System

FIG. 3 is a block diagram of the sensor system 118 of the autonomous vacuum 100, according to one or more embodiments. The sensor system 118 receives sensor data from, for example, one or more cameras (video/visual), microphones 330 (audio), lidar sensors infrared (IR) sensors, and/or inertial sensors that capture inertial data (e.g., environmental surrounding or environment sensor data) about an environment for cleaning. The sensor system 118 uses the sensor data to map the environment and determine and execute cleaning tasks to handle a variety of messes. The controller 124 manages operations of the sensor system 118 and its various components. The controller 124 may communicate with one or more client devices 310 via a network 300 to send sensor data, alert a user to messes, or receive cleaning tasks to add to a task list.

The network 300 may comprise any combination of local area and/or wide area networks, using wired and/or wireless communication systems. In one embodiment, the network 300 uses standard communications technologies and/or protocols. For example, the network 300 includes communication links using technologies such as Ethernet, 802.11 (WiFi), worldwide interoperability for microwave access (WiMAX), 3G, 4G, 5G, code division multiple access (CDMA), digital subscriber line (DSL), Bluetooth, Near Field Communication (NFC), Universal Serial Bus (USB), or any combination of protocols. In some embodiments, all or some of the communication links of the network 300 may be encrypted using any suitable technique or techniques.

The client device 310 is a computing device capable of receiving user input as well as transmitting and/or receiving data via the network 300. Though only two client devices 310 are shown in FIG. 4, in some embodiments, more or less client devices 310 may be connected to the autonomous vacuum 100. In one embodiment, a client device 310 is a conventional computer system, such as a desktop or a laptop computer. Alternatively, a client device 310 may be a device having computer functionality, such as a personal digital assistant (PDA), a mobile telephone, a smartphone, a tablet, an Internet of Things (IoT) device, or another suitable device. A client device 310 is configured to communicate via the network 300. In one embodiment, a client device 310 executes an application allowing a user of the client device 310 to interact with the sensor system 118 to view sensor data, receive alerts, set cleaning settings, and add cleaning tasks to a task list for the autonomous vacuum 100 to complete, among other interactions. For example, a client device 310 executes a browser application with an application programming interface (API) that enables interactions between the client device 310 and the autonomous vacuum 100 via the network 300. In another embodiment, a client device 310 interacts with autonomous vacuum 100 through an application running on a native operating system of the client device 310, such as iOS® or ANDROID™.

In one or more embodiments, the sensor system 118 includes a camera system 320, microphone 330, inertial measurement device (IMU) 340, a glass detection sensor 345, a lidar sensor 350, and lights 355. The sensor system 118 may further include other sensors of the autonomous vacuum 100, e.g., the pressure sensors disposed in the connection assembly 130.

The camera system 320 comprises one or more cameras that capture images and or video signals as visual data about the environment. In some embodiments, the camera system 320 includes an IMU (separate from the IMU 340 of the sensor system 118) for capturing visual-inertial data in conjunction with the cameras. The visual data captured by the camera system 320 may be used by storage medium for image processing, as described in relation to FIG. 4.

The microphone 330 captures audio data by converting sound into electrical signals that can be stored or processed by other components of the sensor system 118. The audio data may be processed to identify voice commands for controlling functions of the autonomous vacuum 100, as described in relation to FIG. 4. In an embodiment, sensor system 118 uses more than one microphone 330, such as an array of microphones.

The IMU 340 captures inertial data describing the autonomous vacuum's 100 force, angular rate, and orientation. The IMU 340 may comprise of one or more accelerometers, gyroscopes, and/or magnetometers. In some embodiments, the sensor system 118 employs multiple IMUs 340 to capture a range of inertial data that can be combined to determine a more precise measurement of the autonomous vacuum's 100 position in the environment based on the inertial data.

The glass detection sensor 345 detects glass in the environment. Glass may be transparent material that may be stained, leaded, laminate or the like and may be part of furniture, flooring, or other objects in the environment (e.g., cups, mirrors, candlesticks, etc.). The glass detection sensor 345 may be an infrared sensor and/or an ultrasound sensor. In some embodiments, the glass detection sensor 345 is coupled with the camera system 320 to remove glare from the visual data when glass is detected. For example, the camera system 320 may have integrated polarizing filters that can be applied to the cameras of the camera system 320 to remove glare. In some embodiments, the glass sensor is a combination of an IR sensor and neural network that determines if an obstacle in the environment is transparent (e.g., glass) or opaque.

The lidar sensor 350 emits pulsed light into the environment and detects reflections of the pulsed light on objects (e.g., obstacles or obstructions) in the environment. Lidar data captured by the lidar sensor 350 may be used to determine a 3D representation of the environment. The lights 355 are one or more illumination sources that may be used by the autonomous vacuum 100 to illuminate an area around the autonomous vacuum 100. In some embodiments, the lights may be LEDs, e.g., having a static color such as white or green, or changeable colors (such as green of operating, red for stopped and yellow indicating slowing down).

The controller 124 may control the sensor system 118 various functions attributed to the sensor system 118 described herein. For example, a storage medium may store one or more modules or applications (described in relation to FIG. 4) embodied as instructions executable by a processor. The instructions, when executed by the processor, cause the processor to carry out the functions attributed to the various modules or applications described herein or instruct the controller and/or actuator to carry out movements and/or functions. For example, instructions may include when to take the sensor data, where to move the autonomous vacuum 100 to, and how to clean up a mess. In one embodiment, the processor may comprise a single processor or a multi-processor system.

### Controller Architecture

FIG. 4 is a block diagram of the controller 124, according to one or more embodiments. The controller 124 includes a mapping module 400, an object detection module 405, a spatial modeling module 410, a map database 415, a fingerprint database 420, a mess detection module 430, a task module 440, a task list database 450, a navigation module 460, a logic module 470, a surface detection module 480, and a user interface module 490. In some embodiments, the controller 124 includes other modules that control various functions for the autonomous vacuum 100. Examples include a separate image processing module, a separate command detection module, and an object database.

The mapping module 400 creates and updates a map of an environment as the autonomous vacuum 100 moves around the environment. The map may be a two-dimensional (2D) or a three-dimensional (3D) representation of the environment including objects and other defining features in the environment. For simplicity, the environment may be described in relation to a house in this description, but the autonomous vacuum 100 may be used in other environments in other embodiments. Example environments include offices, retail spaces, and classrooms. For a first mapping of the environment, the mapping module 400 receives visual data from the camera system 320 and uses the visual data to construct a map. In some embodiments, the mapping module 400 also uses inertial data from the IMU 340 and lidar and IR data to construct the map. For example, the mapping module 400 may use the inertial data to determine the position of the autonomous vacuum 100 in the environment, incrementally integrate the position of the autonomous vacuum 100, and construct the map based on the position. However, for simplicity, the data received by the mapping module 400 will be referred to as visual data throughout the description of this figure.

In another embodiment, the mapping module 400 may capture a 360 degree “panorama view” using the camera system 320 while the autonomous vacuum 100 rotates around a center axis. The mapping module 400 applies a neural network to the panorama view to determine a boundary within the environment (e.g., walls), which the mapping module 400 may use for the representation of the environment. In other embodiments, the mapping module 400 may cause the autonomous vacuum 100 to trace the boundary of the environment by moving close to walls or other bounding portions of the environment using the camera system 100. The mapping module 400 uses the boundary for the representation.

In another embodiment, mapping module 400 may use auto-detected unique key points and descriptions of these key points to create a nearest neighborhood database in the map database 410. Each key point describes a particular feature of the environment near the autonomous vacuum 100 and the descriptions describe aspects of the features, such as color, material, location, etc. As the autonomous vacuum 100 moves about the environment, the mapping module 400 uses the visual data to extract unique key points and descriptions from the environment.

In some embodiments, the mapping module 400 may determine key points using a neural network. The mapping module 400 estimates which key points are visible in the nearest neighborhood database by using the descriptions as matching scores. After the mapping module 400 determines there are a threshold number of key points within visibility, the mapping module 400 uses these key points to determine a current location of the autonomous vacuum 100 by triangulating the locations of the key points with both the image location in the current visual data and the known location (if available) of the key point from the map database 415.

In another embodiment, the mapping module 400 uses the key points between a previous frame and a current frame in the visual data to estimate the current location of the autonomous vacuum 100 by using these matches as reference. This is typically done when the autonomous vacuum 100 is seeing a new scene for the first time, or when the autonomous vacuum 100 is unable to localize using previously existing key points on the map. Using this embodiment, the mapping module 400 can determine the position of the autonomous vacuum 100 within the environment at any given time. Further, the mapping module 400 may periodically purge duplicate key points and add new descriptions for key points to consolidate the data describing the key points. In some embodiments, this is done while the autonomous vacuum 100 is at the docking station 190.

The mapping module 400 processes the visual data when the autonomous vacuum 100 is at the docking station 190. The mapping module 400 runs an expansive algorithm to process the visual data to identify the objects and other features of the environment and piece them together into the map. The mapping module stores the map in the map database 415 and may store the map as a 3D satellite view of the environment. The mapping module 400 may update the map in the map database 415 to account for movement of objects in the environment upon receiving more visual data from the autonomous vacuum 100 as it moves around the environment over time. By completing this processing at the docking station, the autonomous vacuum 100 may save processing power relative to mapping and updating the map while moving around the environment. The mapping module 400 may use the map to quickly locate and/or determine the location of the autonomous vacuum 100 within the environment, which is faster than when computing the map at the same time. This allows the autonomous vacuum 100 to focus its processing power while moving on mess detection, localization, and user interactions while saving visual data for further analysis at the docking station 190.

The mapping module 400 constructs a layout of the environment as the basis of the map using visual data. The layout may include boundaries, such as walls, that define rooms, and the mapping module 400 layers objects into this layout to construct the map. In some embodiments, the mapping module 400 may use surface normals from 3D estimates of the environment and find dominant planes using one or more algorithms, such as RANSAC, which the mapping module 400 uses to construct the layout. In other embodiments, the mapping module 400 may predict masks corresponding to dominant planes in the environment using a neural network trained to locate the ground plane and surface planes on each side of the autonomous vacuum 100. If such surface planes are not present in the environment, the neural network may output an indication of “no planes.” The neural network may be a state-of-the-art object detection and masking network trained on a dataset of visual data labeled with walls and other dominant planes.

The mapping module 400 also uses the visual data to analyze surfaces throughout the environment. The mapping module 400 may insert visual data for each surface into the map to be used by the mess detection module 430 as it detects messes in the environment, described further below. For each different surface in the environment, the mapping module 400 determines a surface type of the surface and tags the surface with the surface type in the map. Surface types include various types of carpet, wood, tile, and cement, and, in some embodiments, the mapping module 400 determines a height for each surface type. For example, in a house, the floor of a dining room may be wood, the floor of a living room may be nylon carpet, and the floor of a bedroom may be a polyester carpet that is thicker than a nylon carpet in a hallway. The mapping module may also determine and tag surface types for objects in the room, such as carpets or rugs. The mapping module 400 sends the information about the surface types in the environment to the surface detection module 480.

The mapping module 400 further analyzes the visual data to determine the objects in the environment. Objects may include furniture, rugs, people, pets, and everyday household objects that the autonomous vacuum 100 may encounter on the ground, such as books, toys, and bags. Some objects may be barriers that define a room or obstacles that the autonomous vacuum 100 may need to remove, move, or go around, such as a pile of books. To identify the objects in the environment, the mapping module 400 predicts the plane of the ground in the environment using the visual data and removes the plane from the visual data to segment out an object in 3D.

In some embodiments, the mapping module 400 uses an object database to determine what an object is. In other embodiments, the mapping module 400 retrieves and compares visual data retrieved from an external server to the segmented objects to determine what the object is and tag the object with a descriptor. In further embodiments, the mapping module 400 may use the pretrained object detection module 405, which may be neural network based, to detect and pixel-wise segment objects such as chairs, tables, books, shoes. For example, the mapping module 400 may tag each of 4 chairs around a table as “chair” and the table as “table” and may include unique identifiers for each object (i.e., “chair A” and “chair B”).

In some embodiments, the mapping module 400 may also associate or tag an object with a barrier or warning. For example, the mapping module 400 may construct a virtual border around the top of a staircase in the map such that the autonomous vacuum 100 does not enter the virtual border to avoid falling down the stairs. As another example, the mapping module 400 may tag a baby with a warning that the baby is more fragile than other people in the environment.

The map may include three distinct levels for the objects in the environment: a long-term level, an intermediate level, and an immediate level. Each level may layer onto the layout of the environment to create the map of the entire environment. The long-term level contains a mapping of objects in the environment that are static. In some embodiments, an object may be considered static if the autonomous vacuum 100 has not detected that the object moved within the environment for a threshold amount of time (e.g., 10 days or more). In other embodiments, an object is static if the autonomous vacuum 100 never detects that the object moved. For example, in a bedroom, the bed may not move locations within the bedroom, so the bed would be part of the long-term level. The same may apply for a dresser, a nightstand, or an armoire. The long-term level also includes fixed components of the environment, such as walls, stairs, or the like.

The intermediate level contains a mapping of objects in the environment that are dynamic. These objects move regularly within the environment and may be objects that are usually moving, like a pet or child, or objects that move locations on a day-to-day basis, like chairs or bags. The mapping module 400 may assign objects to the intermediate level upon detecting that the objects move more often than a threshold amount of time. For example, the mapping module 400 may map chairs in a dining room to the intermediate level because the chairs move daily on average, but map the dining room table to the long-term level because the visual data has not shown that the dining room table has moved in more than 5 days. However, in some embodiments, the mapping module 400 does not use the intermediate level and only constructs the map using the long-term level and the immediate level.

The immediate level contains a mapping of objects within a threshold radius of the autonomous vacuum 100. The threshold radius may be set at a predetermined distance (i.e., 5 feet) or may be determined based on the objects the autonomous vacuum 100 can discern using the camera system 320 within a certain resolution given the amount of light in the environment. For example, the immediate level may contain objects in a wider vicinity around the autonomous vacuum 100 around noon, which is a bright time of day, than in the late evening, which may be darker if no indoor lights are on. In some embodiments, the immediate level includes any objects within a certain vicinity of the autonomous vacuum 100.

In other embodiments, the immediate level only includes objects within a certain vicinity that are moving, such as people or animals. For each person within the environment, the mapping module 400 may determine a fingerprint of the person to store in the fingerprint database 420. A fingerprint is a representation of a person and may include both audio and visual information, such as an image of the person's face (i.e., a face print), an outline of the person's body (i.e., a body print), a representation of the clothing the person is wearing, and a voice print describing aspects of the person's voice determined through voice print identification. The mapping module 400 may update a person's fingerprint in the fingerprint database 420 each time the autonomous vacuum 400 encounters the person to include more information describing the person's clothing, facial structure, voice, and any other identifying features.

In another embodiment, when the mapping module 400 detects a person in the environment, the mapping module 400 creates a temporary fingerprint using the representation of the clothing the person is currently wearing and uses the temporary fingerprint to track and follow a person in case this person interacts with the autonomous vacuum 100, for example, by telling the autonomous vacuum 100 to “follow me.” Embodiments using temporary fingerprints allow the autonomous vacuum 100 to track people in the environment even without visual data of their faces or other defining characteristics of their appearance.

The mapping module 400 updates the mapping of objects within these levels as it gathers more visual data about the environment over time. In some embodiments, the mapping module 400 only updates the long-term level and the intermediate level while the autonomous vacuum 100 is at the docking station, but updates immediate level as the autonomous vacuum 100 moves around the environment. For objects in the long-term level, the mapping module 400 may determine a probabilistic error value about the movement of the object indicating the chance that the object moved within the environment and store the probabilistic error value in the map database 415 in association with the object. For objects in the long-term map with a probabilistic error value over a threshold value, the mapping module 400 characterizes the object in the map and an area that the object has been located in the map as ambiguous.

In some embodiments, the object detection module 405 detects and segments various objects in the environment. Some examples of objects include tables, chairs, shoes, bags, cats, and dogs. In one embodiment, the object detection module 405 uses a pre-trained neural network to detect and segment objects. The neural network may be trained on a labeled set of data describing an environment and objects in the environment. The object detection module 405 also detects humans and any joint points on them, such as knees, hips, ankles, wrists, elbows, shoulders, and head. In one embodiment, the object detection module 405 determines these joint points via a pre-trained neural network system on a labeled dataset of humans with joint points.

In some embodiments, the mapping module 400 uses the optional spatial modeling module 410 to create a 3D rendering of the map. The spatial modeling module 410 uses visual data captured by stereo cameras on the autonomous vacuum 100 to create an estimated 3D rendering of a scene in the environment. In one embodiment, the spatial modeling module 410 uses a neural network with an input of two left and right stereo images and learns to produce estimated 3D renderings of videos using the neural network. This estimated 3D rendering can then be used to find 3D renderings of joint points on humans as computed by the object detection module 405. In one embodiment, the estimated 3D rendering can then be used to predict the ground plane for the mapping module 400. To predict the ground plane, the spatial modeling module 410 uses a known camera position of the stereo cameras (from the hardware and industrial design layout) to determine an expected height ground plane. The spatial modeling module 410 uses all image points with estimated 3D coordinates at the expected height as the ground plane. In another embodiment, the spatial modeling module 410 can use the estimated 3D rendering to estimate various other planes in the environment, such as walls. To estimate which image points are on a wall, the spatial modeling module 410 estimates clusters of image points that are vertical (or any expected angle for other planes) and groups connected image points into a plane.

In some embodiments, the mapping module 400 passes the 3D rendering through a scene-classification neural network to determine a hierarchical classification of the home. For example, a top layer of the classification decomposes the environment into different room types (e.g., kitchen, living room, storage, bathroom, etc.). A second layer decomposes each room according to objects (e.g., television, sofa, and vase in the living room and bed, dresser, and lamps in the bedroom). The autonomous vacuum 100 may subsequently use the hierarchical model in conjunction with the 3D rendering to understand the environment when presented with tasks in the environment (e.g., “clean by the lamp”). It is noted that the map ultimately may be provided for rendering on a device (e.g., wirelessly or wired connected) with an associated screen, for example, a smartphone, tablet, laptop or desktop computer. Further, the map may be transmitted to a cloud service before being provided for rendering on a device with an associated screen.

The mess detection module 430 detects messes within the environment, which are indicated by pixels in real-time visual data that do not match the surface type. As the autonomous vacuum 100 moves around the environment, the camera system 320 collects a set of visual data about the environment and sends it to the mess detection module 430. From the visual data, the mess detection module 430 determines the surface type for an area of the environment, either by referencing the map or by comparing the collected visual data to stored visual data from a surface database. In some embodiments, the mess detection module 430 may remove or disregard objects other than the surface in order to focus on the visual data of the ground that may indicate a mess. The mess detection module 430 analyzes the surface in the visual data pixel-by-pixel for pixels that do not match the pixels of the surface type of the area. For areas with pixels that do not match the surface type, the mess detection module 430 segments out the area from the visual data using a binary mask and compares the segmented visual data to the long-term level of the map. In some embodiments, since the lighting when the segmented visual data was taken may be different from the lighting of the visual data in the map, the mess detection module 430 may normalize the segmented visual data for the lighting. For areas within the segmented visual data where the pixels do not match the map, the mess detection module 430 flags the area as containing a mess and sends the segmented visual data, along with the location of the area on the map, to the task module 440, which is described below. In some embodiments, the mess detection module 430 uses a neural network for detecting dust in the segmented visual data.

For each detected mess, the mess detection module 430 verifies that the surface type for the area of the mess matches the tagged surface type in the map for that area. In some embodiments, if the surface types do not match to within a confidence threshold, the mess detection module 430 re labels the surface in the map with the newly detected surface type. In other embodiments, the mess detection module 430 requests that the autonomous vacuum 100 collect more visual data to determine the surface type to determine the surface type of the area.

The mess detection module 430 may also detect messes and requested cleaning tasks via user interactions from a user in the environment. As the autonomous vacuum 100 moves around the environment, the sensor system 118 captures ambient audio and visual data using the microphone 330 and the camera system 320 that is sent to the mess detection module 430. In one embodiment, where the microphone 330 is an array of microphones 330, the detection module 330 may process audio data from each of the microphones 330 in conjunction with one another to generate one or more beamformed audio channels, each associated with a direction (or, in some embodiments, range of directions). In some embodiments, the mess detection module 430 may perform image processing functions on the visual data by zooming, panning, de-warping.

When the autonomous vacuum 100 encounters a person in the environment, the mess detection module 430 may use face detection and face recognition on visual data collected by the camera system 320 to identify the person and update the person's fingerprint in the fingerprint database 440. The mess detection module 430 may use voice print identification on a user speech input a person (or user) to match the user speech input to a fingerprint and move to that user to receive further instructions. Further, the mess detection module 430 may parse the user speech input for a hotword that indicates the user is requesting an action and process the user speech input to connect words to meanings and determine a cleaning task. In some embodiments, the mess detection module 430 also performs gesture recognition on the visual data to determine the cleaning task. For example, a user may ask the autonomous vacuum 100 to “clean up that mess” and point to a mess within the environment. The mess detection module 430 detects and processes this interaction to determine that a cleaning task has been requested and determines a location of the mess based on the user's gesture. To detect the location of the mess, the mess detection module 430 obtains visual data describing the user's hands and eyes from the object detection module 405 and obtains an estimated 3D rendering of the user's hands and eyes from spatial modeling module 410 to create a virtual 3D ray. The mess detection module 430 intersects the virtual 3D ray with an estimate of the ground plane to determine the location the user is pointing to. The detection module 440 sends the cleaning task (and location of the mess) to the task module 440 to determine a mess type, surface type, actions to remove the mess, and cleaning settings, described below.

In some embodiments, the mess detection module 430 may apply a neural network to visual data of the environment to detect dirt in the environment. In particular, the mess detection module 430 may receive real-time visual data captured by the sensor system 118 (e.g., camera system and/or infrared system) and input the real-time visual data to the neural network. The neural network outputs a likelihood that the real-time visual data includes dirt, and may further output likelihoods that the real-time visual data includes dust and/or another mess type (e.g., a pile or spill) in some instances. For each of the outputs from the neural network, if the likelihood for any mess type is above a threshold, the mess detection module 430 flags the area as containing a mess (i.e., an area to be cleaned).

The mess detection module 430 may train the neural network on visual data of floors. In some embodiments, the mess detection module 430 may receive a first set of visual data from the sensor system 118 of an area in front of the autonomous vacuum 100 and a second set of visual data of the same area from behind the autonomous vacuum 100 after the autonomous vacuum 100 has cleaned the area. The autonomous vacuum 100 can capture the second set of visual data using cameras on the back of the autonomous vacuum or by turning around to capture the visual data using cameras on the front of the autonomous vacuum. The mess detection module 430 may label the first and second sets of visual data as “dirty” and “clean,” respectively, and train the neural network on the labeled sets of visual data. The mess detection module 430 may repeat this process for a variety of areas in the environment to train the neural network for the particular environment or for a variety of surface and mess types in the environment.

In another embodiment, the mess detection module 430 may receive visual data of the environment as the autonomous vacuum 100 clean the environment. The mess detection module 430 may pair the visual data to locations of the autonomous vacuum 100 determined by the mapping module 400 as the autonomous vacuum moved to clean. The mess detection module 430 estimates correspondence between the visual data to pair visual data of the same areas together based on the locations. The mess detection module 430 may compare the paired images in the RGB color space (or any suitable color or high-dimensional space that may be used to compute distance) to determine where the areas were clean or dirty and label the visual data as “clean” or “dirty” based on the comparison. Alternatively, the mess detection module 430 may compare the visual data to the map of the environment or to stored visual data for the surface type shown in the visual data. The mess detection module 430 may analyze the surface in the visual data pixel-by-pixel for pixels that do not match the pixels of the surface type of the area and label pixels that do not match as “dirty” and pixels that do match as “clean.” The mess detection module 430 trains the neural network on the labeled visual data to detect dirt in the environment.

In another embodiment, the mess detection module 430 may receive an estimate of the ground plane for a current location in the environment from the spatial modeling module 410. The mess detection module 430 predicts a texture of the floor of the environment based on the ground plane as the autonomous vacuum 100 moves around and labels visual data captured by the autonomous vacuum 100 with the floor texture predicted while the autonomous vacuum 100 moves around the environment. The mess detection module 430 trains the neural network on the labeled visual data to predict if a currently predicted floor texture maps to a previously predicted floor texture. The mess detection module 430 may then apply the neural network to real-time visual data and a currently predicted floor texture, and if the currently predicted floor texture does not map a previously predicted floor texture, the mess detection module 430 may determine that the area being traversed is dirty.

The task module 440 determines cleaning tasks for the autonomous vacuum 100 based on user interactions and detected messes in the environment. The task module 440 receives segmented visual data from the mess detection module 430 the location of the mess from the mess detection module 430. The task module 440 analyzes the segmented visual data to determine a mess type of the mess. Mess types describe the type and form of waste that comprises the mess and are used to determine what cleaning task the autonomous vacuum 100 should do to remove the mess. Examples of mess types include a stain, dust, a liquid spill, a solid spill, a smudge, or another type of debris. The mess types may include liquid waste, solid waste, or a combination of liquid and solid waste.

The task module 440 retrieves the surface type for the location of the mess from the map database and matches the mess type and surface type to a cleaning task architecture that describes the actions for the autonomous vacuum 100 to take to remove the mess. In some embodiments, the task module 440 uses a previous cleaning task from the task database for the given mess type and surface type. In other embodiments, the task module 440 matches the mess type and surfaces to actions the autonomous vacuum 100 can take to remove the mess and creates a corresponding cleaning task architecture of an ordered list of actions. In some embodiments, the task module 440 stores a set of constraints that it uses to determine cleaning settings for the cleaning task. The set of constraints describe what cleaning settings cannot be used for each mess type and surface type and how much force to apply to clean the mess based on the surface type. Cleaning settings include height of the cleaning head 140 and rotation speed of the brush roller 135 and the use of solvent 160. For example, the set of constraints may indicate that the solvent 160 can be used on wood and tile, but not on carpet, and the height of the cleaning head 140 must be at no more than 3 centimeters off the ground for cleaning stains in the carpet but at least 5 centimeters and no more than 7 centimeters off the ground to clean solid waste spills on the carpet.

Based on the determined actions and the cleaning settings for the mess, the task module 440 adds a cleaning task for each mess to task list database 450. The task list database 450 stores the cleaning tasks in a task list. The task list database 450 may associate each cleaning task with a mess type, a location in the environment, a surface type, a cleaning task architecture, and cleaning settings. For example, the first task on the task list in the task list database 450 may be a milk spill on tile in a kitchen, which the autonomous vacuum 100 may clean using solvent 160 and the brush roller 135. The cleaning tasks may be associated with a priority ranking that indicates how to order the cleaning tasks in the task list. In some embodiments, the priority ranking is set by a user via a client device 310 or is automatically determined by the autonomous vacuum 100 based on the size of the mess, the mess type, or the location of the mess. For example, the autonomous vacuum 100 may prioritize cleaning tasks in heavily trafficked areas of the environment (i.e., the living room of a house over the laundry room) or the user may rank messes with liquid waste with a higher priority ranking than messes with solid waste.

In some embodiments, the task module 440 adds cleaning tasks to the task list based on cleaning settings entered by the user. The cleaning settings may indicate which cleaning tasks the user prefers the autonomous vacuum 100 to complete on a regular basis or after a threshold amount of time has passed without a mess resulting in that cleaning task occurring. For example, the task module 440 may add a carpet deep cleaning task to the task list once a month and a hard wood polishing task to the task list if the hard wood has not been polished in more than some predetermined time period, e.g., 60 days.

The task module 440 may add additional tasks to the task list if the autonomous vacuum 100 completes all cleaning tasks on the task list. Additional tasks include charging at the docking station 190, processing visual data for the map via the mapping module 400 at the docking station 190, which may be done in conjunction with charging, and moving around the environment to gather more sensor data for detecting messes and mapping. The task module 440 may decide which additional task to add to the task list based on the amount of charge the battery 126 has or user preferences entered via a client device 310.

The task module 440 also determines when the autonomous vacuum 100 needs to be charged. If the task module 440 receives an indication from the battery 126 that the battery is low on power, the task module adds a charging task to the task list in the task list database 450. The charging task indicates that the autonomous vacuum 100 should navigate back to the docking station 190 and dock for charging. In some embodiments, the task module 440 may associate the charging task with a high priority ranking and move the charging task to the top of the task list. In other embodiments, the task module 440 may calculate how much power is required to complete each of the other cleaning tasks on the task list and allow the autonomous vacuum 100 to complete some of the cleaning tasks before returning to the docking station 190 to charge.

The navigation module 460 determines the location of the autonomous vacuum 100 in the environment. Using real-time sensor data from the sensor system 118, the navigation module 460 matches the visual data of the sensor data to the long-term level of the map to localize the autonomous vacuum 100. In some embodiments, the navigation module 460 uses a computer vision algorithm to match the visual data to the long-term level. The navigation module 460 sends information describing the location of the autonomous vacuum 100 to other modules within the controller 124. For example, the mess detection module 430 may use the location of the autonomous vacuum 100 to determine the location of a detected mess.

The navigation module 460 uses the immediate level of the map to determine how to navigate the environment to execute cleaning tasks on the task list. The immediate level describes the locations of objects within a certain vicinity of the autonomous vacuum 100, such as within the field of view of each camera in the camera system 320. These objects may pose as obstacles for the autonomous vacuum 100, which may move around the objects or move the objects out of its way. The navigation module interlays the immediate level of the map with the long-term level to determine viable directions of movement for the autonomous vacuum 100 based on where objects are not located. The navigation module 460 receives the first cleaning task in the task list database 450, which includes a location of the mess associated with the cleaning task. Based on the location determined from localization and the objects in the immediate level, the navigation module 100 determines a path to the location of the mess. In some embodiments, the navigation module 460 updates the path if objects in the environment move while the autonomous vacuum 100 is in transit to the mess. Further, the navigation module 460 may set the path to avoid fragile objects in the immediate level (e.g., a flower vase or expensive rug).

The logic module 470 determines instructions for the controller 124 to control the autonomous vacuum 100 based on the map in the map database 415, the task list database 450, and the path and location of the autonomous vacuum 100 determined by the navigation module 460. The instructions describe what each physical feature of the autonomous vacuum 100 should do to navigate an environment and execute tasks on the task list. Some of the physical features of the autonomous vacuum 100 include the brush motor 140, the side brush motor 150, the solvent pump 175, the actuator assembly 138, the vacuum pump 115, and the wheels 210. The logic module 470 also controls how and when the sensor system 118 collects sensor data in the environment. For example, logic module 470 may receive the task list from the task list database 450 and create instructions on how to navigate to handle the first cleaning task on the task list based on the path determined by the navigation module, such as rotating the wheels 210 or turning the autonomous vacuum 100. The logic module may update the instructions if the navigation module 460 updates the path as objects in the environment moved. Once the autonomous vacuum 100 has reached the mess associated with the cleaning task, the logic module 470 may generate instructions for executing the cleaning task. These instructions may dictate for the actuator assembly 138 to adjust the cleaning head height, the vacuum pump 115 to turn on, the brush roller 135 and/or side brush roller 154 to rotate at certain speeds, and the solvent motor 120 to dispense an amount of solvent 160, among other actions for cleaning. The logic module 470 may remove the cleaning task from the task list once the cleaning task has been completed and generate new instructions for the next cleaning task on the task list.

Further, the logic module 470 generates instructions for the controller 124 to execute. The instructions may include internal instructions, such as when to tick a clock node or gather sensor data, or external instructions, such as controlling the autonomous vacuum 100 to execute a cleaning task to remove a mess. The logic module 470 may retrieve data describing the map of the environment stored in the map database 415, fingerprint database 420, and task list database 450, or from other modules in the controller 124, to determine these instructions. The logic module 470 may also receive alerts/indications from other components of the autonomous vacuum 100 or from an external client device 310 that it uses to generate instructions for the controller 124. The logic module 470 may also send sensor data to one or more of the modules and send indications to the one or more modules when the autonomous vacuum is traversing in the environment, a real-time location of the autonomous vacuum 100, and the like.

The surface detection module 480 determines characteristics of surface types in an environment. Characteristics of a surface type may include, for example, material (e.g., marble, ceramic, oak, nylon, etc.), placement pattern (e.g., stacked squares, interlocking rectangles, etc.), grain/weave (e.g., density, visual texture and color variation), texture (shag, glossy, etc.), and color. The surface detection module 480 may determine the characteristics of the surface types of the environment in real-time or based on visual-inertial data saved in relation to the map of the environment.

In some embodiments, the surface detection module 480 receives real-time visual-inertial data of an environment from the logic module 470 as the autonomous vacuum 100 traverses the environment. The surface detection module 480 may also receive a real-time location of the autonomous vacuum in the environment from the logic module 470 and retrieve a surface type of the location based on the map stored in the map database 415. In other embodiments, the surface detection module 480 accesses visual-inertial data for a location of the environment corresponding to a surface type from the map database 415.

The surface detection module 480 analyzes the visual-inertial data to determine characteristics of the surface type. In some embodiments, the surface detection module 480 compares the visual data to images of a plurality of floors with the same surface type and selects characteristics of a floor with an image that is most similar to the visual data. In other embodiments, the surface detection module 480 inputs the visual data and surface type to a machine learning model configured to identify characteristics for a surface type. The machine learning model may output a likelihood for each characteristic of a plurality of characteristics that the floor depicted in the visual data includes the characteristic. The surface detection module 480 may compare the likelihoods to a threshold likelihood and select characteristics with a likelihood higher than the threshold likelihood for the surface type at the location. The surface detection module 480 may store the characteristics in association with the surface type and location in the map of the environment.

The machine learning model may be trained on image data labeled with characteristics of a surface type associated with the image data. For example, the machine learning model may be trained on a plurality of images of different floorings, each labeled with a surface type and characteristics. For instance, a first set of image data may be labeled with the surface type “tile” and the characteristics “porcelain,” “plank pattern,” “glossy,” and “black.” A second set of image data may be labeled with the surface type “tile” and the characteristics “porcelain,” “plank pattern,” “wood grain,” “wood texture,” and “natural white.” Though both floorings in these sets of image data are of the surface type “tile,” the floorings have some characteristics that differ. The surface detection module 480 may store and train the machine learning model on sample image data of floorings received from a plurality of external flooring systems or external builders and contractors via an API presented at a client device 310.

The surface detection module 480 may also analyze the inertial data to augment the characteristics of the surface types of the environment. For instance, surface detection module 480 may determine, based on the inertial data, that the autonomous vacuum 100 moves more quickly over the surface type “tile” than the surface type “carpet.” The surface detection module 480 may build an understanding of how the autonomous vacuum 100 moves on different surface types and compare its understanding to the captured inertial data. For instance, the surface detection module 480 may determine that the autonomous vacuum typically moves at a speed of 10 miles per hour on rugs when a specific torque (or power) is applied to motors of the wheels. As the autonomous vacuum 100 traverses the environment, the surface detection module 480 may compare a real-time speed of the autonomous vacuum 100 as the specific torque is applied to the motors to recorded speeds for locations of the environment. The surface detection module 480 determines whether the speed at a location varies by more than a threshold percentage from a recorded speed, and if so, the surface detection module 480 determines that the surface type may have changed. In such instances, the surface detection module 480 may redetermine the surface type at the location and update the map in the map database 415. In another example, the surface detection module 480 may compare the torque required to make the autonomous vacuum 100 go a certain speed (e.g., 2 miles per hour) to the specific torque stored in relation to that location and speed for the autonomous vacuum 100. If the torque varies by more than a threshold percentage, the surface detection module 480 may redetermine the surface type of the location.

The surface detection module 480 also may determine a height of each surface type in the environment, e.g., by analyzing surface type or measuring distance from a ground plane to top canopy of surface. The surface detection module 480 may transmit (or send) an indication to the logic module 470 to lower the height of the cleaning head 140 to be resting on a surface at a location in the environment. The surface detection module 480 may determine, based on sensor data of the from the sensor system 118 via the logic module 470, a height of the wheels of the autonomous vacuum 100 and a height of the cleaning head 140. For example, the surface detection module 480 may determine the height of the wheels based on IMU data and the height of the cleaning head 140 based on visual data. The surface detection module 480 may store the height of the wheel and cleaning head 140 in relation to the location in the map database 415. In some embodiments, the surface detection module 480 may use the height of the cleaning head 140 and the wheels to determine boundaries between surface types in the environment. For example, on a dense (or thick) carpet, the cleaning head 140 may be at a height above the carpet while the wheels may be at a height below the carpet (due to the autonomous vacuum 100 sinking into the carpet). The cleaning head 140 may move up in height as the cleaning head 140 is moved over a rug on top of the carpet, while the wheels may move up the same amount in height as the wheels move over the rug. The surface detection module 480 may detect such change in height of the wheels and cleaning head 140 and store information corresponding to the location of the height change in the map database 415 as indicating a boundary between surface types.

The surface detection module 480 may store cleaning information in the map database 415 in association with each surface type. The cleaning information may include a particular fan speed, a particular noise level, a list of which components of the autonomous vacuum 100 should be used to clean the surface type, and the like. The surface detection module 480 may retrieve the cleaning information from external flooring systems associated with the surface type or external builders and contractors who entered the cleaning information via the API on a client device 310. For example, cleaning instructions for the surface type “carpet” may indicate to use an internal fan of the autonomous vacuum and cleaning information for the surface type “wood” may indicate to operate components of the autonomous vacuum 100 at a lower volume than for the surface type “rug.” The surface detection module 480 may communicate the cleaning information to the logic module 470 such that the logic module may use the cleaning information to determine how to control components of the autonomous vacuum 100 for cleaning.

In some embodiments, the surface detection module 480 receives real-time visual data of the environment and determines how to clean an area of the environment based on the surface type. In particular, the surface detection module 480 receives mess types detected in the section from the mess detection module 430 and/or task module 440. Alternatively, the surface detection module 480 may input the visual-inertial data of the environment along with the surface type to a machine learning model trained to detect messes of different mess types. The machine learning model may be trained on visual data of a plurality of surface types each labeled with the surface type and which pixels correspond to messes and of what mess types.

The machine learning model outputs a likelihood that the visual data includes a mess and likelihoods representing which mess type is shown. The surface detection module 480 compares the likelihood of including mess to a mess threshold, and if the threshold is exceeded, compares the likelihoods representing the mess types to a mess type threshold. The surface detection module 480 selects mess types associated with likelihoods over the mess type threshold. The surface detection module 480 accesses cleaning information for the determined surface type and mess type and sends the cleaning information to the logic module 470 to control the autonomous vacuum 100 to execute techniques based on the cleaning information. For instance, for a mess type of “spilled crumbs,” the cleaning information may indicate to vacuum over the mess and subsequently mop over the mess, which the flooring module may instruct the logic module 470 to control the autonomous vacuum 100 to do.

The surface detection module 480 may monitor the characteristics of the surface types in the environment as the autonomous vacuum 100 traverses the environment. For instance, the surface detection module 480 may use the machine learning model on image data captured in real-time of the environment and compare the characteristics determined by the machine-learning model to the characteristics stored from a corresponding location in the map database 415. If the surface detection module 480 detects a discrepancy in characteristics (e.g., new or missing characteristics), the surface detection module 480 may determine whether the autonomous vacuum 100 is traversing a boundary between surface types I the environment based on the height of the cleaning head and wheels and/or the visual data. The surface detection module 480 may redetermine the surface type for the location and update the map database 415 to indicate the surface type. The surface detection module 480 may send an indication of the boundary and/or redetermined surface type of the user interface module 490.

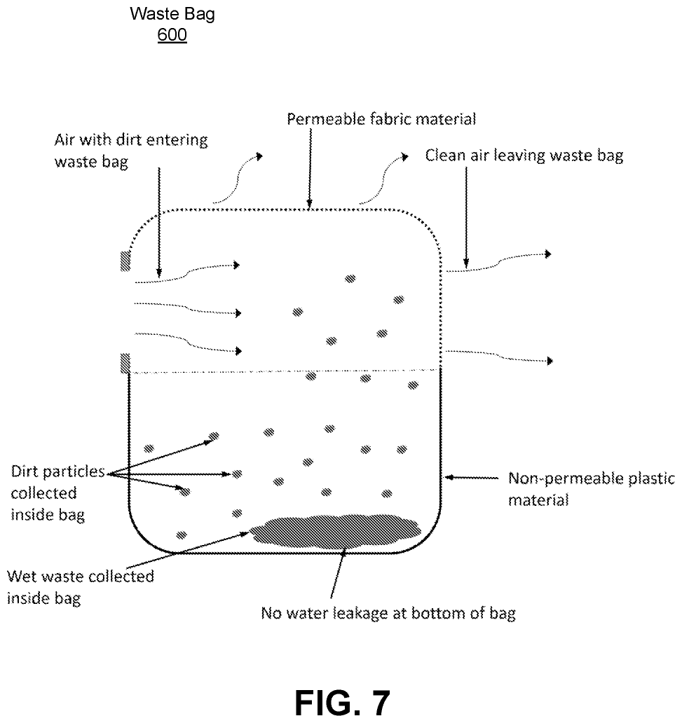

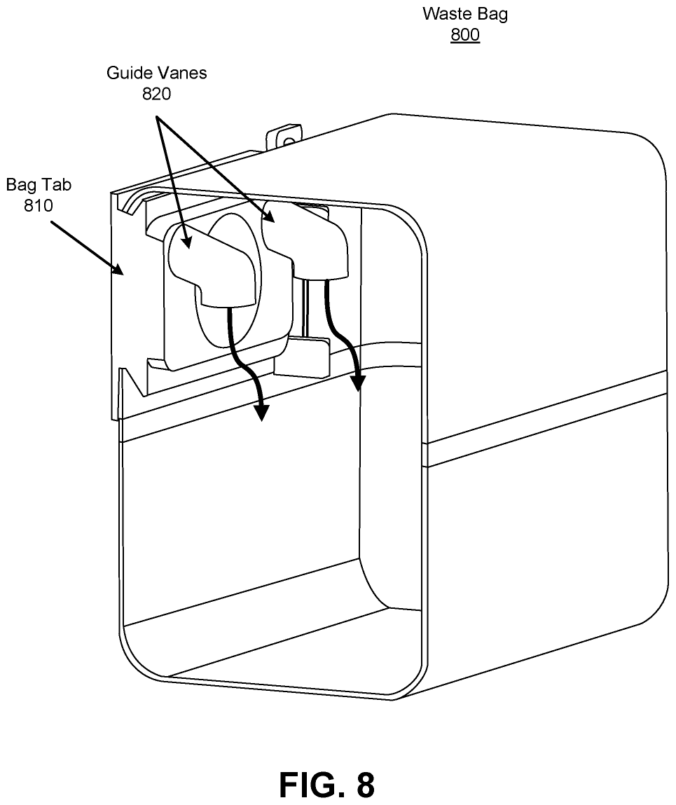

The user interface module 490 generates user interfaces for the client devices 320 and display 122. The user interfaces (or APIs) may include renderings of the environment, including meses, obstacles, and the like, retrieved from the map database 415. A rendering may be shown as a top-down view of the environment including representations of each surface type in corresponding areas of the environment. The user interfaces may also include a plurality of interactive elements that a user may interact with to control movement of the autonomous vacuum 100 and view sensor data captured by the autonomous vacuum 100. Examples of user interfaces are shown in FIGS. 7-8B. The user interface module 490 generated and transmits user interfaces in response to receiving indications from the client devices 320 and/or display 122 and updates the user interfaces based on interactions received from the client devices 320 and/or display 122.

The user interface module 490 may generate user interfaces that depict a surface type in a background. For instance, the user interface module 490 may receive a real-time location of the autonomous vacuum 100 from sensor system 118 via the logic module 470. The user interface module 490 may retrieve, from the map database 415, a surface type and characteristics of the location. The user interface module 490 may also receive real-time visual data off the surface type at or around the location from the sensor system 118 via the logic module 470. The user interface module 490 may insert a portion of image data corresponding to the surface type to a user interface as the background of the user interface. In some embodiments, the user interface module 490 may align the portion of image data to match an orientation of the pattern of the surface type in the real-time visual data. For instance, the background of the user interface may depict the surface type with a pattern in the same orientation as seen by a camera at the front 200 of the autonomous vacuum 100.

The user interface module 490 may layer interactive elements (or icons) on top of the background. In some embodiments, the interactive elements may remain stationary over the background of the user interface as the background renders a scroll animation, as described above. The interactive elements receive interactions from a user via the user interface that indicate to instruct the autonomous vacuum 100 and/or request data about the autonomous vacuum's 100 cleaning in the environment. In some embodiments, the user interface module 490 may alternately or additionally insert an element indicating the surface type to the user interface.

In some embodiments, the user interface module 490 may configure the image of the surface type in the background to slide within the background based on movement of the autonomous vacuum 100. For instance, the user interface module 490 may determine a real-time speed of the autonomous vacuum from IMU, motor, and/or visual data captured by the sensor system 118 and sent to the user interface module 490 via the logic module 470. The user interface module 490 may configure the image data of the background to be animated at a speed that is linearly related to the real-time speed. The user interface module 490 may further configure the image data in the background to mirror an alignment of flooring in the environment based on real-time image data of the environment. For example, the user interface module may align grout lines from the image data in the background with grout lines in the real-time image data, such that the patterns shown in the background and the real-time image data match. In another example, the user interface module 490 may determine an orientation of fibers in a carpet and align image data of the carpet in the background of the user interface to match real-time image data of the carpet captured at the front 200 of the autonomous vacuum 100.

In some embodiments, the user interface module 490 includes one or more interactive elements for users, such as the user of the autonomous vacuum 100 or external builders or operators, to enter characteristics and cleaning information for surface types in an environment. For instance, the user interface module 490 may generate a user interface with a rendering of the environment with the floor in the rendering divided into selectable sections. Each selectable section may correspond to a surface type determined for an area of the environment. The user interface module 490 may receive a selection of a selectable section and insert information about the surface type and characteristics of the area with the surface type in the user interface. The user interface module 490 may also add one or more text boxes or selectable lists to the user interface that a use may interact with to add more characteristics and cleaning information to the map database 415 for the surface type and section. For instance, a contractor may change a surface type and characteristics of the section after replacing flooring in the environment.

In some embodiments, the user interface module 490 may communicate with a camera of the client device 310 presenting the user interface to receive machine-readable codes. The user interface module 490 may communicate the machine-readable codes to external systems (e.g., the Internet) to collet information associated with the machine-readable codes (e.g., characteristics and cleaning information. For example, a contractor, after renovating the environment by replacing carpet with tile, selects an area of the environment in a rendering presented the user interface. The user interface module 490 receives the selection and edits the user interface to include a scan icon that communicates with a camera of a client device 310 presenting the user interface. The contractor may scan, using the scan icon, a machine-readable code associated with the tile. The user interface module 490 receives the machine-readable code and retrieves information about the tile from a website associated with the machine-readable code. The user interface module 490 updates the map of the environment to include the surface type (e.g., “tile”) and characteristics and cleaning information from the retrieved information.

In some embodiments, the user interface module 490 may generate a user interface including a joystick element that is configured to receive interactions corresponding to desired movements of the autonomous vacuum 100. For example, the user interface module 490 may receive an interaction from the joystick element indicating movement to the left and communicate with the logic module 470 to move the autonomous vacuum 100 to the left tin the environment. The user interface module 490 may compare movements corresponding to interactions with the joystick button to the map of the environment based on a real-time location of the autonomous vacuum 100. If a movement would lead to an error for the autonomous vacuum 100 (e.g., surpassing a virtual border, hitting a wall, running the mop roller on carpet, etc.), the user interface module 490 may send an alert of the error via the user interface rather than proceeding to instruct the logic module 470 based on the movement.

The user interface may also include one or more interactive elements for manually controlling other components of the autonomous vacuum, such as the cleaning rollers, solvent motor 120, vacuum pump 115, actuator assembly 138, and sensors of the sensor system 118. For example, the user interface module 490 may receive an interaction with a mop roller button via the user interface and send an indication to the logic module 470 to activate a mop roller of the autonomous vacuum 100. The user interface may also include interactive elements for controlling a noise level emitted by the autonomous vacuum 100 during cleaning. For instance, the user interface module 490 may include an interactive slider configured to raise or lower the noise emitted from the autonomous vacuum 100 during operation as the interactive slider is slid to one direction or the other, respectively. The user interface module 490 may communicate with the logic module 470 to control the noise of components of the autonomous vacuum 100 and may send notifications via the user interface regarding change sin noise level.

### Waterproof Waste Bag

The waste bag implemented in the autonomous vacuum is configured to collect debris vacuumed by the autonomous vacuum. The waste bag may be semi-waterproof or waterproof to collect both dry waste and liquid waste. In one or more embodiments, the waste bag is removably coupled to a bin cavity of the autonomous vacuum.

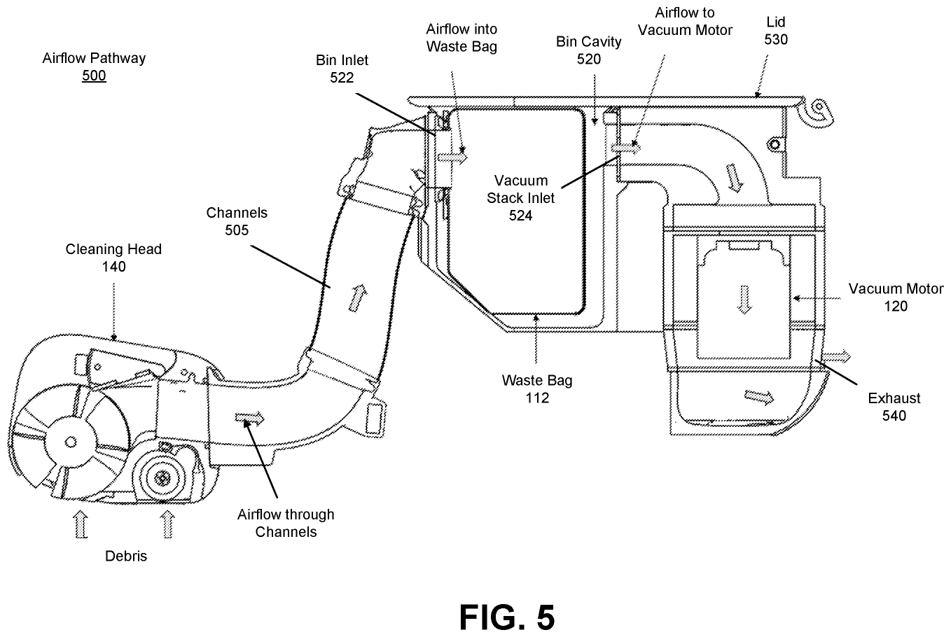

FIG. 5 is a cross-sectional view of an airflow pathway 500 for the autonomous vacuum, according to one or more embodiments. The airflow pathway 500 is illustrated with the grey arrows flowing into the cleaning head 140, through the channels 505 (e.g., the dry channel 132 and/or the liquid channel(s) 134 of the connection assembly 130), into the waste bag 112 situated in a bin cavity 520 via a bin inlet 522, through a vacuum stack inlet 524 to the vacuum motor 120, and out of the autonomous vacuum through an exhaust 540.

The waste bag 112 is connected in the airflow pathway 500. Being situated in the bin cavity 520, the waste bag 112 can operate as a filter allowing debris to be collected into the waste bag 112 whilst filtered air passes through an air-permeable membrane of the waste bag 112. The pores of the permeable membrane determine the filter efficiency (typically rated in HEPA) and airflow characteristics. The smaller the pore size, the higher the filter efficiency and lower the airflow for the same surface area.

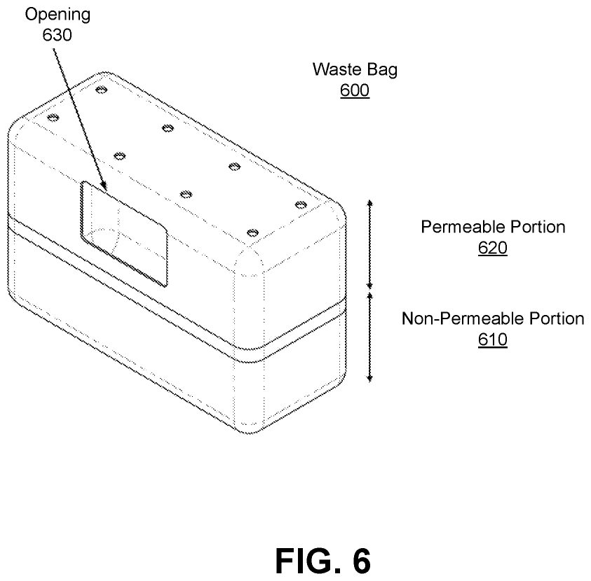

FIG. 6 is a perspective view of a semi-waterproof waste bag 600 for the autonomous vacuum, according to one or more embodiments. The waste bag 600 is an embodiment of the waste bag 112. In general, the waste bag 600 includes a non-permeable portion 610, a permeable portion 620, and an opening 630. Other components that may be implemented in the waste bag are further described in subsequent figures.

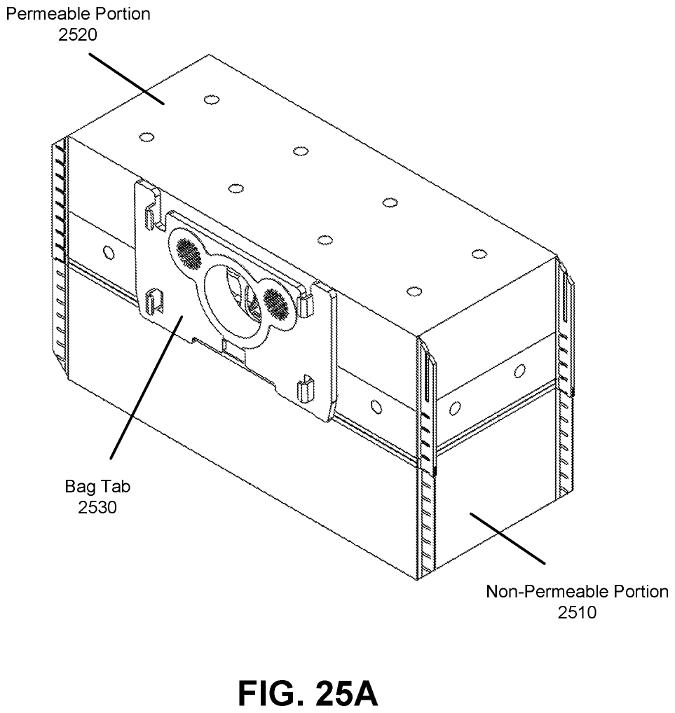

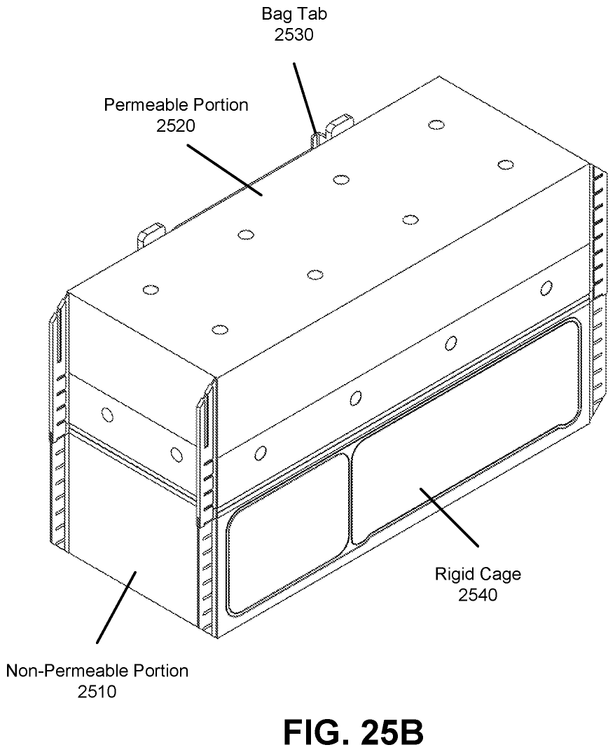

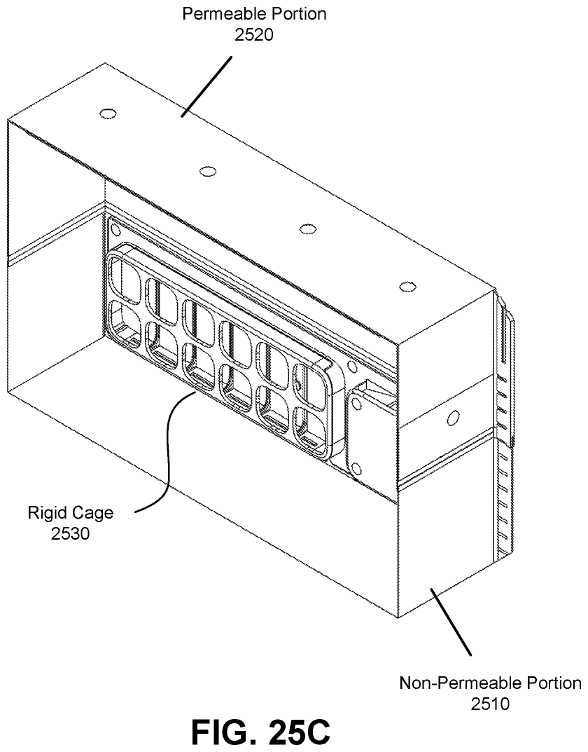

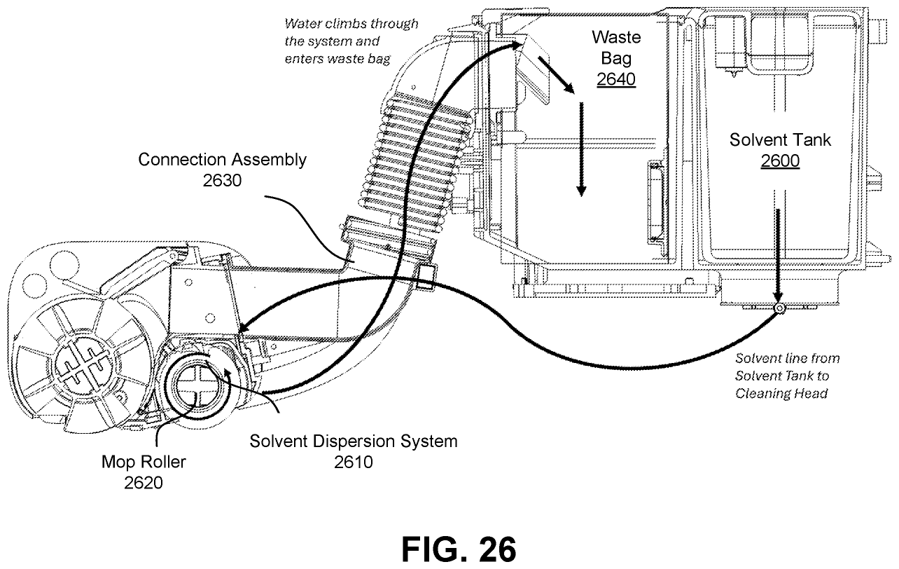

The non-permeable portion 610 collects the waste without causing any leakage outside the bag. The non-permeable portion 610 may be formed of a material that is non-permeable to liquid (e.g., water) or air. The non-permeable portion 610 may be formed from a plastic, a waterproof fabric, a latex or another rubber-based material, metal, or some other waterproof material. In some embodiments, the non-permeable portion 610 may be composed of multiple layers including at least one waterproofing layer. For example, an otherwise non-waterproof fabric can be coated with a waterproofing material. The non-permeable portion 610 may be transparent allowing for at least two advantages: aiding in accurate sensing of the amount of dirt collected (e.g., with a light-based sensing system further described in FIGS. 25, 26A, and 26B); and aiding the user in visual inspection of waste fullness in the bag.

The permeable portion 620 permits airflow and creates a filter barrier for filtering the air suctioned from the cleaning head. The permeable portion 620 may be formed of a material that is permeable to liquid and/or air. Example materials include fabrics, other types of membrane, etc. The permeable portion 620 is connected to the non-permeable portion 610 with, e.g., ultrasonic weld, staples, adhesive, stitching, etc.

FIG. 7 is a cross-sectional view of the semi-waterproof waste bag 600 for the autonomous vacuum, according to one or more embodiments. The division of permeable and non-permeable sections determine the amount of debris that can be collected inside the waste bag 600. The amount may be characterized by a fill line at the interface between the non-permeable portion 610 and the permeable portion 620. To prevent leakage, the autonomous vacuum may use sensors to ensure that the waste bag 600 is replaced when the debris level comes close to or meets the fill line. In one embodiment, the fill line is set at 50% of the non-permeable portion 610 and 50% of the permeable portion 620. This ratio maximizes the usage of the waste bag 600. A small permeable portion 620 increases the tendency of dirt particles to clog the fabric pores quickly, which can decrease the vacuum suction power. On the other side of the tradeoff, a small non-permeable portion 610 limits the amount of liquid waste collection. The ratio is balances the two constraints.

FIG. 8 is a cutaway perspective view of the semi-waterproof waste bag 800 and a bag tab 810, according to one or more embodiments. The waste bag 800 is an embodiment of the waste bag 600. In one or more embodiments, the semi-waterproof waste bag 800 includes a bag tab 810 and one or more guide vanes 820. The bag tab 810 may be a rigid structure for coupling to the bin cavity. The bag tab 810 may be formed of rigid, inelastic materials, e.g., plastic, metal, etc. The bag tab 810 may be 3D-printed, injection-molded, or manufactured through some other 3D structure forming process. The bag tab 810 may be substantially planar in shape, and may include features for coupling to the bin cavity.

Leakage may also occur through the permeable portion, caused by high velocity water droplets hitting the back of the waste bag's permeable portion. To address this problem, the waste bag 800 may include the guide vanes 820 that guide entry of the liquid waste into the bottom of the waste bag 800. The guide vanes 820 may be open-ended channels that extend downward from a coupling point to the bag tab 810. As shown in FIG. 8, the guide vanes 820 may include an angled portion connected to a vertical portion. The angled portion is angled downwards from a top portion of the bag tab 810. The vertical portion directs the liquid waste downwards towards a bottom of the waste bag 800. Due to the internal walls and angle of guide vanes 820, high velocity water particles hit the wall and are redirected towards the bottom of the waste bag 800. This mitigates water particles hitting the permeable portion of the waste bag 800 and thus avoiding any leakage. The guide vanes 820 may be formed with the bag tab 810, i.e., monolithic, or may be formed separately and then attached.

In one or more embodiments (not illustrated), the waste bag may be formed of a waterproof material. The material can be permeable to air and non-permeable to water. For example, the material can leverage surface tension to create the selective permeability. In general, the surface tension of air is low, whereas the surface tension of liquids is high. The material can target such disparity to achieve selective permeability. In one example, the material is porous in nature, permitting air permeability (e.g., under vacuum pressure); however, liquid waste (e.g., solvent, dirt, etc.) would not be permeable, thereby remaining contained within the waste bag.

### Bag-Bin Seal

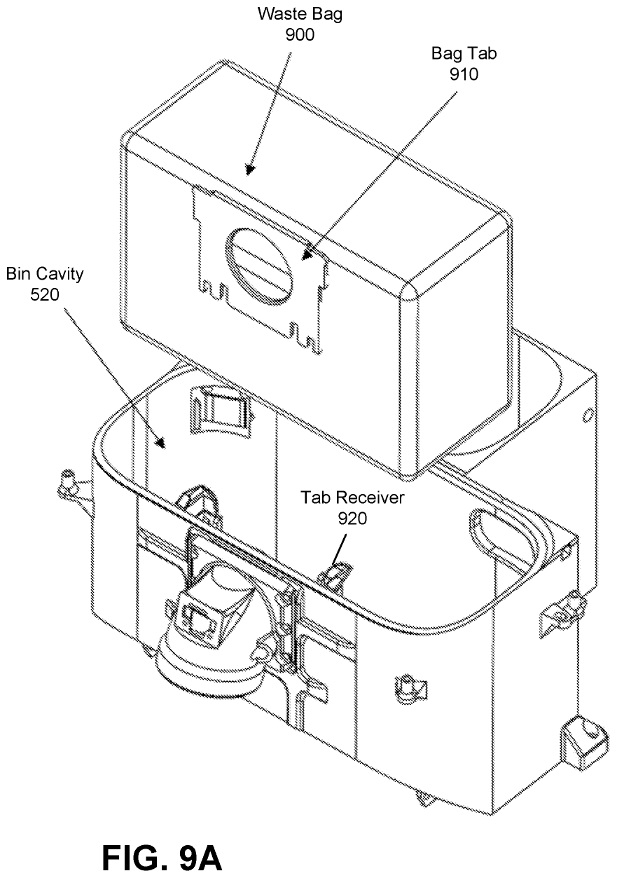

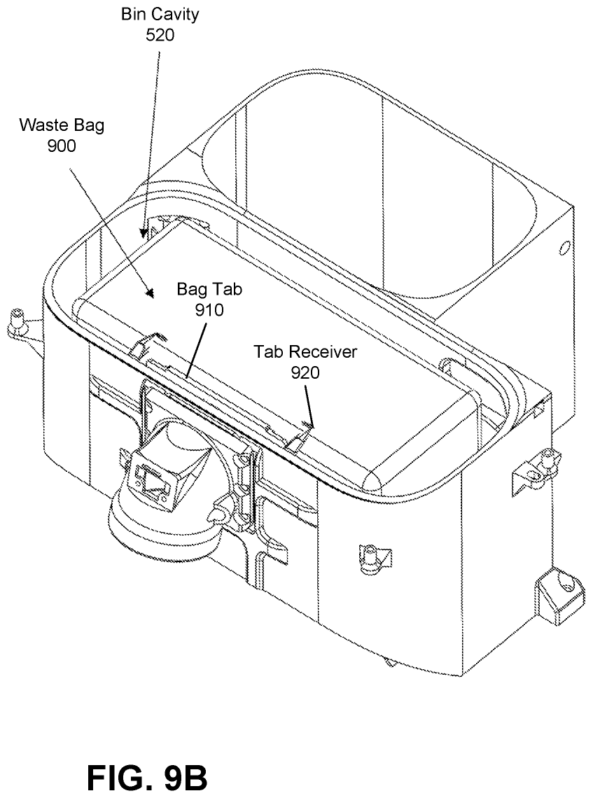

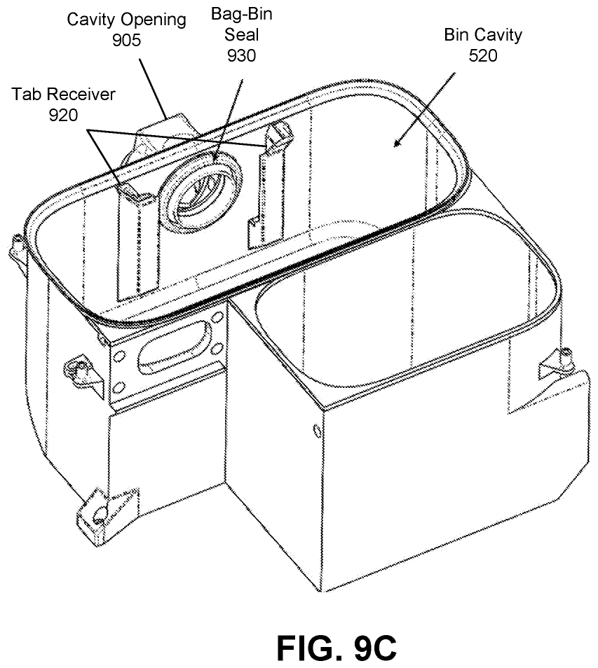

FIG. 9A is a perspective view of the semi-waterproof waste bag 900 removed from a bin cavity of the autonomous vacuum, according to one or more embodiments. The waste bag 900 is an embodiment of the waste bag 600, including a bag tab 910. The bag tab 910 slots into a tab receiver 920 of the bin cavity 520. The bag tab 910 includes a hole that forms the opening into the waste bag 900. The bag tab 910 includes one or more structural features for keying into the tab receiver 920, which includes one or more complementary structural features for coupling to the structural features on the bag tab 910.

FIG. 9B is a perspective view of the semi-waterproof waste bag 900 installed into the bin cavity 520 of the autonomous vacuum, according to one or more embodiments. As noted, the bag tab 910 slots into the tab receiver 920. As shown in FIG. 9B, a top of the waste bag 900 may be flush with a top of the bin cavity 520.

FIG. 9C is a perspective view of the bin cavity 520 including a bag-bin seal 930 implemented around a cavity opening 905, according to one or more embodiments. The bag-bin seal 930 is situated around the tab receiver 920 in the bin cavity 520.

The bag-bin seal 930 contacts the bag tab of the waste bag (e.g., the bag tab 910 of FIGS. 9A & 9B) to form a seal. The bag-bin seal 930 is formed of a flexible, compliant material, e.g., an elastomer, a rubber, or some other like material. The bag-bin seal 930 achieves a low-closure force seal while ensuring no leakage during operation.

The tab receiver 920 is configured to receive the bag tab of the waste bag (e.g., the bag tab 910 of FIGS. 9A & 9B). In one or more embodiments, the tab receiver 920 includes two different receiving elements positioned apart from one another matching the width of the bag tab. In one or more embodiments, the receiving elements center around the cavity opening 905 and are positioned such that the cavity opening 905 aligns with the bag opening. In one or more embodiments, a top portion of the receiving elements are flared, so as to guide the bag tab of the waste bag into the tab receiver 920.

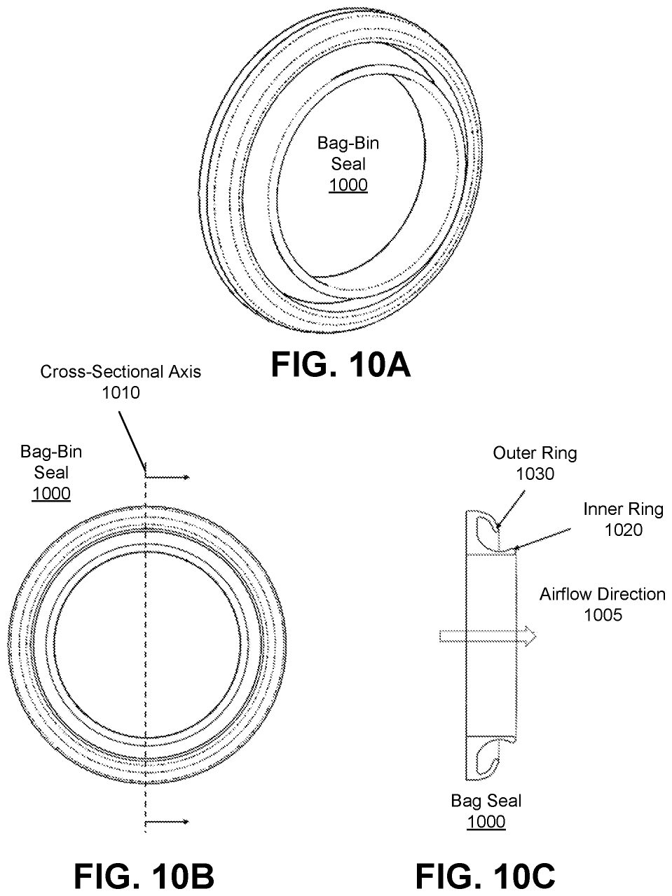

FIG. 10A is a perspective view of a bag-bin seal 1000, according to one or more embodiments. FIG. 10B is a planar view of the bag-bin seal 1000, according to one or more embodiments. FIG. 10C is a cross-sectional view of the bag-bin seal 1000, according to one or more embodiments. The cross-sectional view of the bag-bin seal 1000 is taken along the cross-sectional axis 1010 shown in FIG. 10B.

In one or more embodiments, the bag-bin seal 1000 is of annular shape. In other embodiments, the bag-bin seal 1000 may be of a differing shape that matches to the cavity opening and/or the waste bag opening. The bag-bin seal 1000 may be formed of a compliant material. The bag-bin seal 1000 includes an inner ring 1020 and an outer ring 1030, which aid in forming the seal against the bag tab. The inner ring 1020 and outer ring 1030 protrude from the base plane (i.e., the left-side plane in FIG. 10C). Each is connected to a planar portion of the bag-bin seal 1000 at their respective base, with an end of the protrusion defined as a tip.

The inner ring 1020 guides water (and any other liquid waste into the waste bag). The inner ring 1020 is substantially perpendicular to the base plane. The inner ring 1020 has varying thickness from its base to tip. The base is of greater thickness, a middle portion is of minimal thickness, and the tip is of thickness greater than the middle portion and less than the base.

This inner ring 1020 acts as the primary mode of water and liquid waste transfer to the waste bag. The water particles, being heavier, tend to accumulate on the bottom wall of the inner ring 1020. The inner ring 1020 provides a path for water to drop directly into the waste bag. See The inner ring 1020 extends inside the waste bag (when installed) and this allows for water to directly flow into the waste bag. Without the inner ring 1020, liquid waste can get collected in the cavity between outer ring 1030 and the bag tab. And, upon user removal of the waste bag, liquid waste can stay inside the bin cavity. The inner ring 1020 tackles this issue to mitigate liquid waste collection in the bin cavity.

The inner ring 1020 may have a thin middle portion, to permit low force bending of the inner ring 1020 during installation of the waste bag. Since the bag tab is designed to engage past the seal in vertical direction, this compliant section would bend and ensures a low force of engagement.

The outer ring 1030 provides the primary seal between the waste bag and the bin cavity. The outer ring 1030 has an arcuate shape, and bends towards a center axis of the bag-bin seal 1000. The outer ring 1030 is of a shorter length than the inner ring 1020, i.e., the inner ring 1020 extends further than the outer ring 1030. The outer ring 1030 may be of uniform thickness from its base to tip.

This outer ring 1030 acts as the primary seal for air and or fluid. As such, dry waste like household dust, hair and lighter dirt particles do not enter the bin cavity from the vacuum airflow pathway. The aim of the seal is to prevent any leakage from the waste bag to the bin cavity from the sealing interface. During operation, the region around the seal is at lower pressure than the region inside the bag. So, sealing prevents weakened vacuum suction power.

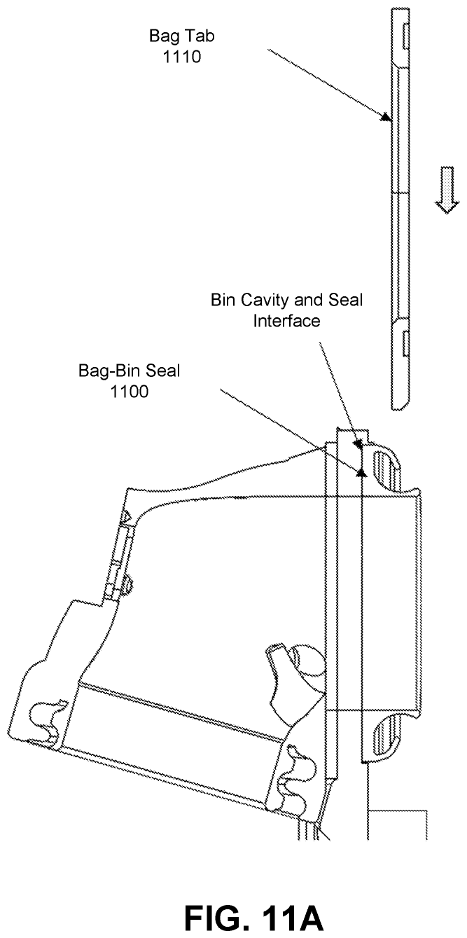

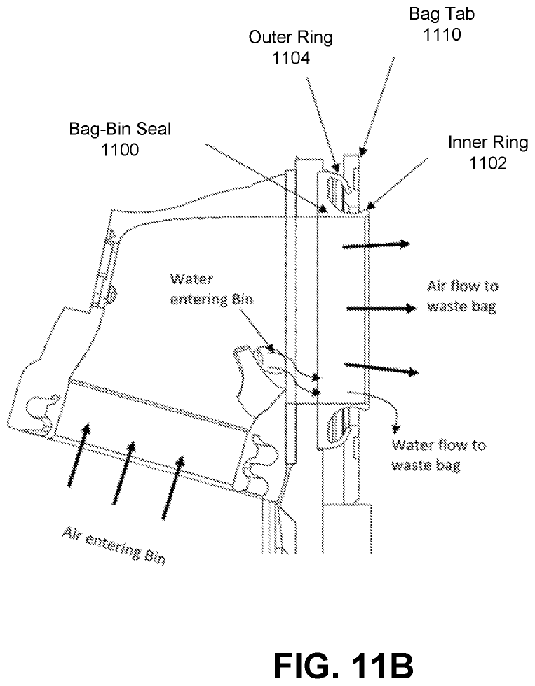

FIG. 11A is a cross-sectional view of the bag tab 1110 of the semi-waterproof waste bag interacting with the bag-bin seal 1100 of the bin cavity, according to one or more embodiments. FIG. 11B is a cross-sectional view of the bag tab 1110 and the bag-bin seal 1100 when the waste bag is installed into the bin cavity, according to one or more embodiments. The horizontal (on the page) positioning of the bag tab 1110 relative to the bag-bin seal 1100 is determined based on the tab receiver. When installed, the outer ring 1104 of the bag-bin seal 1100 forms a seal with the bag tab 1110. The inner ring 1102 provides the channel to direct liquid waste into the waste bag.

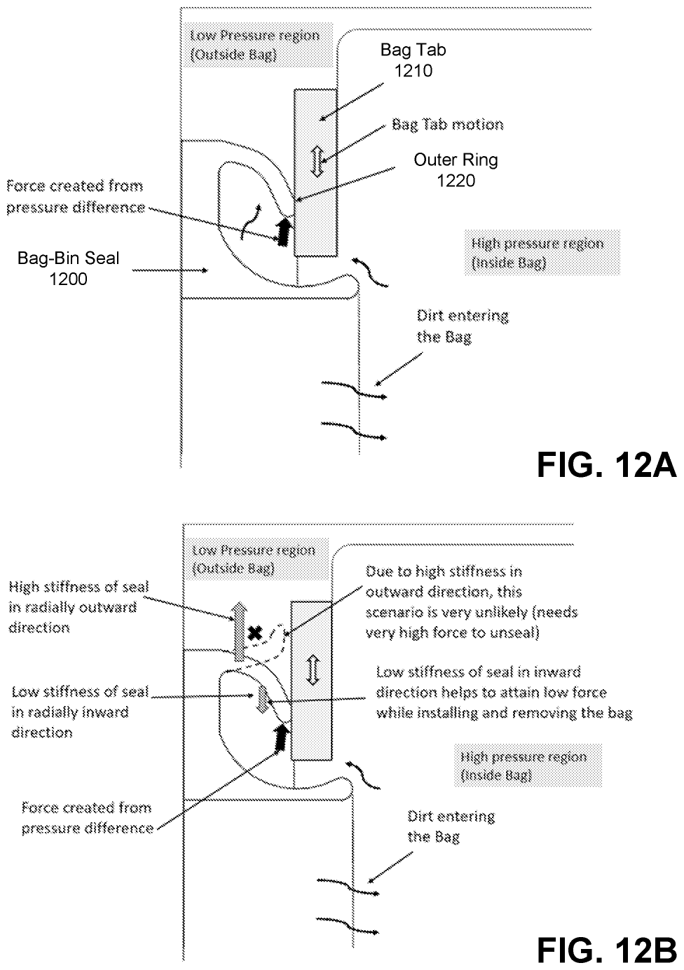

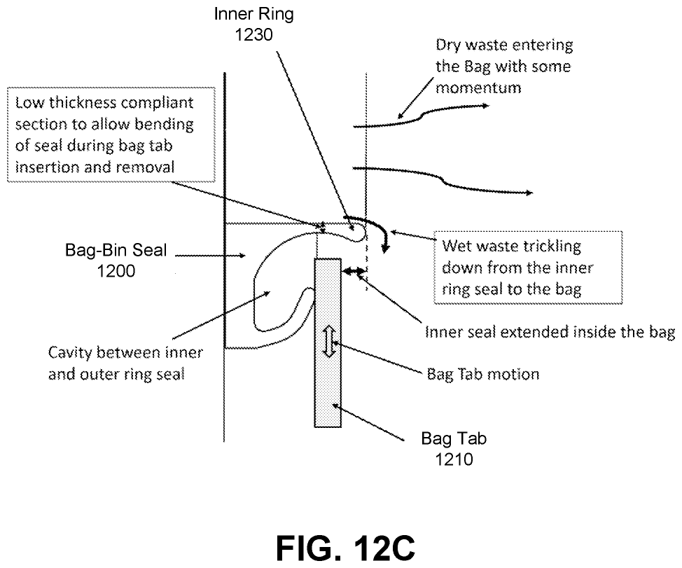

FIG. 12A is a first close-up cross-sectional view of a top portion of the bag tab 1210 and the bag-bin seal 1200, according to one or more embodiments. FIG. 12B is a second close-up cross-sectional view of the top portion of the bag tab 1210 and the bag-bin seal 1200, according to one or more embodiments. FIG. 12C is a close-up cross-sectional view of a bottom portion of the bag tab 1210 and the bag-bin seal 1200, according to one or more embodiments.

Since the aim is to provide a robust sealing with a low closure force, the outer ring 1220 has a cantilevered based design, in one or more embodiments. Due to the angled profile towards the direction of high pressure, the outer ring 1220 offers an advantage on gaining high stiffness in one direction vs the other. It is required for the seal to have high stiffness in the direction of force to avoid deflection and in turn leakage. However high stiffness of the outer ring 1220 also causes high force of engagement. Using the angled profile as shown in FIG. 12B solves this issue by keeping high stiffness in only the direction needed and lowering stiffness in other direction. This enables a low closure force seal which creates a better user experience since the removal and addition of bag would not require high force. The angle and the geometry of the seal is optimized to keep the ratio of engagement force to sealing force as low as possible.

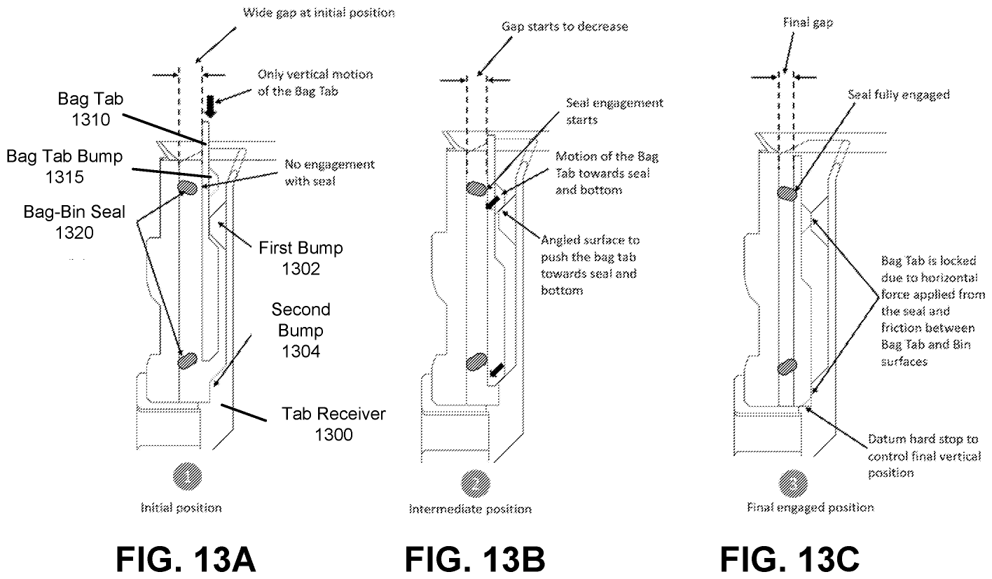

At the engaged position, the outer ring 1220 deforms elastically and fills in the surface irregularities on the bag tab 1210's surface. Due to outer ring 1220's flexible nature and its deformation, it adds a barrier for air and any dust particles and hence stops any leakage. The deformation of the outer ring 1220 is achieved by applying a force from the bag tab 1210 in the horizontal direction. The locking and position of bag tab is explained further in dual bump locking mechanism illustrated in FIGS. 13A-C.

FIG. 13A is a first close-up cross-sectional view of the bag tab 1310 and the bag-bin seal 1300, according to one or more embodiments. FIG. 13B is a second close-up cross-sectional view of the bag tab 1310 and the bag-bin seal 1300, according to one or more embodiments. FIG. 13C is a third close-up cross-sectional view of the bag tab 1310 and the bag-bin seal 1300, according to one or more embodiments. FIGS. 13A-C illustrate a dual-bump locking mechanism for a compression fit of the bag tab 1310 to the tab receiver 1300.

The tab receiver 1300 includes a plurality of bumps. In the embodiments shown, the tab receiver 1302 includes a first bump 1302 and a second bump 1304. The bag tab 1310, itself, contains a bag tab bump 1315, that interacts with the bumps of the tab receiver 1300.

The engagement of seal is executed through a mechanical locking mechanism. For any elastomeric seal to function properly, the bag-bin seal 1320 needs to deform and fill in the gap created from surface irregularities on the bag tab 1310. This needs an external force to be applied on the bag-bin seal 1320, i.e., a sealing force. In this embodiment, the sealing force is applied through the bag tab 1310's installed position.

In the initial position shown in FIG. 13A, the bumps of the tab receiver 1300 are not engaged. In this position the bag tab 1310 can travel freely in the vertical direction. At this position there is no force from the seal and the user will also not experience any resistance during the initial position.

In the intermediate position shown in FIG. 13B, the bumps of the tab receiver 1300, having an angled surface, are initiated by vertical translation of the bag tab 1310. Due to the angled surfaces, now the bag tab 1310 is pushed towards the bag-bin seal 1320. The first bump 1302 interacts with the bag tab bump 1315, whereas a bottom of the bag tab 1310 interacts with the second bump 1304. In this state, the user starts experiencing some resistance since the deformation of bag-bin seal 1320 is creating a counteracting force. However, the angle of the bumps of the tab receiver 1300 are designed to gradually increase the sealing force, i.e., without any abrupt change in force. The friction between the angled surfaces of the bumps of the tab receiver 1300 and the counterpart surfaces of the bag tab 1310 also affect the sealing force (i.e., experienced by the user during installation). A very smooth finish and low friction materials are chosen to minimize the friction force.

In the final engaged position shown in FIG. 13C, the optimal interference of the bag-bin seal 1320 with the bag tab 1310 creates a robust seal. The design also has a hard stop in vertical direction which controls the final position of the Bag Tab. The force generated from deformation of elastomer seal pushes the bag tab 1310 in the horizontal direction and the friction between the bag tab 1310 and the bumps of the tab receiver 1300 lock the bag tab 1310 in the fully-engaged position.

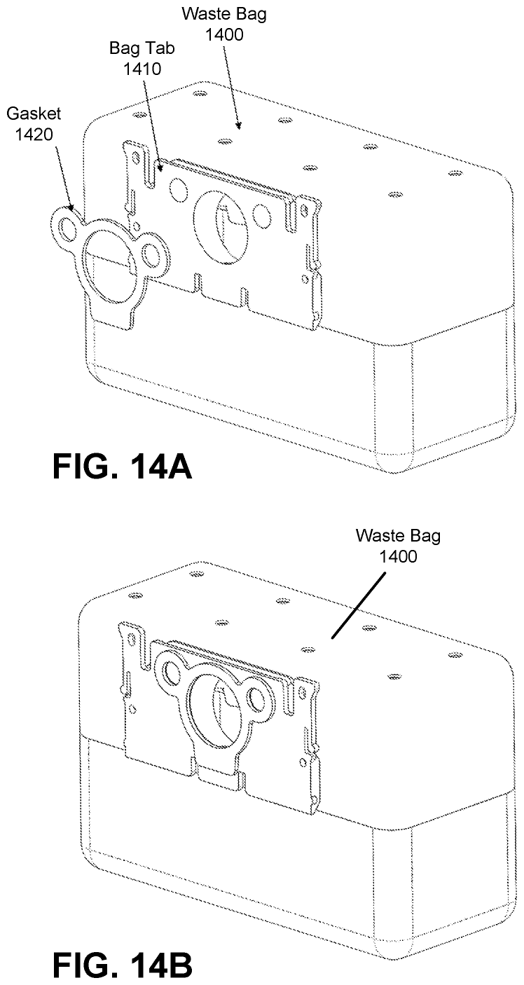

FIG. 14A is an exploded view of the waste bag 1400 with a gasket 1420 coupled to the bag tab 1410, according to one or more embodiments. FIG. 14B is a constructed view of the waste bag 1400 with the gasket 1420 coupled to the bag tab 1410, according to one or more embodiments.

The sealing action can be achieved using the gasket 1420. The gasket 1420 provides the seal by deforming to the shape of the sealing surface's irregularities through compression. In this solution, the gasket 1420 is attached to the bag tab 1410. When the bag tab 1410 is engaged with the tab receiver, the gasket 1420 compresses to form a seal.

The compression of the gasket 1420 is pre-determined and is a factor of sealing force requirements. The gasket-based seal provides an advantage due to availability of a wide variety of materials, that can be easily cut to shape as per the design requirements. In this case the thickness and the width of the gasket is chosen and optimized to obtain robust sealing with low sealing force while accounting for tolerances in sealing parts.

The compression of the gasket at final state can be achieved by using dual-bump locking mechanism as described above in FIGS. 13A-C.

As shown in FIGS. 14A & 14B, the gasket 1420 is substantially planar in shape and includes one or more holes that align to holes in the waste bag 1400. In the embodiment shown, the waste bag 1400 includes three holes, such that the gasket 1420 also includes three holes. In other embodiments, the waste bag 1400 has a differing number of holes, and the gasket 1420 is shaped and sized to match the differing design.

### Mitigation of Bin Cavity Condensation

The waste bag generally collects dry and/or liquid waste. The liquid waste collected inside the waste bag is typically a mixture of water and contaminants. Due to conditions inside the bin cavity, the water content gains enough thermal energy to undergo the process of evaporation. This phenomenon happens at standard room temperature and humidity conditions, but can be accelerated with high temperature created from operation of the autonomous vacuum.

When water content undergoes evaporation, it leaves the waste bag through its permeable surfaces. The evaporated vapor eventually condenses outside the bag on various surfaces of the bin cavity. This gives rise to two critical issues. (1) When the vacuum motor is turned on and there is water in the bin cavity, the water is pulled into the motor and causes damage to the electronics and elements of the vacuum motor. The damage can result in instant failure of electronics or long-term degradation of motor elements depending on the amount of moisture content. (2) If a user opens the lid at this state, they would find water all around the bin cavity and the lid. To remedy the condensation, the user would need to wipe up the condensation-covered surfaces of the bin cavity. Four solutions are described below. Embodiments may combine any number of the solutions.

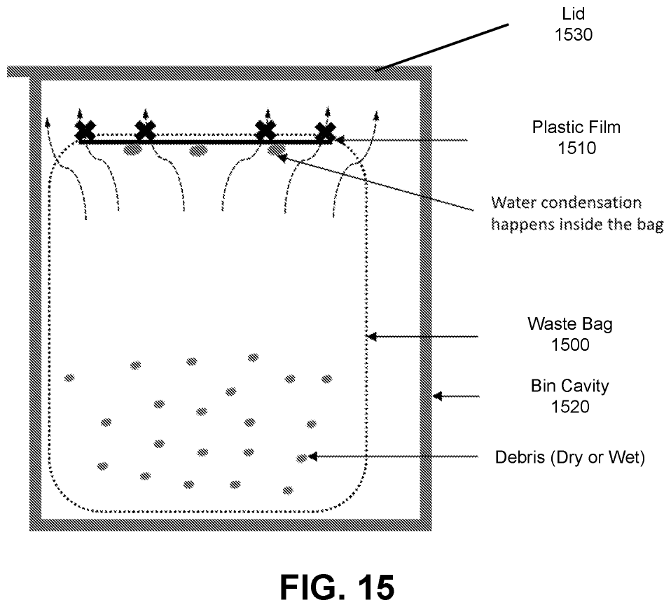

FIG. 15 is a cross-sectional view of the waste bag 1500 with a plastic film 1510 for mitigation of condensation in the bin cavity 1520, according to one or more embodiments. This solution describes the modification of the waste bag to keep the moisture content inside the bag as much as possible by reducing the amount of water vapor leaving the bag.

The plastic film 1510 is added to the top of the waste bag 1500. The plastic film 1510 is non-permeable, such that water vapor from the liquid waste at the bottom of the waste bag 1500 condenses on the plastic film 1510, thereby significantly reducing the amount of moisture leaving the waste bag 1500. Since evaporation is still happening at the same rate, this solution will not reduce the rate of evaporation but it does add a barrier for the water to be condensed inside the waste bag 1500 rather than escaping the waste bag 1500.

The dimensionality of the plastic film 1510 and the joining method may be optimized to ensure the maximum amount of moisture is retained while maintaining as much surface area of the permeable portion. A joining method of ultrasonic welding may be used. The method would use spot welding in discrete locations. Using spot welding, as compared to a typical line weld, enables to use the plastic sheet to add a moisture barrier inside the bag with minimal impact on surface area of the permeable portion. A continuous line weld would essentially seal the entire top surface fabric and reduce the fabric surface area for dirt collection. Spot weld eliminates that issue by only joining at discrete locations and not blocking the path of airflow while still blocking the path for water vapor. Other methods of joining include: staples, adhesive, stitching, etc.

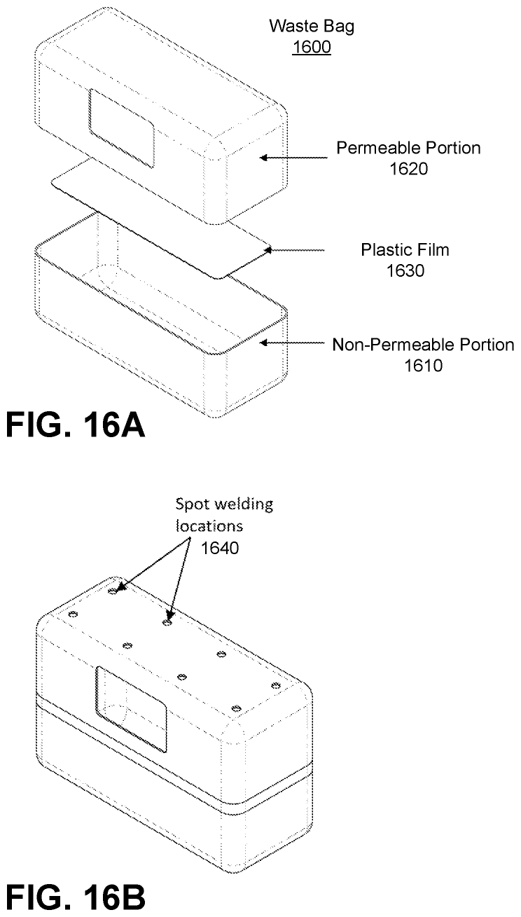

FIG. 16A is an exploded view of the waste bag 1600 with the plastic film 1630, according to one or more embodiments. FIG. 16B is a constructed view of the waste bag 1600 with the plastic film showing spot welding locations 1640, according to one or more embodiments.

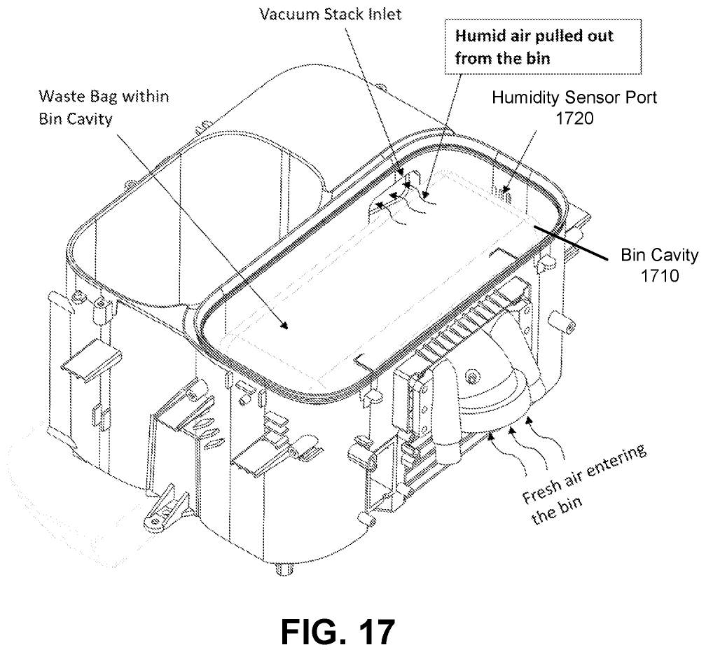

FIG. 17 is a perspective view of the autonomous vacuum performing vacuum removal of humid air in the bin cavity, according to one or more embodiments. This is a second solution to bin cavity condensation. This solution utilizes the on-board vacuum motor which is primarily used to generate airflow for dirt collection from the ground. The vacuum motor is run at a slow speed (e.g., low revolutions per minute) to remove the humid air from the bin cavity 1710 before it condenses. This may be referred to as Ventilation Mode in the system. The speed of vacuum motor is optimized in a way that it creates just enough circulation of air to remove humid air from the bin, while operating quietly. During ventilation mode, fresh air mixes with the humid air inside the bin and brings down humidity to a lower level. In some embodiments, the bin cavity 1710 includes a humidity sensor, e.g., positioned at the humidity sensor port 1720.

This solution can be implemented in a variety of logics. For example, it may be configured for (1) Continuous ventilation by running vacuum motor at low rpm when the autonomous vacuum is idle. It may be configured for (2) Continuous ventilation by running vacuum motor at low rpm but only after a mopping session. Since there is only addition of water during mopping cycle, this logic is to use that information to run the motor only when the water is present in the bag. The cycle can be reset whenever a new bag is added; Further, it may be configured to have (3) Time based ON-OFF cycle with pre-determined frequency and run time. This can be run either only after Mopping or at anytime the cleaning robot is idle. This can be timed during long periods of inoperation, e.g., nighttime, or directly after a planned mopping cycle. In addition, it may be configured to (4) Actively sense the humidity inside the bin cavity and running the ventilation mode only when the humidity level reaches a certain threshold. An additional sensing element measures humidity in the bin cavity. This provides the most accurate control on the run time of the ventilation mode and would prevent running the motor unnecessarily. FIG. 17 illustrates one example humidity sensor port. Once the humidity reaches a certain level, the motor operates in the ventilation mode until the humidity level is lowered below the threshold. The cycle is repeated once the humidity level reaches the threshold again. The threshold is also optimized in regards to reducing the frequency of cycles (higher threshold RH %) while still ensuring the reliability of vacuum motor is not compromised. The threshold would also ensure there is no water condensation within the bin. This helps in increasing the reliability of the motor and also avoid unnecessary power consumption.

Further, it may be configured to (5) Actively sense the environmental conditions to further tune and optimize the operating parameters for the ventilation mode. The sensing for this method would need to: sense the humidity inside the bin, sense/estimate the temperature of the lid and bin internals, sense/estimate the temperature of the environment (outside robot). This would allow the cleaning robot to adjust the operating parameters depending on the user's home and its external conditions. Since water condensation is a function of both humidity and temperature levels, this implementation would take those parameters to model it correctly for any environment the robot encounters. The implementation is to estimate the dew point temperature and use it to drive the ventilation mode. Using the humidity and temperature of the environment, a dew point temperature can be calculated. This is the surface temperature where the condensation actually begins. This information can be used to have an adjustable threshold instead of a fixed humidity level threshold. As an example, the advantage of this method would be that during summers, the frequency of ventilation mode can be lower. This stems from the fact that the external temperature is typically higher during summers, and the dew point is also higher for same humidity level. Leveraging these environmental conditions, a higher humidity level threshold can be prescribed for Ventilation Mode while still ensuring there is no condensation in the bin cavity.

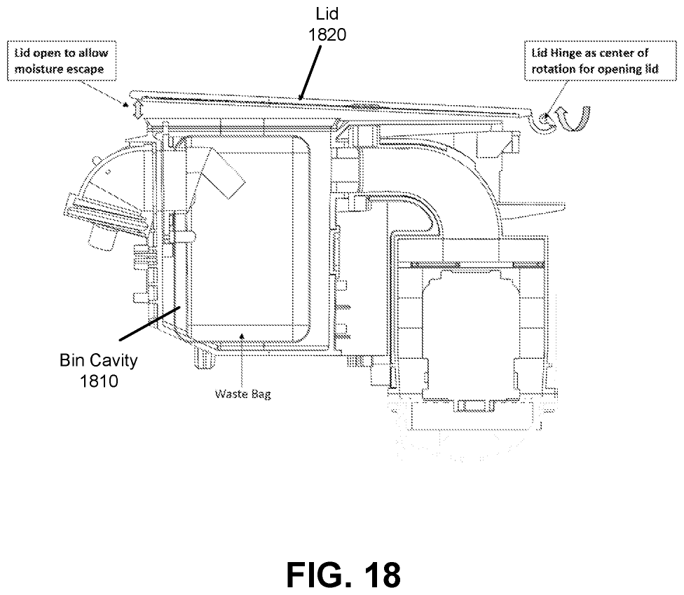

FIG. 18 is a cross-sectional view of the autonomous vacuum with opened lid 1820 for venting of humid air in the bin cavity 1810, according to one or more embodiments. This third solution leverages partially opening of the lid 1820 to allow moisture to escape to the outside environment. The autonomous vacuum may open the lid 1820 when not performing any cleaning operation and there is a need to remove moisture from the bin (e.g., based on sensed humidity within the bin cavity 1810). During the cleaning operation the lid 1810 would remain closed to form an air tight seal to maintain the vacuum pressure within the bin cavity 1810, which aids in cleaning efficiency. During inoperation, to remove the moisture, the autonomous vacuum may open the lid 1820 to a pre-determined angle position, i.e., optimized to keep the angle as low as possible (e.g., a 5 degree opening angle).

The Lid can be actuated open using a few different types of actuators. In one embodiment, a linear actuator is mounted on a stationary part of the chassis (e.g., the bin) and actuates the lid 1820. In another embodiment, a rotary actuator is mounted on a stationary part of the chassis (e.g., the bin) and coupled to a hinge of the lid 1820 to rotate the 1820. In other embodiments, non-contact electromagnetic actuators may actuated the lid 1820 with electromagnetic elements positioned on the lid 1820 and the chassis.

This solution can be implemented in a variety of logics. (1) Keep the lid partially open anytime the cleaning robot is idle. (2) Keep the lid partially open only after a mopping operation is done. Since the robot is able to record information on when was the last time a mopping operation is done, that information can be used to determine when to open the lid. (3) Open the lid depending on the humidity level obtained from the on-board humidity sensor. Anytime the humidity level reaches a certain threshold, the lid can be actuated.

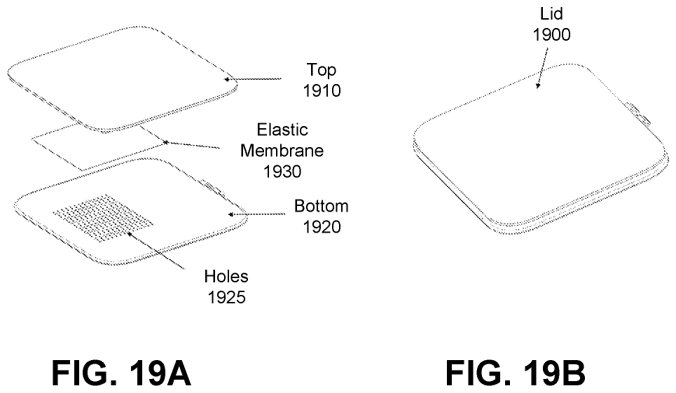

FIG. 19A is an exploded view of a lid 1900 of the autonomous vacuum with an clastic membrane 1930, according to one or more embodiments. FIG. 19B is a constructed view of the lid 1900, according to one or more embodiments. This solution describes a completely passive manner to allow moisture to escape the bin, i.e., leverages operation of the vacuum motor during a cleaning operation. The concept uses a multipart lid with the elastic membrane 1930. There are two rigid parts—top 1910 and bottom 1920—and the clastic membrane 1930 that is attached to the top 1910. The elastic membrane 1930 is a thin film that is impermeable to air. The bottom 1920 contains an array of holes 1925 for the moisture to leave the bin cavity. The number, sizing, and/or positioning of the holes 1925 may be different, i.e., optimized for the case of deforming the elastic membrane 1930 during vacuum operation.

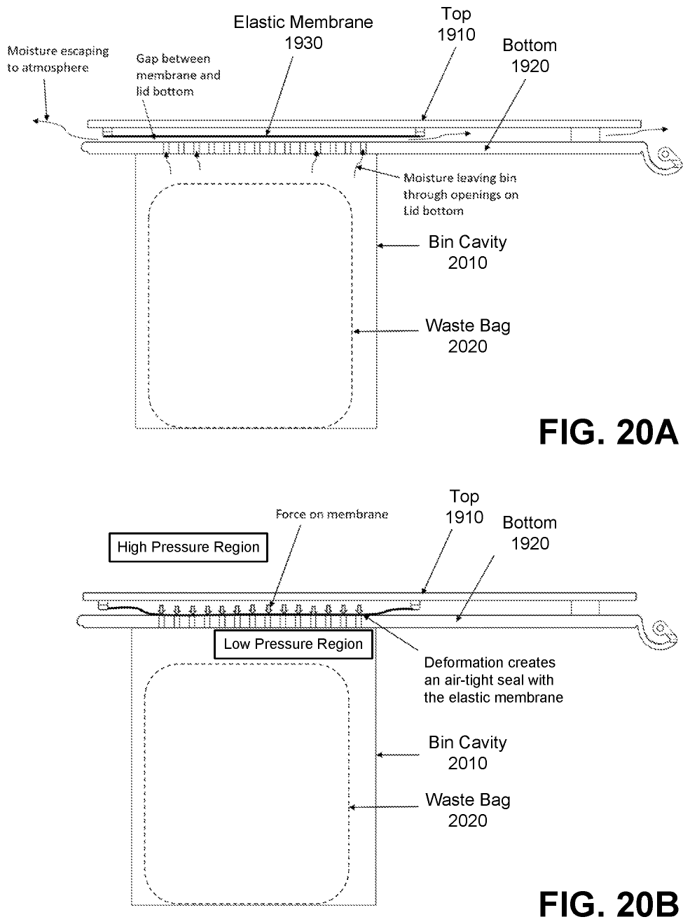

FIG. 20A is a cross-sectional view of the lid 1900 with the elastic membrane 1930 interacting with the bin cavity 2010 of the autonomous vacuum during idle time for venting humid air in the bin cavity 2010, according to one or more embodiments. FIG. 20B is a cross-sectional view of the lid 1900 with the elastic membrane 1930 interacting with the bin cavity 2010 of the autonomous vacuum during operation time, according to one or more embodiments. The elastic membrane 1930 is attached to the top 1910 with one or more pylons disposed on an underside of the top 1910. The top 1910 is attached to the bottom 1920 with another pylon that is of greater height than the pylons used to attach the elastic membrane 1930 to the top 1910.

During inoperation, the elastic membrane 1930 is in its default taut condition with a gap between the elastic membrane 1930 and the bottom 1920 of the lid 1900. In this position, the holes 1925 are unblocked and moisture can escape in a passive manner.

During a cleaning operation, the vacuum motor deforms the elastic membrane 1930 to create an air-tight seal around the holes 1925. The vacuum motor creates a low pressure region in the bin cavity 2010. Since the outside atmosphere is at higher pressure than the bin cavity 2010, the pressure differential generates a force on the elastic membrane 1930 to deform it towards the bottom 1920.

### Anti-Choking Solutions

During the cleaning operation, the waste bag can get stuck into the vacuum motor stack inlet and cause choking of the airflow pathway. Given that suction is the primary mechanism for ingesting debris (wet and/or dry), if the airflow pathway is blocked (wholly or partially), the cleaning performance is drastically reduced. Due to its flexibility, the waste bag typically takes the shape of the bin's internal cavity when a negative pressure is created inside the bin for airflow. Due to the airflow direction and pressure gradient the fabric of waste bag gets pulled into the vacuum stack inlet. This creates the choking of the airflow and drops the airflow down (e.g., down to 10% of maximum potential). This drop in airflow drastically impacts the cleaning performance and the ability of the cleaning robot to continue collecting debris from the floor. Two different solutions may be implemented alone or in conjunction with one another.

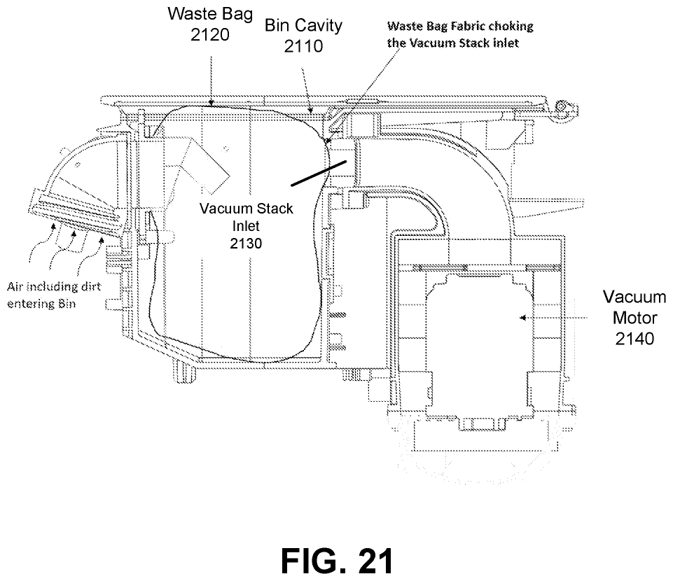

FIG. 21 is a cross-sectional view of the waste bag 2120 choking the vacuum stack inlet 2130, according to one or more embodiments. The waste bag 2120 is installed in the bin cavity 2110. During operation, the vacuum motor 2140 drives a vacuum force that can cause the waste bag 2120 to choke (i.e., block) the vacuum stack inlet 2130, thereby impeding the suction at the cleaning head.

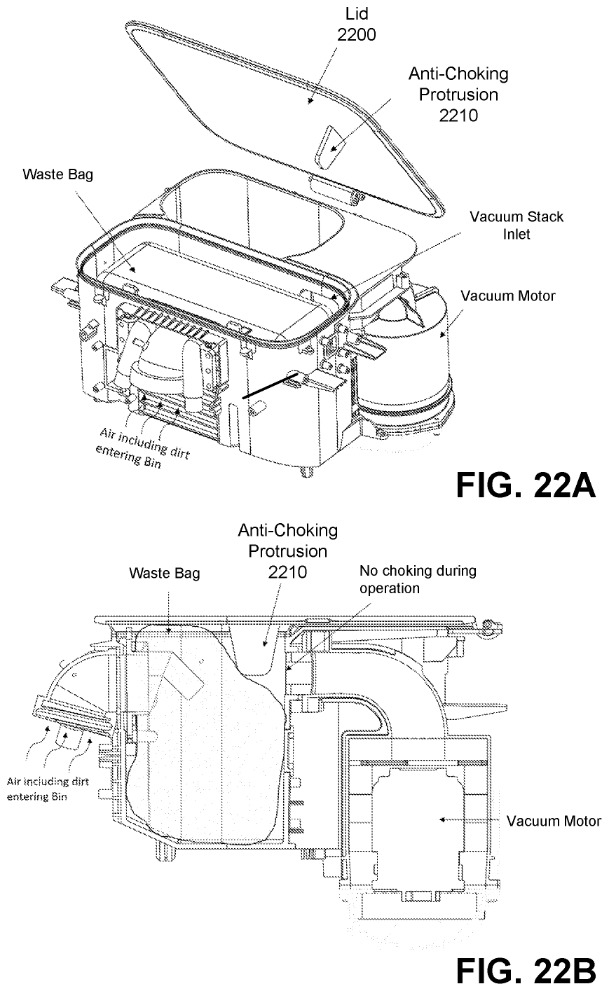

FIG. 22A is a perspective view of a lid 2200 with an anti-choking protrusion 2210, according to one or more embodiments. FIG. 22B is a cross-sectional view of the lid 2200 with the anti-choking protrusion 2210 to mitigate choking of the vacuum stack inlet by the waste bag, according to one or more embodiments. This solution can be implemented in many ways and the idea here is to deflect the bag further away from the vacuum stack inlet so that bag fabric cannot physically reach the inlet.

In one or more embodiments, the anti-choking protrusion 2210 on the lid 2200 would physically push the portion of the waste bag near the vacuum stack inlet so that the portion cannot block the vacuum stack inlet during the operation. The anti-choking protrusion 2210 may be a thin, rigid, and planar element. The orientation of the anti-choking protrusion 2210 may be perpendicular to the vacuum stack inlet, i.e., to minimize its obstruction of the airflow pathway. As shown in FIG. 22B, the anti-choking protrusion 2210 may extend from the bottom of the lid 2200 to extend past the vertical height of the vacuum stack inlet.

This solution solves the issue of bag choking without any change in the surface area of the fabric and the size of the bag. And this in turn ensures there is no impact on amount of dirt being collected.

The solution may also utilize magnet attraction to keep the lid at the preferred position. There could be magnets installed inside the 2200 and the chassis in specific locations to help provide optimum force for sealing. The magnet force ensures the lid 2200 is not pushed open from the upward force created by the waste bag contacting the anti-choking protrusion.

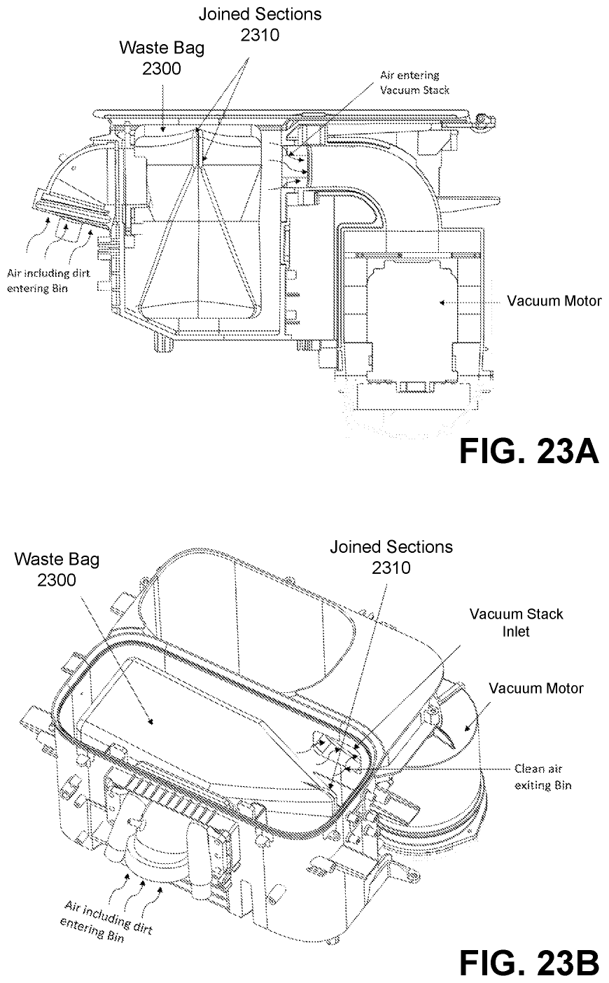

FIG. 23A is a cross-sectional view of a waste bag 2300 with joined sections 2310 to mitigate choking of the vacuum stack inlet by the waste bag 2300, according to one or more embodiments. FIG. 23B is a perspective view of the waste bag 2300 with joined sections 2310 to mitigate choking of the vacuum stack inlet by the waste bag 2300, according to one or more embodiments. A second solution that joins sections of the waste bag 2300 disposed towards the vacuum stack inlet, to prevent choking.

Both the front and back corner of the waste bag (on the top side near vacuum stack inlet) are joined to each other. There are variety of ways to join the sections together including, but not limited to, ultrasonic welding, gluing, stapling, other adhesive, magnets, stitching, etc.

### Water-Absorbing Beads

During the Mopping Cycle, liquid waste is picked up from the ground and collected inside the bag. The collected water can slosh around the waste bag and with time can leak out of the bag. Any additional water that enters the bag at this point, increases the risk of water leaking outside of bag due to all the splashing against the collected water.

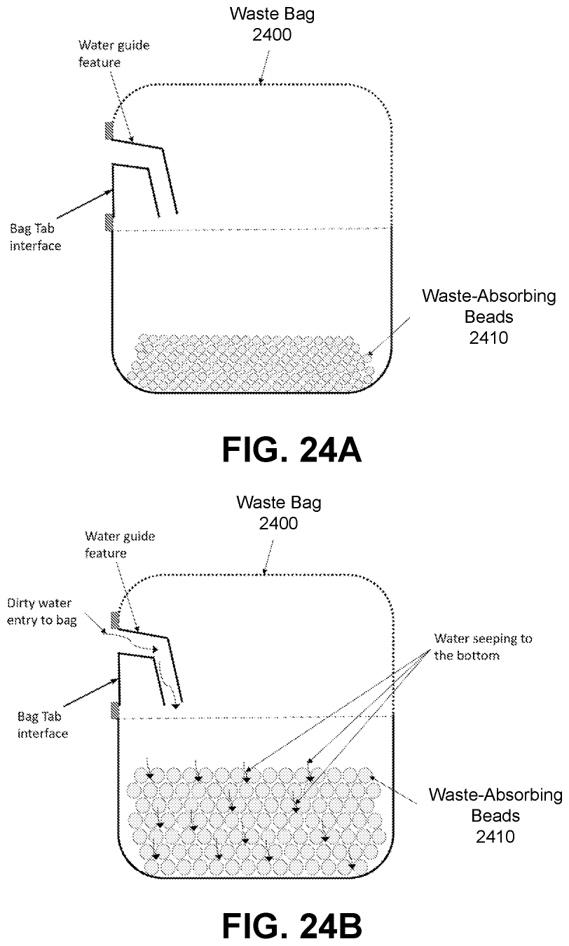

FIG. 24A is a cross-sectional view of the waste bag 2400 with water-absorbing beads 2410, according to one or more embodiments. FIG. 24B is a cross-sectional view of the waste bag 2400 with the water-absorbing beads 2410 in an expanded state upon water absorption, according to one or more embodiments.

To mitigate any leakage, the waste bag may include a water-absorbent material. In one or more embodiments, the waste bag 2400 includes water-absorbing beads 2410. The water-absorbing beads 2410 may be formed with the polymer sodium polyacrylate. It is also known as Super-Absorbent Polymer (SAP) commercially and it has the ability to absorb 100 to 1000 times its mass of water. When dissolved in water, it forms a gel-like thick transparent solution due to ionic interactions of the molecules.

Due to the formation of gel like solution, this would help in preventing the sloshing of water, decreasing the risk of water leakage.

However, there is a particular issue that arises when a granular salt is used. When water is added from the top, the top layer of the salt gets converted into gel and forms a thick layer. This layer creates a barrier and adds resistance to water seeping to the bottom. This results in accumulation of water on the top which is not absorbed by the salt which in turn increases the risk of water leakage due to sloshing and splashing against the collected water.

The solution here uses spherical water-absorbing beads instead of the granular salt. The beads absorb water, but they retain their spherical shape.

FIG. 24A shows the water-absorbing beads 2410 in their initial state without any liquid waste collected into the waste bag 2400 and FIG. 24B shows their state after water absorption. It is observed that the beads grow in size through the absorption of water. This ensures water is no longer present inside the bag in its liquid state and therefore, not susceptible to sloshing or splashing.

One key advantage of using beads comes from its spherical shape and its packing density. For a random packing of equal spheres, the density of packing is around 63.5%, that means around 36.5% is free space. Due to this free space, water can easily find its way towards the bottom of the bag. Even after the spheres increase in size, the packing density still remains same and hence the ability of water to seep towards the bottom remains the same. This helps in utilization of deeper layers of the beads at the bottom and thus increases the effectiveness of water absorbents.

### Anti-Odor and Anti-Microbial Solutions

When liquid waste is ingested into the waste bag, the liquid waste (in conjunction with the dry waste) can, over time, foster microbial growth, leading to foul odors and/or unhygienic conditions. To combat such unpleasantries, in one or more embodiments, the waste bag can implement one or more anti-odor and/or anti-microbial solutions. A first solution implements a chemical agent in the waste bag to counter microbial growth and/or to counter foul odor. A second solution implements a chemical agent in the solvent. As the solvent is dispersed and the liquid waste (derived from the solvent) is ingested into the cleaning head, the solvent with the chemical agent can clean the channels leading to the waste bag.

FIG. 25A is a front perspective view of the waste bag 2500, according to one or more embodiments. FIG. 25B is a rear perspective view of the waste bag 2500 of FIG. 25A, according to one or more embodiments. FIG. 25C is a front cutaway view of the waste bag 2500 of FIG. 25A, according to one or more embodiments. The waste bag 2500 includes the non-permeable portion 2510, the permeable portion 2520, the bag tab 2530, and the rigid cage 2540.

The non-permeable portion 2510 is non-permeable to at least liquid. The permeable portion 2520 is permeable to at least air. The non-permeable portion 2510 and the permeable portion 2520 form an enclosed cavity for holding dry waste, liquid waste, or a combination thereof. Waste enters the waste bag 2500 through an opening in the permeable portion 2520. The bag tab 2530 is a rigid structure that connects to the permeable portion 2502 around the opening. The bag tab 2530 may include one or more holes to connect to one or more channels that funnel waste into the waste bag 2500. The waste bag 2500 includes one or more chemical agents to counteract odor and microbial growth in the waste stored in the waste bag 2500. Example chemical agents include: benzethonium chloride, activated carbon powder, zeolites, citric acid, benzoic acid, sodium bicarbonate,

The rigid cage 2540 is coupled to an interior surface of the non-permeable portion 2510 (or the bottom portion of the waste bag 2500). The rigid cage 2540 holds the chemical agent. In the embodiment shown in FIG. 25C, the rigid cage 2540 includes a back wall coupled to the inner surface of the non-permeable portion 2510, side walls extending from the back wall towards an interior of the waste bag 2500, and a grated wall with perforations attached to the side walls. The walls form a cage to hold the chemical agent. The grated wall permits liquid waste in the waste bag 2500 to interact with the chemical agent stored in the rigid cage 2540. In some embodiments, the rigid cage 2540 may be divided into multiple chambers to permit staged release of the chemical agent. For example, the rigid cage 2540 may be divided into a top chamber and a bottom chamber, wherein the liquid waste first interacts with the chemical agent stored in the bottom chamber. As the liquid waste level rises to the top chamber, then the liquid waste interacts with the chemical agent stored in the top chamber.

In other embodiments, the waste bag 2500 may include one or more sachets (not illustrated) that are tethered or placed into the interior of the waste bag 2500. The sachet may be tethered by one or more strings connected to both the inner surface of the permeable portion 2520 and an outer surface of the sachet. The sachets may be formed of a dissolvable material, e.g., water soluble paper. The sachets may hold the chemical agent for counteracting odor and/or microbial growth. The sachets may also hold the water-absorbing material, e.g., the water absorbing beads. In some embodiments, the chemical agent absorbs excess moisture and odor from the air inside the waste bag 2500. In such embodiments, the chemical agent may be suspended above the waste via the sachets.

In one or more embodiments, the autonomous vacuum may implement a fragrance dispersion system. Such fragrance dispersion system may include a tank to hold fragrance and a spraying mechanism for dispensing the fragrance. The fragrance dispersion system may be located in the waste bag cavity, e.g., proximate to the waste bag.

In one or more embodiments, the autonomous vacuum may implement other sterilization technologies to mitigate odor and/or microbial growth. For example, the autonomous vacuum may implement an ultraviolet emitter to emit ultraviolet radiation to sterilize the waste collected in the waste bag. In another example, the autonomous vacuum may implement a photocatalytic oxidizer, ionizing purifiers, or other sterilization systems. The photocatalytic oxidation involves using a photocatalyst, e.g., titanium dioxide (TiO2), which, when exposed to an appropriate wavelength of radiation (e.g., ultraviolet radiation), becomes activated and can oxidize organic materials in the surrounding environment into less harmful substances like carbon dioxide and water. An ionizing purifier is an air purification device that uses ions to remove particulates, microbes, and odors from the air. The device creates negative ions and releases them into the air. These negative ions bind with positively charged airborne particles, e.g., dust, pollen, smoke, and allergens, thereby giving them a negative charge. Once charged, these particles can be attracted to nearby surfaces or even each other. When the particles cluster together in this way, they become heavier, making it easier for them to fall out of the air and be filtered out or collected on a plate inside the ionizer.

FIG. 26 is a cross-sectional view of a solvent pathway in the autonomous vacuum, according to one or more embodiments. The autonomous vacuum includes a solvent tank 2600 disposed within the chassis. One or more liquid channels connect the solvent tank 2600 to the cleaning head's solvent dispersion system 2610. The solvent dispersion system 2610 dispenses the solvent into a cleaning environment, i.e., external to the cleaning head. The mop roller 2620 cleans the cleaning environment with the solvent and ingests the liquid waste. The liquid waste enters the cleaning head and is funneled into the waste bag 2640 via one or more channels in the connection assembly 2630.

To counteract odor and microbial growth, the solvent tank 2600 may store solvent with one or more chemical agents to aid in sterilizing the waste. The chemical agents may be, for example, detergents, fragrances, silver-ion-infused ceramic balls, or some combination thereof.

Silver ions (Ag+) are positively charged particles of silver. When infused into materials like ceramic balls, they act as a powerful antimicrobial agent. The silver ions are released slowly when in contact with moisture. These ions can then interact with bacterial cells in multiple ways: 1. Cell Membrane Disruption: Silver ions can bind to and penetrate the cell walls of bacteria, causing structural changes in the cell membrane that lead to the cell's rupture or increased permeability, ultimately killing or disabling the bacteria. 2. Interference with Metabolic Processes: Once inside the cells, silver ions can disrupt key enzyme functions, hindering the cell's ability to produce energy, which is vital for the survival of the microorganism. 3. Inhibition of Reproduction: Silver ions can interfere with the replication of DNA in microbial cells, preventing them from multiplying and spreading.

In one or more embodiments, silver-ion-infused ceramic balls may be positioned in different positions of the autonomous vacuum. As one example, the ceramic balls may be placed within the water tank. In another example, the ceramic balls may be integrated into the solvent dispersion or the waste collection systems. As the solvent with the silver ions are routed through the solvent pathway, the silver ions disinfect the components of the solvent pathway. Advantages to using silver ions include: non-toxicity, gentle on cleaning surfaces, long-lasting, environmentally friendly, etc.

### Waste Bag Fullness Detection

In some embodiments, to further mitigate leakages, the autonomous vacuum includes sensors for detecting fullness of the waste bag. Some sensors may be used for detecting liquid waste fullness, whereas other sensors may be used for detecting dry waste fullness. In other embodiments, the sensors measure the aggregate waste fullness (i.e., dry and liquid waste combined). Based on the detected fullness, the autonomous vacuum can implement logic to prompt the user to remove the waste bag when needed. This can help to improve the overall efficiency and effectiveness of the cleaner, as well as eliminate the need for users to constantly monitor the bag to see when it is full.

In principle, a bag will be considered full when one or more conditions are met. Other embodiments may include additional, fewer, or different fullness conditions. First condition: the bag is filled with waste up to a point where the collected debris starts overflowing back into the channels of the connection assembly and/or the cleaning head. This results in a bad user experience and/or potential malfunction of the robot. Second condition: the bag is clogged with dust and airborne particles causing an increase in the airflow resistance and reducing the cleaning efficacy beyond a usable threshold. Third condition: decomposition of debris inside the bag when left in the bag for an extended period of time resulting in bag odor. Fourth condition: the liquid waste has reached a maximum fill line (e.g., which may be determined based on the ratio of the non-permeable portion to the permeable portion of the waste bag).

In some embodiments, the autonomous vacuum can detect the fullness of a waste bag with one or more sensors. These sensors can be placed inside the bag itself, or within the autonomous vacuum's bin cavity. When the sensors detect that the waste bag is full, it can alert the user through a variety of means. This could include a visual or audible notification on the cleaner itself, or a notification sent to the user's smartphone or another device. In some embodiments, given that the autonomous vacuum has a good semantic understanding of its environment, it may also choose to navigate to the nearest trash can inside the house. The user can then take appropriate action, such as disposing of the full bag and inserting a new one to ensure that the cleaner continues to operate effectively.

In some embodiments, the autonomous vacuum can detect the fullness of a waste bag is through one or more machine learning algorithms. These algorithms can be trained to recognize the pattern of waste accumulation in the bag over time, and to predict when it is likely to reach capacity. When this prediction is made, the floor cleaner can send an alert to the user to let them know that it is time to change the bag.

It is important that any combination of these solutions when implemented, result in a reliable and accurate assessment of the level of the bag at any point in time. There are two important failure modes to be avoided: (1) if the sensor overestimates the level of the bag, it would result in sub-optimal usage of the bag and increased wastage, and (2) on the contrary, if the bag underestimates the level of waste inside the bag, it may result in either reduced cleaning efficacy or waste overflowing out of the bag into undesired areas.

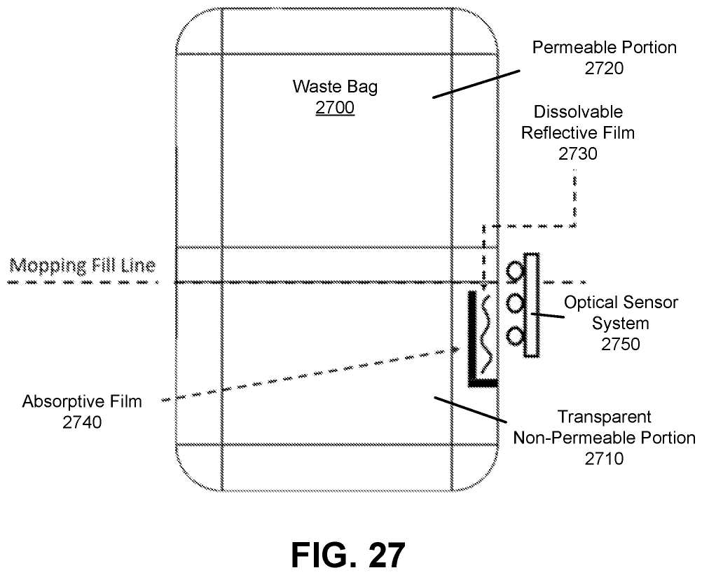

FIG. 27 is a cross-sectional view of an optical sensor system 2750 for liquid waste fullness detection, according to one or more embodiments. The optical sensor system 2750 includes at least a light receiver and a light emitter. In some embodiments, the optical sensor system 2750 further includes an optics block, other optical elements, or some combination thereof.

The light emitter is configured to emit light from the bin cavity towards the waste bag 2700. The light emitter may emit continuous light or pulses of light. The light emitted may be in the visible spectrum, in the infrared spectrum, in the ultraviolet spectrum, another electromagnetic spectrum, or some combination thereof. The light emitter may include a plurality (e.g., three) infrared emitters whose wavelength is centered around 940 nm and whose spectral bandwidth and viewing angle are narrow. A load switch may be used to pulse the light emitters when a bag fullness measurement is being performed, effectively minimizing system power draw and thermal implications.

The light receiver is configured to measure an intensity of light incident on the light receiver. In some embodiments, the light receiver is positioned adjacent to the light emitter. The light receiver may be positioned in the bin cavity near a mopping fill line of the waste bag. The light receiver may include a photosensitive element, an analog front-end (AFE), and an Analog-Digital converter (ADC). As the photosensitive element, a plurality (e.g., three) of photodiodes are used which via the photoelectric effect product an electrical current proportional to the light intensity incident on the photodiodes. The photodiodes are chosen to match the viewing angle of the light emitters, minimize response time, and optimize for linearity and relative spectral sensitivity around 940 nm to minimize noise sources. The AFE may include a transimpedance amplifier, biasing, and other filter elements performs current to voltage conversion, amplification, and active and passive filtering. Filters were designed to optimize response time while eliminating relevant noise sources. An ADC is used to convert the analog output voltage of the AFE to a digitized result which can then be read and interpreted by the microprocessor.

The optical sensor system 2750 system works by emitting light and measuring the amount of reflected light. The optical sensor system 2750 further implements a dissolvable reflective film 2730 and an absorptive film 2740 positioned within the waste bag 2700 and adjacent to the first senor optical system 2750 when the waste bag 2700 is installed into the bin cavity. When the bag is empty, the light emitted by the light emitter will be reflected back by the dissolvable reflective film 2730 to the light receiver with minimal obstruction. As the bag begins to fill up with liquid waste, the amount of light which reflects back to the light receiver will remain unaffected until a certain water level (e.g., up until the bottom of the dissolvable reflective film 2730). As the water level reaches the dissolvable reflective film 2730, the dissolvable reflective film 2730 interacts with the water, which dissolves the dissolvable reflective film 2730. As the dissolvable reflective film 2730 is dissolved into the water, the absorptive film 2740 is disoccluded. The absorptive film 2740 may be colored black. By nature of the black color and other surface properties, this absorptive film 2740 will minimally reflect light emitted by the light emitter. The light receiver measures the decreased reflection of light, and the system can detect that the waste bag 2700 is full based on that sensed light data.

In other embodiments, the optical sensor system 2750 may utilize a dissolvable absorptive film occluding a reflective indissolvable film. When the bag is empty, the emitted light is absorbed by the absorptive film. The light receiver detects little to no reflected light. The system can deem the bag empty based on the nominal reflections. When the liquid level interacts with the dissolvable film, the liquid dissolves the dissolvable film, disoccluding the reflective indissolvable film. Emitted light is then reflected off the reflective indissolvable film back to the light receiver. The light receiver measures the higher amount of reflection. The system can deem the bag full (or quantify fullness) based on the sensed light data.

The use of infrared light to detect the level of the bag has several key advantages. For one, the system can detect the bag fullness without needing to physically place a sensor inside the bag. This circumvents the need for another dynamic seal on the bag around the sensor and for space in the bag for placement of the sensor, which the debris could otherwise occupy. For two, light in the infrared spectrum (e.g., with a nominal wavelength of 940 nm) is not visible to the human eye, so it does not disturb the user during the cleaning or bag insertion and/or removal process.

In one or more embodiments, the optical sensor system 2750 utilizes three emitter-receiver pairs. This would entail three light emitters and three light receivers. The three pairs provide for critical redundancy, given that the waste bag 2700 is a flexible component. Due to its flexibility, the mopping fill line falls within a wide range of vertical positions rather than a specific position. With three emitter-receiver pairs, the system is immune to the potential positional variations. Three emitter-receiver pairs may also all the system to quantify the fullness of the bag. For example, sensed light at the bottom-most emitter-receiver pair indicates the liquid waste level reaching a first level (e.g., 80% full). When the liquid waste level reaches the middle emitter-receiver pair, the system may determine that the bag is at a second level (e.g., 90% full). Finally, when the liquid waste level reaches the top-most emitter-receiver pair, the system may determine that the bag is at a third and final level (e.g., 100% full).

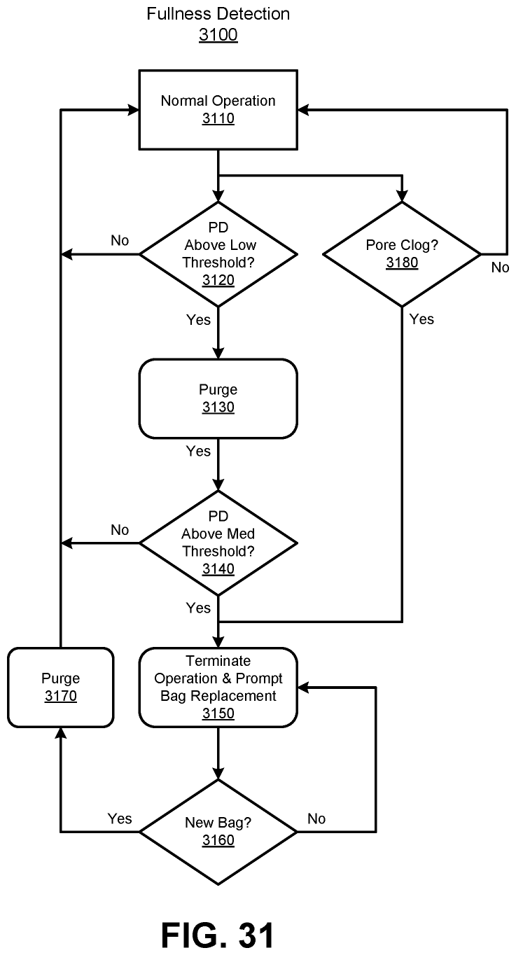

The system's fullness detection algorithm may be threshold-based. When all three sensor elements read below specified thresholds, the system deems the waste bag to be full. A certain amount of hysteresis is important to avoid oscillations in the bag full indication. This is implemented with software controls and two thresholds. Once all three emitter-receiver pairs read below the first threshold, a “purge” is executed. A purge consists of running the vacuum at an elevated speed. This tends to clear debris pathways, and redistribute bag contents in a predictable manner. The redistribution of the bag contents also tends to decrease the photodiode sensor element readings further. Once the purge is complete, the second threshold, which is slightly higher than the first, is used to make the final decision on whether the bag is indeed full. If readings exceed this second threshold after the purge, it will be interpreted as a false positive, and the system will resume regular operation. The way a bag folds and sits within the bin cavity can have a significant effect on sensor readings. To address this, an inflation cycle, consisting of running the vacuum motor to drive high airflow, may be run every time a new bag is detected. This inflation cycle expands the bag to a predictable shape, ensuring consistency between different waste bags. This logic is further described in FIG. 31.

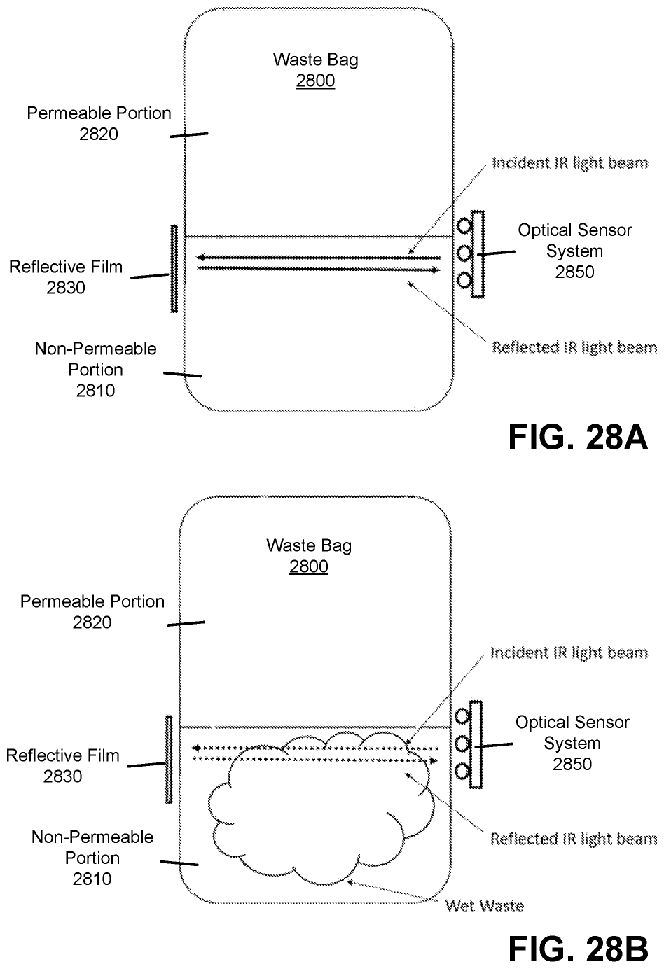

FIG. 28A is a cross-sectional view of an optical sensor system 2850 for liquid waste fullness detection, according to one or more embodiments. FIG. 28B is a cross-sectional view of the second optical sensor system 2850 for liquid waste fullness detection, according to one or more embodiments. The optical sensor system 2850 is an embodiment of the optical sensor system 2750. In the embodiment of FIGS. 28A & 28B, the waste bag 2800 includes a reflective film 2730. The reflective film 2730 is disposed on a first side of the waste bag 2800, whereas the optical sensor system 2850 is adjacent to a second side of the waste bag 2800, that is opposite the first side. The reflective film 2730 may be positioned close to the fill line. Here, the light emitted by the optical sensor system 2850 passes through the waste bag 2800 to reflect off the reflective film 2830. In FIG. 28A, when the bag is empty, the emitted light passes unobstructed from the light emitter(s), through the waste bag 2800, reflecting off the reflective film 2830, back through the waste bag 2800, and incident on the light receiver(s). In FIG. 28B, when the bag is full (or filling), the emitted light is obstructed by the liquid waste and contaminants when passing through the waste bag 2800, diminishing the amount of light that passes through the waste bag 2800. The system can determine bag fullness by detecting the diminished amount of reflected light (e.g., as sensed by the light receiver).

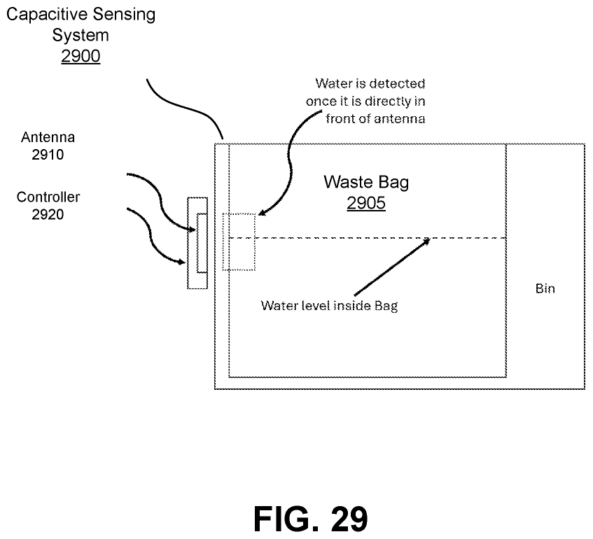

FIG. 29 is a cross-sectional view of a capacitive sensor system 2900 for liquid waste fullness detection, according to one or more embodiments. The capacitive sensor system 2900 includes an antenna 2910 and a controller 2920.

The antenna 2910 includes one or more capacitive sensing electrodes. The antenna 2910 is positioned along a side wall of the waste bag cavity in proximity to the waste bag 2905. An electrical current is run through the capacitive sensing electrodes generating an electrical field proximate to the electrodes in the waste bag cavity. As objects draw near to the electrodes, a change in the electrical field can be measured as a change in the surface capacitance. The electrodes may further be arrayed, to provide insight into changes of capacitance at different points in the array of electrodes. This can provide useful in quantifying fullness of the liquid waste in the waste bag 2905. The antenna 2910 may be positioned at a height that corresponds to the liquid waste full level of the waste bag 2905. For example, with a semi-waterproof waste bag, the liquid waste full level may be the coupling point of the non-permeable portion and the permeable portion. In one or more embodiments, the capacitive sensing system 2900 may include multiple antennae placed at differing positions around the waste bag cavity. For example, there may be one antenna on one wall of the waste bag cavity, and another antenna on another wall of the waste bag cavity.

In one or more embodiments, the controller 2920 detects the liquid waste fullness based on the capacitive sensing. When an empty bag is installed, the controller 2920 can determine a baseline capacitance. As the bag is filled with liquid and/or dry waste, the controller 2920 receives capacitance measurements from the antenna. The controller 2920 may compare the capacitance measurements to the baseline capacitance to determine the liquid waste fullness. In some embodiments, the controller 2920 may further provide a quantification level of the liquid waste, e.g., 80%, 85%, 90% full, etc.

In one or more embodiments, the capacitive sensing system 2900 can implement calibration processes to ensure accuracy over time. For example, if the capacitive sensing system 2900 determines that the baseline capacitance measured when new waste bags are placed into the cavity changes over time, then the capacitive sensing system 2900 may determine there to be a calibration error. In another example, if the capacitive sensing system 2900 detects decreases in capacitance after installation of a new waste bag, then the capacitive sensing system 2900 may note another calibration error.

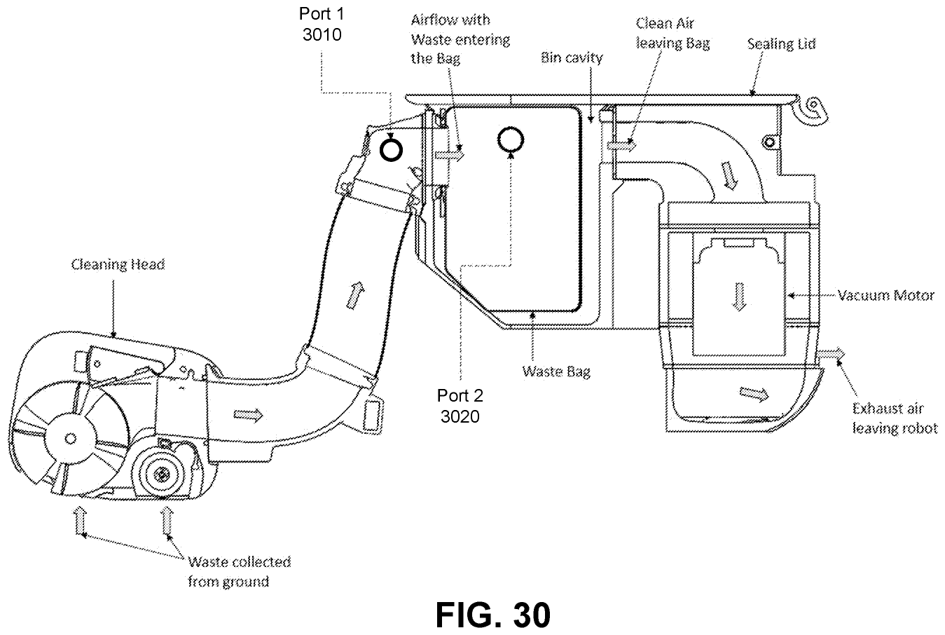

FIG. 30 is a cross-sectional view of the airflow pathway in the autonomous vacuum with ports for pressure sensing, according to one or more embodiments. In this embodiment, Port 1 3010 may be positioned in the channel of the connection assembly, and Port 2 3020 may be positioned in the bin cavity. In other embodiments, additional ports for pressure sensing may be implemented at additional points along the airflow pathway.

In the case of sweeping where dry waste is getting picked up by the cleaning system, maintaining an unhindered airflow path is important for maintaining high cleaning efficacy. This can be detected using multiple differential pressure sensors which measure the difference in pressure between a port location and the ambient pressure. One of the main reasons that the pores of a waste bag can become clogged is the presence of dust and other particles in the air. These particles can settle on the surface of the bag and can eventually work their way into the pores, causing them to become blocked. This can happen over time, as the bag is exposed to the elements and to the dust and particles in the air. In addition to dust and particles, other substances, such as oil and grease, can also cause the pores of a waste bag to become clogged. These substances can coat the pores, making it difficult for waste to pass through them. While the bag might not seem visually full, pores getting blocked beyond a certain point will result in poor cleaning efficacy.

The region bound by the two pressure ports 3010 and 3020 is the bag fullness detection area. The pressure ports are connected to pressure sensors feeding pressure information to the controller. In a bag not-full state, there will be minimal difference between the pressure read at Port 1 3010 and the pressure read at Port 2 3020. However, if the bag is full (i.e., the dry waste is hindering vacuum airflow through the permeable portion of the waste bag) up to a point where the resistance to airflow is above a certain threshold, a pressure drop will be reflected across the two ports, such that the pressure of port 2 is much lower than the pressure of port 1, meaning a large pressure differential. The system can determine the dry fullness (binary or quantified) of the waste bag based on the pressure differential.

The pressures sensor may be monitored by an onboard controller which communicates with these sensors via a digital communication interface. The controller can also handle the autonomous vacuum's state machine, cleaning and fault logic, system shutdown, notification of the user, or some combination thereof. A communication multiplexer is used to allow both pressure sensors to communicate with the controller via a single communication bus.

The logic for detecting clogged pores is similar to the bag fullness logic in the case of sweeping. Once the difference in pressure in the bag fullness detection area is above a certain value, a bag will be considered full. In most cases, it should be possible to differentiate between the pore-clogging behavior and the bag being full in the sweeping scenario (e.g., leveraging the optical sensor system for liquid waste fullness detection).

FIG. 31 is a flowchart 3100 of dry waste fullness detection, according to one or more embodiments. The flowchart may be performed by a controller of the autonomous vacuum, e.g., in conjunction with other sensors of control systems of the autonomous vacuum. In other embodiments, some or all steps may be performed by another computing system.

At step 3110, the controller performs normal operation for the autonomous vacuum.

The controller receives the pressure readings from the pressure sensors. In the embodiment of FIG. 30, there may be two ports for pressure readings.

At step 3120, in dry fullness detection, the controller evaluates whether the pressure differential between the two ports is above a low threshold. The low threshold may indicate that the cleaning efficiency has dropped significantly to warrant further assessment.

If the pressure differential is not above the low threshold, the controller returns to normal operation 3110.

At step 3130, if the pressure differential is above the low threshold, the controller performs a purge. A purge involves running the vacuum motor at a high speed (i.e., high revolutions per minute) to create a larger-than-normal-operation suction power.

At step 3140, The controller then evaluates whether the pressure differential between the two pressure readings is above a medium threshold. The medium threshold may be higher than the low threshold, indicating that the bag is dry-full.

If the pressure differential is not above the medium threshold, then the controller can deem the fullness event as a false positive, and return the autonomous vacuum to normal operation 3110.

At step 3150, if the pressure differential is above the medium threshold, the controller can determine that the bag is dry-full. In determining that the bag is dry-full, the controller can terminate operation and prompt bag replacement. Prompting bag replacement may entail providing the user with any manner of notification (e.g., visual notification on a display, audio notification, mobile device notification, etc.). In some embodiments, the controller further evaluates the liquid waste fullness, e.g., via the optical sensor system.

At step 3160, the controller evaluates whether a new bag has been replaced for the old bag. The controller may perform checks of the liquid waste fullness detection and dry fullness detection, to assess whether the replaced bag is indeed new (or, at the very least, not full).

If the bag is deemed to be not new, the controller may return to step 3150 maintaining inoperation and reprompting the user to replace with a new bag.

If the bag is deemed to be new, the controller can return to normal operation 3110. In one or more embodiments, at step 3170, the controller can perform another purge of the new bag, e.g., to prepare the bag for normal operation.

At step 3180, the controller may evaluate whether the pores are clogged. The controller may assess whether one or more of the ports is clogged and/or whether there is a higher than a critical threshold pressure differential between ports.

At step 3150, if one or more of the ports is clogged and/or the airflow pathway is clogged, then the controller can terminate operation and prompt bag replacement (or to perform other remedial measures to address the clog).

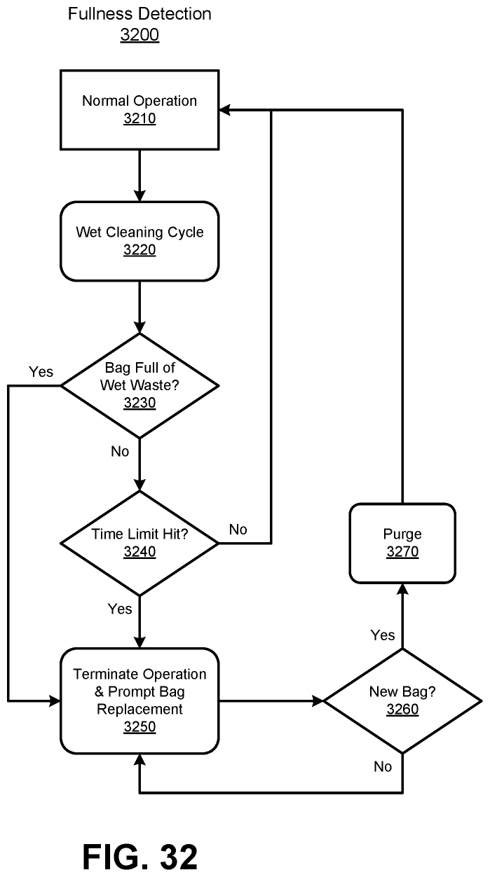

FIG. 32 is a flowchart of liquid waste fullness detection 3200, according to one or more embodiments. The flowchart may be performed by a controller of the autonomous vacuum, e.g., in conjunction with other sensors of control systems of the autonomous vacuum. In other embodiments, some or all steps may be performed by another computing system.

When waste is left in a trash can for an extended period of time, it can create a bad smell. This is because the waste begins to decompose and release gases, such as methane and sulfur dioxide, which have a distinctive odor. One of the main reasons that waste begins to decompose and release gases is that it is a breeding ground for bacteria and other microorganisms. These organisms break down the organic matter in the waste, releasing gases in the process. The gases can then become trapped inside the trash can, causing the unpleasant smell to build up. The smell can also be exacerbated by the presence of certain types of waste, such as food waste and yard waste. These materials are more likely to break down quickly and release gases, leading to a stronger odor.

A simple time-based approach can be used to detect this case. Once the bag has been used for a cleaning cycle, if the time elapsed since the last bag replacement exceeds a given threshold (say a month), the user can be alerted to replace the bag.

At step 3210, the controller performs normal operation of the autonomous vacuum.

At step 3220, the controller performs a wet cleaning cycle 3220. The wet cleaning cycle 3220 generally will yield liquid waste in the waste bag.

At step 3230, the controller performs liquid waste fullness detection of the waste bag. This may be accomplished with the optical sensor system described in FIGS. 27, 28A, and 28B.

If the bag is full, at step 3250, the controller terminates operation and prompts bag replacement.

If the bag is not full, at step 3240, the controller assesses whether a time limit on bag use has been hit. For example, the time limit may be one week, two weeks, three weeks, four weeks, one month, etc. The time limit may be set by the user, or determined by the controller based on wet cleaning cycles. For example, a lower time limit may be used if wet cleaning cycles are frequent. Contrarily, a higher time limit may be used if wet cleaning cycles are infrequent.

If the time limit has hit, at step 3250, the controller terminates operation and prompts bag replacement.

If the time limit has not yet hit, at step 3210, the controller returns to normal operation.

After terminating operation and prompting bag replacement, at step 3260, the controller assesses whether a new bag has been placed into the bin cavity. The controller may use the dry fullness detection, the liquid waste fullness detection, or some combination thereof to assess.

If the controller detects there to a new bag, at step 3210, the controller returns to normal operation.

If the controller detects there that the bag was not replaced, or that the replaced bag is still full (or nearly full), then at step 3250, the controller maintains inoperation and may reprompt the user to replace the bag.

### EXAMPLE CLAUSES

Clause 1. A semi-waterproof waste bag for an autonomous vacuum, the waste bag comprising: a non-permeable portion composed of a waterproof material and structured to hold liquid waste from the autonomous vacuum; and a permeable portion composed of an air-permeable material to filter out dry contaminants from air, the permeable portion affixed atop the non-permeable portion, and the permeable portion having a first opening for waste to enter the semi-waterproof waste bag.

Clause 2. The semi-waterproof waste bag of clause 1, wherein the first opening is for dry waste from a main channel of the autonomous vacuum.

Clause 3. The semi-waterproof waste bag of clause 2, wherein the permeable portion includes one or more secondary openings for liquid waste to enter the semi-waterproof waste bag from one or more secondary channels of the autonomous vacuum.

Clause 4. The semi-waterproof waste bag of clause 3, wherein the semi-waterproof waste bag further comprises: one or more guide vanes disposed within the semi-waterproof waste bag to guide liquid waste entering the semi-waterproof waste bag away from the permeable portion and towards the non-permeable portion.

Clause 5. The semi-waterproof waste bag of clause 1, further comprising: a bag tab formed of a rigid material, the bag tab affixed to the permeable portion, the bag tab configured to slot into a tab receiver of the autonomous vacuum to secure the semi-waterproof waste bag in a bin cavity of the autonomous vacuum.

Clause 6. The semi-waterproof waste bag of clause 5, wherein the bag tab includes an opening that aligns with the first opening of the permeable portion.

Clause 7. The semi-waterproof waste bag of clause 6, wherein the bag tab further comprises a gasket of annular shape formed of a compliant material that aligns to the opening of the bag tab, wherein the gasket forms an air-tight seal between the semi-waterproof waste bag and the bin cavity of the autonomous vacuum when the semi-waterproof waste bag is secured in the bin cavity of the autonomous vacuum.

Clause 8. The semi-waterproof waste bag of clause 7, wherein the bag tab is substantially planar, wherein the bag tab includes a bag tab bump on a back face of the bag tab that faces the permeable portion of the semi-waterproof waste bag, and wherein a bump on the tab receiver applies a lateral force on the bag tab bump when the semi-waterproof waste bag is secured in the bin cavity of the autonomous vacuum.

Clause 9. The semi-waterproof waste bag of clause 1, further comprising: a film composed of a waterproof material, disposed along a wall of an interior of the semi-waterproof waste bag, affixed to an underside of a top face of the permeable portion, and substantially coextensive to the top face of the permeable portion.

Clause 10. The semi-waterproof waste bag of clause 9, wherein the film is affixed to the underside of the top surface of the permeable portion by: ultrasonic welding, adhesive, stapling, or stitching.

Clause 11. The semi-waterproof waste bag of clause 1, wherein two corners of the permeable portion of the waste bag are joined together.

Clause 12. The semi-waterproof waste bag of clause 11, wherein two corners of the permeable portion of the waste bag are joined together by: ultrasonic welding, adhesive, stapling, magnetic coupling, or stitching.

Clause 13. The semi-waterproof waste bag of clause 1, further comprising: a reflective film composed of a light-reflective material and coupled to the non-permeable portion proximate to an interface between the non-permeable portion and the permeable portion, wherein the reflective film reflects back light emitted by an optical sensor system.

Clause 14. The semi-waterproof waste bag of clause 13, further comprising: an absorptive film composed of a light-absorptive material and coupled to an interior face of the reflective film, and wherein the reflective film is dissolvable by water.

Clause 15. The semi-waterproof waste bag of clause 1, further comprising: a rigid cage coupled to an inner surface of the non-permeable portion, the rigid cage including one or more openings to an interior of the waste bag and structured to hold a chemical agent for sterilization of waste in the waste bag, or a sachet tethered to an inner surface of the permeable portion, the sachet formed of a permeable material and storing a chemical agent for sterilization of air in the waste bag.

Clause 16. An autonomous vacuum comprising: a vacuum stack including a vacuum motor configured to generate a vacuum force; a bin cavity formed to fit a waste bag, wherein the bin cavity includes: a cavity opening that connects to a cleaning head of the autonomous vacuum where waste is ingested into the autonomous vacuum, a tab receiver including two receiving elements positioned at a width of a bag tab of the waste bag, wherein the tab receiver is positioned to be centered around the cavity opening, and a vacuum stack inlet that connects the bin cavity to the vacuum stack.

Clause 17. The autonomous vacuum of clause 16, further comprising: a bag-bin seal formed of a compliant material and positioned around the cavity opening of the bin cavity.

Clause 18. The autonomous vacuum of clause 17, wherein the bag-bin seal includes: a planar portion of annular shape, wherein an inside diameter of the annular shape matches to the diameter of the cavity opening; an inner ring that is substantially perpendicular to the planar portion; and an outer ring of arcuate shape that bends towards a center axis of the bag-bin seal.

Clause 19. The autonomous vacuum of clause 18, wherein the inner ring is of varying thickness.

Clause 20. The autonomous vacuum of clause 18, wherein the outer ring is of substantially uniform thickness.

Clause 21. The autonomous vacuum of clause 18, wherein the outer ring is of shorter length than the inner ring.

Clause 22. The autonomous vacuum of clause 18, wherein the inner ring extends into the waste bag when the bag tab of the waste bag is secured into the tab receiver.

Clause 23. The autonomous vacuum of clause 18, wherein the outer ring forms an air-tight seal when the bag tab of the waste bag is secured into the tab receiver.

Clause 24. The autonomous vacuum of clause 16, wherein each receiving element of the tab receiver comprises: one or more bumps that interact with a corresponding bump on the bag tab of the waste bag to secure the bag tab to the tab receiver.

Clause 25. The autonomous vacuum of clause 24, wherein the bumps of the receiving elements further apply a lateral force to the bag tab.

Clause 26. The autonomous vacuum of clause 16, further comprising: a lid positioned to cover the bin cavity to form an airtight seal during operation of the vacuum motor.

Clause 27. The autonomous vacuum of clause 26, wherein the lid comprises: a top portion; a bottom portion affixed to the top portion and including an array of holes that connect to the bin cavity when the lid covers the bin cavity; and an elastic membrane affixed to the top portion, positioned in between the top portion and the bottom portion, aligned to the array of holes, and configured to deform to block the array of holes during operation of the vacuum motor.

Clause 28. The autonomous vacuum of clause 26, wherein the lid comprises: an anti-choking protrusion positioned to extend into the bin cavity in front of vacuum stack inlet.

Clause 29. The autonomous vacuum of clause 28, wherein the anti-choking protrusion is a substantially thin and perpendicular to the vacuum stack inlet.

Clause 30. The autonomous vacuum of clause 16, further comprising: an optical sensor system positioned within the bin cavity and configured to measure a liquid level of liquid waste collected by the waste bag.

Clause 31. The autonomous vacuum of clause 30, wherein the optical sensor system comprises: one or more light emitters positioned adjacent to the waste bag and proximate to a fill line of the waste bag and configured to emit light towards the waste bag; one or more light receivers positioned adjacent to the waste bag and proximate to the fill line of the waste bag and configured to measure light incident on the light receivers; and a controller configured to: instruct light emission by the light emitters, receive light incident on the light receivers, and detect the liquid level of liquid waste based on the light incident on the light receivers.

Clause 32. The autonomous vacuum of clause 31, wherein the light emitters emit light in the infrared spectrum, wherein the light receivers detect light in the infrared spectrum, and, optionally, wherein a non-permeable portion of the waste bag is composed of an infrared-transparent material.

Clause 33. The autonomous vacuum of clause 31, wherein the optical sensor system comprises three emitter-receiver pairs positioned at three different heights.

Clause 34. The autonomous vacuum of clause 33, wherein the controller is further configured to: quantify the liquid level based on the measured light from the three emitter-receiver pairs.

Clause 35. The autonomous vacuum of clause 31, wherein the controller of the optical sensor system is further configured to: detect the waste bag is wet-full based on the detected liquid level; and responsive to detecting the waste bag is wet-full, terminate operation of the autonomous vacuum, and, optionally, prompt a user of the autonomous vacuum to replace the waste bag.

Clause 36. The autonomous vacuum of clause 16, further comprising: a capacitive sensing system positioned on an inner surface of the bin cavity to be proximate to a waste bag installed in the bin cavity, the capacitive sensing system comprising: an antenna comprising one or more capacitive sensing electrodes configured to measure a surface capacitance in the bin cavity, and a controller coupled to the antenna and configured to determine whether the waste bag is wet-full based on a change in the surface capacitance measured by the antenna.

Clause 37. The autonomous vacuum of clause 16, further comprising: a pressure sensor system positioned along an airflow pathway of the autonomous vacuum and configured to measure a dry level of dry waste collected by the waste bag.

Clause 38. The autonomous vacuum of clause 37, wherein the pressure sensor system comprises: a pressure a plurality of pressure ports positioned along an airflow pathway; a plurality of pressure sensors connected to the pressure ports and configured to measure pressures at the plurality of pressure ports; and a controller configured to: receive pressure readings from the plurality of pressure sensors, and detect the dry level of the dry waste collected by the waste bag based on the pressure readings.

Clause 39. The autonomous vacuum of clause 38, wherein the pressure sensor system includes: a first pressure port positioned in a channel between the cleaning head and the bin cavity, and a first corresponding pressure sensor connected to the first pressure port; and a second pressure port positioned in the bin cavity, and a second corresponding pressure sensor connected to the second pressure port, wherein the controller is further configured to: detect the dry level of the dry waste based on a pressure differential of a first pressure reading at the first pressure port and a second pressure reading at the second pressure port.

Clause 40. The autonomous vacuum of clause 38, further comprising: a communication multiplexer configured to relay the pressure readings from the plurality of pressure sensors to the controller.

Clause 41. The autonomous vacuum of clause 38, wherein the controller of the pressure sensor system is further configured to: detect the waste bag is dry-full based on the detected dry level; and responsive to detecting the waste bag is dry-full, terminate operation of the autonomous vacuum, and, optionally, prompt a user of the autonomous vacuum to replace the waste bag.

Clause 42. The autonomous vacuum of clause 16, further comprising: a humidity sensor positioned within the bin cavity and configured to measure a humidity level inside the bin cavity.

Clause 43. The autonomous vacuum of clause 42, further comprising: a controller configured to: receive the humidity level measured by the humidity sensor; and perform a ventilation operation based on the humidity level.

Clause 44. The autonomous vacuum of clause 43, wherein performing the ventilation operation comprises one or both of: opening a lid of the autonomous vacuum to vent humid air out of the bin cavity; and operating the vacuum motor at a low speed to vent humid air through an exhaust.

Clause 45. The autonomous vacuum of clause 16, further comprising: a solvent tank storing solvent with a chemical agent for sterilization of liquid waste ingested by autonomous vacuum; and a solvent dispersion system positioned in the cleaning head of the autonomous vacuum and configured to dispense the solvent from the solvent tank into a cleaning environment of the autonomous vacuum.

Clause 46. A computer-implemented method comprising: performing, with an autonomous vacuum, a cleaning operation by engaging a vacuum motor of the autonomous vacuum, wherein the cleaning operation includes mopping; instructing light emission by one or more light emitters of an optical sensor system positioned adjacent to a waste bag of the autonomous vacuum and proximate to a fill line of the waste bag; receiving light incident on one or more light receivers of the optical sensor system positioned adjacent to the waste bag and proximate to the fill line of the waste bag; determining a liquid level of liquid waste in the waste bag based on the light incident on the one or more light receivers; determining the waste bag to be full of liquid waste by determining the liquid level of the liquid waste has reached the fill line; and responsive to determining that the waste bag is full of liquid waste, terminating the cleaning operation and prompting a user of the autonomous vacuum to replace the waste bag.

Clause 47. A computer-implemented method comprising: performing, with an autonomous vacuum, a cleaning operation by engaging a vacuum motor of the autonomous vacuum at a default speed; during the cleaning operation, receiving pressure readings from a plurality of pressure sensors coupled to a plurality of pressure ports positioned along an airflow pathway of the autonomous vacuum, wherein the pressure readings include: a first pressure reading at a first pressure port positioned in a channel between a cleaning head of the autonomous vacuum and a bin cavity of the autonomous vacuum, which holds a waste bag, a second pressure reading at a second pressure port positioned in the bin cavity of the autonomous vacuum; determining whether a pressure differential between the first pressure reading and the second pressure reading is above a low threshold; responsive to determining that the pressure differential between the first pressure reading and the second pressure reading is above the low threshold, performing a purge of the airflow pathway by engaging the vacuum motor to a high speed that is greater than the default speed; receiving subsequent pressure readings including a third pressure reading at the first pressure port and a fourth pressure reading at the second pressure port; determining the waste bag is full of dry waste by determining a pressure differential between the third pressure reading and the fourth pressure reading is above a medium threshold that is higher than the low threshold; and responsive to determining that the waste bag is full of dry waste, terminating the cleaning operation and prompting a user of the autonomous vacuum to replace the waste bag.

### ADDITIONAL CONSIDERATIONS

The foregoing description of the embodiments disclosed have been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments is intended to be illustrative, but not limiting, of the scope of the embodiments disclosed, which is set forth in the following claims.

What is claimed is:

1. A semi-waterproof waste bag for an autonomous vacuum, the waste bag comprising:
a non-permeable portion composed of a waterproof material and structured to hold liquid waste from the autonomous vacuum; and
a permeable portion composed of an air-permeable material to filter out dry contaminants from air, the permeable portion affixed atop the non-permeable portion, and the permeable portion having a first opening for waste to enter the semi-waterproof waste bag.

2. The semi-waterproof waste bag of claim 1, wherein the first opening is for dry waste from a main channel of the autonomous vacuum.

3. The semi-waterproof waste bag of claim 2, wherein the permeable portion includes one or more secondary openings for liquid waste to enter the semi-waterproof waste bag from one or more secondary channels of the autonomous vacuum.

4. The semi-waterproof waste bag of claim 3, wherein the semi-waterproof waste bag further comprises:
one or more guide vanes disposed within the semi-waterproof waste bag to guide liquid waste entering the semi-waterproof waste bag away from the permeable portion and towards the non-permeable portion.

5. The semi-waterproof waste bag of claim 1, further comprising:
a bag tab formed of a rigid material, the bag tab affixed to the permeable portion, the bag tab configured to slot into a tab receiver of the autonomous vacuum to secure the semi-waterproof waste bag in a bin cavity of the autonomous vacuum.

6. The semi-waterproof waste bag of claim 5, wherein the bag tab includes an opening that aligns with the first opening of the permeable portion.

7. The semi-waterproof waste bag of claim 6, wherein the bag tab further comprises a gasket of annular shape formed of a compliant material that aligns to the opening of the bag tab, wherein the gasket forms an air-tight seal between the semi-waterproof waste bag and the bin cavity of the autonomous vacuum when the semi-waterproof waste bag is secured in the bin cavity of the autonomous vacuum.

8. The semi-waterproof waste bag of claim 7, wherein the bag tab is substantially planar, wherein the bag tab includes a bag tab bump on a back face of the bag tab that faces the permeable portion of the semi-waterproof waste bag, and wherein a bump on the tab receiver applies a lateral force on the bag tab bump when the semi-waterproof waste bag is secured in the bin cavity of the autonomous vacuum.

9. The semi-waterproof waste bag of claim 1, further comprising:
a film composed of a waterproof material, disposed along a wall of an interior of the semi-waterproof waste bag, affixed to an underside of a top face of the permeable portion, and substantially coextensive to the top face of the permeable portion.

10. The semi-waterproof waste bag of claim 1, wherein two corners of the permeable portion of the waste bag are joined together.

11. The semi-waterproof waste bag of claim 1, further comprising:
a rigid cage coupled to an inner surface of the non-permeable portion, the rigid cage including one or more openings to an interior of the waste bag and structured to hold a chemical agent for sterilization of waste in the waste bag, or
a sachet tethered to an inner surface of the permeable portion, the sachet formed of a permeable material and storing a chemical agent for sterilization of air in the waste bag.

12. An autonomous vacuum comprising:
a vacuum stack including a vacuum motor configured to generate a vacuum force;
a bin cavity formed to fit a waste bag, wherein the bin cavity includes:
a cavity opening that connects to a cleaning head of the autonomous vacuum where waste is ingested into the autonomous vacuum,
a tab receiver including two receiving elements positioned at a width of a bag tab of the waste bag, wherein the tab receiver is positioned to be centered around the cavity opening, and
a vacuum stack inlet that connects the bin cavity to the vacuum stack.

13. The autonomous vacuum of claim 12, further comprising:
a bag-bin seal formed of a compliant material and positioned around the cavity opening of the bin cavity.

14. The autonomous vacuum of claim 13, wherein the bag-bin seal includes:
a planar portion of annular shape, wherein an inside diameter of the annular shape matches to the diameter of the cavity opening;
an inner ring that is substantially perpendicular to the planar portion; and
an outer ring of arcuate shape that bends towards a center axis of the bag-bin seal.

15. The autonomous vacuum of claim 14, wherein the inner ring extends into the waste bag when the bag tab of the waste bag is secured into the tab receiver.

16. The autonomous vacuum of claim 14, wherein the outer ring forms an air-tight seal when the bag tab of the waste bag is secured into the tab receiver.

17. The autonomous vacuum of claim 12, further comprising:
a lid positioned to cover the bin cavity to form an airtight seal during operation of the vacuum motor, the lid comprising:
a top portion;
a bottom portion affixed to the top portion and including an array of holes that connect to the bin cavity when the lid covers the bin cavity; and
an elastic membrane affixed to the top portion, positioned in between the top portion and the bottom portion, aligned to the array of holes, and configured to deform to block the array of holes during operation of the vacuum motor.

18. The autonomous vacuum of claim 12, further comprising:
an optical sensor system positioned within the bin cavity and configured to measure a liquid level of liquid waste collected by the waste bag, the optical sensor system comprising:
one or more light emitters positioned adjacent to the waste bag and proximate to a fill line of the waste bag and configured to emit light towards the waste bag;
one or more light receivers positioned adjacent to the waste bag and proximate to the fill line of the waste bag and configured to measure light incident on the light receivers; and
a controller configured to:
instruct light emission by the light emitters,
receive light incident on the light receivers, and
detect the liquid level of liquid waste based on the light incident on the light receivers.

19. The autonomous vacuum of claim 12, further comprising:
a capacitive sensing system positioned on an inner surface of the bin cavity to be proximate to a waste bag installed in the bin cavity, the capacitive sensing system comprising:
an antenna comprising one or more capacitive sensing electrodes configured to measure a surface capacitance in the bin cavity, and
a controller coupled to the antenna and configured to determine whether the waste bag is wet-full based on a change in the surface capacitance measured by the antenna.

20. The autonomous vacuum of claim 12, further comprising:
a pressure sensor system positioned along an airflow pathway of the autonomous vacuum and configured to measure a dry level of dry waste collected by the waste bag, the pressure sensor system comprising:
a pressure a plurality of pressure ports positioned along an airflow pathway;
a plurality of pressure sensors connected to the pressure ports and configured to measure pressures at the plurality of pressure ports; and
a controller configured to:
receive pressure readings from the plurality of pressure sensors, and
detect the dry level of the dry waste collected by the waste bag based on the pressure readings.

21. The autonomous vacuum of claim 12, further comprising:
a humidity sensor positioned within the bin cavity and configured to measure a humidity level inside the bin cavity, and
a controller configured to:
receive the humidity level measured by the humidity sensor; and
perform a ventilation operation based on the humidity level.

22. The autonomous vacuum of claim 12, further comprising:
a solvent tank storing solvent with a chemical agent for sterilization of liquid waste ingested by autonomous vacuum; and
a solvent dispersion system positioned in the cleaning head of the autonomous vacuum and configured to dispense the solvent from the solvent tank into a cleaning environment of the autonomous vacuum.
